SEQUENCE LISTING (457 sequences)

SEQ ID NO: 1 - protein, 20 aa, Artificial Sequence
  markush structure
  MISC_FEATURE (2)..(2): X is H, H-3Me or PyA-4
  MISC_FEATURE (6)..(6): X is T, S, D or E
  MISC_FEATURE (7)..(7): X is F, F-2-Br, F-2-Cl or F-3-F
  MISC_FEATURE (8)..(8): X is W, Nal, or absent
  MISC_FEATURE (10)..(10): X is D, or N-Me-D
  MISC_FEATURE (11)..(11): X is G, A, or P
  MISC_FEATURE (12)..(12): X is E or D
  MISC_FEATURE (14)..(14): X is K, or N-Me-K
  MISC_FEATURE (19)..(19): X is T, or N-Me-T
TXLETXXXGXXXPXTIRVXQ

SEQ ID NO: 2 - protein, 23 aa, Artificial Sequence
  markush structure
  MISC_FEATURE (2)..(2): X is H, H-3Me, or PyA-4
  MISC_FEATURE (6)..(6): X is T, S, D, or E
  MISC_FEATURE (7)..(7): X is F, F-2-Br, F-2-Cl, or F-3-F
  MISC_FEATURE (8)..(8): X is W, Nal, or absent
  MISC_FEATURE (10)..(10): X is D, or N-Me-D
  MISC_FEATURE (11)..(11): X is G, A, or P
  MISC_FEATURE (12)..(12): X is E, or D
  MISC_FEATURE (14)..(14): X is K, or N-Me-K
  MISC_FEATURE (19)..(19): X is T, or N-Me-T
  MISC_FEATURE (21)..(21): is D-proline
  MISC_FEATURE (21)..(21)
  MISC_FEATURE (23)..(23): X is C, Q, or E
TXLETXXXGXXXPXTIRVXQPGX

SEQ ID NO: 3 - protein, 19 aa, Artificial Sequence
  markush structure
  MISC_FEATURE (2)..(2): X is H, H-3Me, or PyA-4
  MISC_FEATURE (6)..(6): X is T, S, D, or E
  MISC_FEATURE (7)..(7): X is F, F-2-Br, F-2-Cl, or F-3-F
  MISC_FEATURE (9)..(9): X is D, or N-Me-D
  MISC_FEATURE (10)..(10): X is G, A, or P
  MISC_FEATURE (11)..(11): X is E, or D
  MISC_FEATURE (13)..(13): X is K, or N-Me-K
  MISC_FEATURE (18)..(18): X is T, or N-Me-T
TXLETXXGXXXPXTIRVXQ

SEQ ID NO: 4 - protein, 22 aa, Artificial Sequence
  markush structure
  MISC_FEATURE (2)..(2): X is H, H-3Me, or PyA-4
  MISC_FEATURE (6)..(6): X is T, S, D, or E
  MISC_FEATURE (7)..(7): X is F, F-2-Br, F-2-Cl, or F-3-F
  MISC_FEATURE (9)..(9): X is D, or N-Me-D
  MISC_FEATURE (10)..(10): X is G, A, or P
  MISC_FEATURE (11)..(11): X is E, or D
  MISC_FEATURE (13)..(13): X is K, or N-Me-K
  MISC_FEATURE (18)..(18): X is T, or N-Me-T
  MISC_FEATURE (20)..(20): is D-proline
  MISC_FEATURE (22)..(22): X is C, Q, or E
TXLETXXGXXXPXTIRVXQPGX

SEQ ID NO: 5 - protein, 23 aa, Artificial Sequence
  markush structure
  MISC_FEATURE (2)..(2): X is H, H-3Me, or PyA-4
  MISC_FEATURE (8)..(8): X is W, Nal, or absent
  MISC_FEATURE (10)..(10): X is D, or N-Me-D
  MISC_FEATURE (11)..(11): X is G, A, or P
  MISC_FEATURE (12)..(12): X is E, or D
  MISC_FEATURE (14)..(14): X is K, or N-Me-K
  MISC_FEATURE (19)..(19): X is T, or N-Me-T
  MISC_FEATURE (21)..(21): is D-proline
  MISC_FEATURE (23)..(23): X is C, Q, or E
TXLETTFXGXXXPXTIRVXQPGX

SEQ ID NO: 6 - protein, 22 aa, Artificial Sequence
  markush structure
  MISC_FEATURE (2)..(2): X is H, H-3Me, or PyA-4
  MISC_FEATURE (9)..(9): X is D, or N-Me-D
  MISC_FEATURE (10)..(10): X is G, A, or P
  MISC_FEATURE (11)..(11): X is E, or D
  MISC_FEATURE (13)..(13): X is K, or N-Me-K
  MISC_FEATURE (18)..(18): X is T, or N-Me-T
  MISC_FEATURE (20)..(20): is D-proline
  MISC_FEATURE (22)..(22): X is C, Q, or E
TXLETTFGXXXPXTIRVXQPGX

SEQ ID NO: 7 - protein, 20 aa, Artificial Sequence
  markush structure
  MISC_FEATURE (2)..(2): X is H or PyA-4
  MISC_FEATURE (8)..(8): X is W or Nal
  MISC_FEATURE (12)..(12): X is E or D
  MISC_FEATURE (14)..(14): X is K or N-Me-K
  MISC_FEATURE (19)..(19): X is T or N-Me-T
TXLETTFXGDGXPXTIRVXQ

SEQ ID NO: 8 - protein, 12 aa, Artificial Sequence
  synthetic peptide
THLETTFWGDGE

SEQ ID NO: 9 - protein, 12 aa, Artificial Sequence
  synthetic peptide
THLETTFWGDGD

SEQ ID NO: 10 - protein, 12 aa, Artificial Sequence
  synthetic peptide
  MISC_FEATURE (8)..(8): X is Nal
THLETTFXGDGE

SEQ ID NO: 11 - protein, 12 aa, Artificial Sequence
  synthetic peptide
  MISC_FEATURE (2)..(2): X is PyA-4
TXLETTFWGDGE

SEQ ID NO: 12 - protein, 23 aa, Artificial Sequence
  cyclized peptide
  MISC_FEATURE (21)..(21): is D-proline
THLETTFTGDGTPKTIRVTQPGQ

SEQ ID NO: 13 - protein, 23 aa, Artificial Sequence
  markush structure
  MISC_FEATURE (2)..(2): X is H, or H-3Me
  MISC_FEATURE (8)..(8): X is W, or absent
  MISC_FEATURE (21)..(21): is D-proline
  MISC_FEATURE (23)..(23): X is C, Q, or E
TXLETTFXGDGEPKTIRVTQPGX

SEQ ID NO: 14 - protein, 23 aa, Artificial Sequence
  cyclized peptide
  MISC_FEATURE (21)..(21): is D-AA
THLETTFWGDGEPKTIRVTQPGQ

SEQ ID NO: 15 - protein, 22 aa, Artificial Sequence
  cyclized peptide
  MISC_FEATURE (20)..(20): is D-AA
THLETTFGDGEPKTIRVTQPGQ

SEQ ID NO: 16 - protein, 23 aa, Artificial Sequence
  cyclized peptide
  MISC_FEATURE (2)..(2): X is H-3Me
  MISC_FEATURE (21)..(21): is D-AA
TXLETTFWGDGEPKTIRVTQPGQ

SEQ ID NO: 17 - protein, 22 aa, Artificial Sequence
  cyclized peptide
  MISC_FEATURE (2)..(2): X is H-3Me
  MISC_FEATURE (20)..(20): is D-AA
TXLETTFGDGEPKTIRVTQPGQ

SEQ ID NO: 18 - protein, 23 aa, Artificial Sequence
  cyclized peptide
  MISC_FEATURE (21)..(21): is D-AA
THLETTFWGDGTPKTIRVTQPGQ

SEQ ID NO: 19 - protein, 23 aa, Artificial Sequence
  cyclized peptide
  MISC_FEATURE (21)..(21): is D-AA
THLETTFDGDGTPKTIRVTQPGQ

SEQ ID NO: 20 - protein, 23 aa, Artificial Sequence
  cyclized peptide
  MISC_FEATURE (21)..(21): is D-AA
THLETTFHGDGTPKTIRVTQPGQ

SEQ ID NO: 21 - protein, 23 aa, Artificial Sequence
  cyclized peptide
  MISC_FEATURE (21)..(21): is D-AA
THLETTVTGDGTPKTIRVTQPGQ

SEQ ID NO: 22 - protein, 23 aa, Artificial Sequence
  cyclized peptide
  MISC_FEATURE (21)..(21): is D-AA
THKETTFTGDGTPKTIRVTQPGQ

SEQ ID NO: 23 - protein, 23 aa, Artificial Sequence
  cyclized peptide
  MISC_FEATURE (21)..(21): is D-AA
TELETTFTGDGTPKTIRVTQPGQ

SEQ ID NO: 24 - protein, 23 aa, Artificial Sequence
  cyclized peptide
  MISC_FEATURE (21)..(21): is D-AA
YHLETTFTGDGTPKTIRVTQPGQ

SEQ ID NO: 25 - protein, 23 aa, Artificial Sequence
  cyclized peptide
  MISC_FEATURE (21)..(21): is D-AA
GHLETTFTGDGTPKTIRVTQPGQ

SEQ ID NO: 26 - protein, 23 aa, Artificial Sequence
  cyclized peptide
  MISC_FEATURE (21)..(21): is D-AA
TYLETTFTGDGTPKTIRVTQPGQ

SEQ ID NO: 27 - protein, 23 aa, Artificial Sequence
  cyclized peptide
  MISC_FEATURE (21)..(21): is D-AA
TFLETTFTGDGTPKTIRVTQPGQ

SEQ ID NO: 28 - protein, 23 aa, Artificial Sequence
  cyclized peptide
  MISC_FEATURE (21)..(21): is D-AA
THLETTFTGKGTPKTIRVTQPGQ

SEQ ID NO: 29 - protein, 23 aa, Artificial Sequence
  cyclized peptide
  MISC_FEATURE (21)..(21): is D-AA
THLETTFTGPGTPKTIRVTQPGQ

SEQ ID NO: 30 - protein, 23 aa, Artificial Sequence
  cyclized peptide
  MISC_FEATURE (21)..(21): is D-AA
THLETTFTGDPTPKTIRVTQPGQ

SEQ ID NO: 31 - protein, 23 aa, Artificial Sequence
  cyclized peptide
  MISC_FEATURE (21)..(21): is D-AA
THLETTFTGDGPPKTIRVTQPGQ

SEQ ID NO: 32 - protein, 23 aa, Artificial Sequence
  cyclized peptide
  MISC_FEATURE (21)..(21): is D-AA
THLETTFTGDGDPKTIRVTQPGQ

SEQ ID NO: 33 - protein, 23 aa, Artificial Sequence
  cyclized peptide
  MISC_FEATURE (21)..(21): is D-AA
THLETTFTGDGEPKTIRVTQPGQ

SEQ ID NO: 34 - protein, 23 aa, Artificial Sequence
  cyclized peptide
  MISC_FEATURE (21)..(21): is D-AA
THLETTFTGRGTPKTIRVTQPGQ

SEQ ID NO: 35 - protein, 23 aa, Artificial Sequence
  cyclized peptide
  MISC_FEATURE (21)..(21): is D-AA
THLETTFTGDRTPKTIRVTQPGQ

SEQ ID NO: 36 - protein, 23 aa, Artificial Sequence
  cyclized peptide
  MISC_FEATURE (21)..(21): is D-AA
THLETTFTGDGWPKTIRVTQPGQ

SEQ ID NO: 37 - protein, 23 aa, Artificial Sequence
  cyclized peptide
  MISC_FEATURE (21)..(21): is D-AA
THLETTFMGDGTPKTIRVTQPGQ

SEQ ID NO: 38 - protein, 23 aa, Artificial Sequence
  cyclized peptide
  MISC_FEATURE (21)..(21): is D-AA
THLETTFTGDGCPKTIRVTQPGQ

SEQ ID NO: 39 - protein, 23 aa, Artificial Sequence
  cyclized peptide
  MISC_FEATURE (21)..(21): is D-AA
THLETTFTGDGTPKNIRVTQPGQ

SEQ ID NO: 40 - protein, 23 aa, Artificial Sequence
  cyclized peptide
  MISC_FEATURE (21)..(21): is D-AA
THLETTFTGDGNPKTIRVTQPGQ

SEQ ID NO: 41 - protein, 23 aa, Artificial Sequence
  cyclized peptide
  misc (21)..(21): is D-AA
THLETTFTGDGTPKTRRVTQPGQ

SEQ ID NO: 42 - protein, 23 aa, Artificial Sequence
  cyclized peptide
  MISC_FEATURE (21)..(21): is D-AA
THLETTFTGDGTPKTYRVTQPGQ

SEQ ID NO: 43 - protein, 23 aa, Artificial Sequence
  cyclized peptide
  MISC_FEATURE (21)..(21): is D-AA
THLETTFTGDGTPKTIRRTQPGQ

SEQ ID NO: 44 - protein, 23 aa, Artificial Sequence
  cyclized peptide
  MISC_FEATURE (21)..(21): is D-AA
THLETTFTGDGTPKTIRVRQPGQ

SEQ ID NO: 45 - protein, 23 aa, Artificial Sequence
  cyclized peptide
  MISC_FEATURE (21)..(21): is D-AA
THLETTFTGDGTPKTIRVRRPGQ

SEQ ID NO: 46 - protein, 23 aa, Artificial Sequence
  cyclized peptide
  MISC_FEATURE (21)..(21): is D-AA
THLETTFTGDGTPKTIRFTQPGQ

SEQ ID NO: 47 - protein, 23 aa, Artificial Sequence
  cyclized peptide
  MISC_FEATURE (21)..(21): is D-AA
THLETTFTGDGTPKTIRYTQPGQ

SEQ ID NO: 48 - protein, 23 aa, Artificial Sequence
  cyclized peptide
  MISC_FEATURE (21)..(21): is D-AA
THLETTFTGDGTPKTIRVFQPGQ

SEQ ID NO: 49 - protein, 23 aa, Artificial Sequence
  cyclized peptide
  MISC_FEATURE (21)..(21): is D-AA
THLETTFTGDGTPKTIRVYQPGQ

SEQ ID NO: 50 - protein, 23 aa, Artificial Sequence
  cyclized peptide
  MISC_FEATURE (21)..(21): is D-AA
THLETTFTGDGTPKSIRVTQPGQ

SEQ ID NO: 51 - protein, 23 aa, Artificial Sequence
  cyclized peptide
  MISC_FEATURE (21)..(21): is D-AA
THLETTFTGGGTPKTIRVTQPGQ

SEQ ID NO: 52 - protein, 23 aa, Artificial Sequence
  cyclized peptide
  MISC_FEATURE (21)..(21): is D-AA
THLESTFTGDGTPKTIRVTQPGQ

SEQ ID NO: 53 - protein, 23 aa, Artificial Sequence
  cyclized peptide
  MISC_FEATURE (21)..(21): is D-AA
THLETEFTGDGTPKTIRVTQPGQ

SEQ ID NO: 54 - protein, 23 aa, Artificial Sequence
  cyclized peptide
  MISC_FEATURE (21)..(21): is D-AA
THLETDFTGDGTPKTIRVTQPGQ

SEQ ID NO: 55 - protein, 23 aa, Artificial Sequence
  cyclized peptide
  MISC_FEATURE (21)..(21): is D-AA
AHLETTFTGDGTPKTIRVTQPGQ

SEQ ID NO: 56 - protein, 23 aa, Artificial Sequence
  cyclized peptide
  MISC_FEATURE (21)..(21): is D-AA
TALETTFTGDGTPKTIRVTQPGQ

SEQ ID NO: 57 - protein, 23 aa, Artificial Sequence
  cyclized peptide
  MISC_FEATURE (21)..(21): is D-AA
THAETTFTGDGTPKTIRVTQPGQ

SEQ ID NO: 58 - protein, 23 aa, Artificial Sequence
  cyclized peptide
  MISC_FEATURE (21)..(21): is D-AA
THLATTFTGDGTPKTIRVTQPGQ

SEQ ID NO: 59 - protein, 23 aa, Artificial Sequence
  cyclized peptide
  MISC_FEATURE (21)..(21): is D-AA
THLEATFTGDGTPKTIRVTQPGQ

SEQ ID NO: 60 - protein, 23 aa, Artificial Sequence
  cyclized peptide
  MISC_FEATURE (21)..(21): is D-AA
THLETAFTGDGTPKTIRVTQPGQ

SEQ ID NO: 61 - protein, 23 aa, Artificial Sequence
  cyclized peptide
  MISC_FEATURE (21)..(21): is D-AA
THLETTATGDGTPKTIRVTQPGQ

SEQ ID NO: 62 - protein, 23 aa, Artificial Sequence
  cyclized peptide
  MISC_FEATURE (21)..(21): is D-AA
THLETTFAGDGTPKTIRVTQPGQ

SEQ ID NO: 63 - protein, 23 aa, Artificial Sequence
  cyclized peptide
  MISC_FEATURE (21)..(21): is D-AA
THLETTFTADGTPKTIRVTQPGQ

SEQ ID NO: 64 - protein, 23 aa, Artificial Sequence
  cyclized peptide
  MISC_FEATURE (21)..(21): is D-AA
THLETTFTGAGTPKTIRVTQPGQ

SEQ ID NO: 65 - protein, 23 aa, Artificial Sequence
  cyclized peptide
  MISC_FEATURE (21)..(21): is D-AA
THLETTFTGDATPKTIRVTQPGQ

SEQ ID NO: 66 - protein, 23 aa, Artificial Sequence
  cyclized peptide
  MISC_FEATURE (21)..(21): is D-AA
THLETTFTGDGAPKTIRVTQPGQ

SEQ ID NO: 67 - protein, 23 aa, Artificial Sequence
  cyclized peptide
  MISC_FEATURE (21)..(21): is D-AA
THLETTFTGDGTAKTIRVTQPGQ

SEQ ID NO: 68 - protein, 23 aa, Artificial Sequence
  cyclized peptide
  MISC_FEATURE (21)..(21): is D-AA
THLETTFTGDGTPATIRVTQPGQ

SEQ ID NO: 69 - protein, 23 aa, Artificial Sequence
  cyclized peptide
  MISC_FEATURE (21)..(21): is D-AA
THLETTFTGDGTPKAIRVTQPGQ

SEQ ID NO: 70 - protein, 23 aa, Artificial Sequence
  cyclized peptide
  MISC_FEATURE (21)..(21): is D-AA
THLETTFTGDGTPKTARVTQPGQ

SEQ ID NO: 71 - protein, 23 aa, Artificial Sequence
  cyclized peptide
  MISC_FEATURE (21)..(21): is D-AA
THLETTFTGDGTPKTIAVTQPGQ

SEQ ID NO: 72 - protein, 23 aa, Artificial Sequence
  cyclized peptide
  MISC_FEATURE (21)..(21): is D-AA
THLETTFTGDGTPKTIRATQPGQ

SEQ ID NO: 73 - protein, 23 aa, Artificial Sequence
  cyclized peptide
  MISC_FEATURE (21)..(21): is D-AA
THLETTFTGDGTPKTIRVAQPGQ

SEQ ID NO: 74 - protein, 23 aa, Artificial Sequence
  cyclized peptide
  MISC_FEATURE (21)..(21): is D-AA
THLETTFTGDGTPKTIRVTAPGQ

SEQ ID NO: 75 - protein, 22 aa, Artificial Sequence
  cyclized peptide
  MISC_FEATURE (20)..(20): is D-AA
THLETTFGDGTPKTIRVTQPGQ

SEQ ID NO: 76 - protein, 19 aa, Artificial Sequence
  cyclized peptide
  MISC_FEATURE (17)..(17): is D-AA
THLETTFTPKTIRVTQPGQ

SEQ ID NO: 77 - protein, 22 aa, Artificial Sequence
  cyclized peptide
  MISC_FEATURE (20)..(20): is D-AA
THLETTFTGDGPKTIRVTQPGQ

SEQ ID NO: 78 - protein, 22 aa, Artificial Sequence
  cyclized peptide
  MISC_FEATURE (20)..(20): is D-AA
THLETTFTGGTPKTIRVTQPGQ

SEQ ID NO: 79 - protein, 22 aa, Artificial Sequence
  cyclized peptide
  MISC_FEATURE (20)..(20): is D-AA
THLETTFTGDTPKTIRVTQPGQ

SEQ ID NO: 80 - protein, 23 aa, Artificial Sequence
  cyclized peptide
  MISC_FEATURE (5)..(5): X is AlloT
  MISC_FEATURE (21)..(21): is D-AA
THLEXTFTGDGTPKTIRVTQPGQ

SEQ ID NO: 81 - protein, 23 aa, Artificial Sequence
  cyclized peptide
  MISC_FEATURE (6)..(6): X is AlloT
  MISC_FEATURE (21)..(21): is D-AA
THLETXFTGDGTPKTIRVTQPGQ

SEQ ID NO: 82 - protein, 23 aa, Artificial Sequence
  cyclized peptide
  MISC_FEATURE (1)..(1): X is AlloT
  MISC_FEATURE (21)..(21): is D-AA
XHLETTFTGDGTPKTIRVTQPGQ

SEQ ID NO: 83 - protein, 23 aa, Artificial Sequence
  cyclized peptide
  MISC_FEATURE (15)..(15): X is AlloT
  MISC_FEATURE (21)..(21): is D-AA
THLETTFTGDGTPKXIRVTQPGQ

SEQ ID NO: 84 - protein, 23 aa, Artificial Sequence
  cyclized peptide
  MISC_FEATURE (1)..(1): X is N-Me-T
  MISC_FEATURE (21)..(21): is D-AA
XHLETTFTGDGTPKTIRVTQPGQ

SEQ ID NO: 85 - protein, 23 aa, Artificial Sequence
  cyclized peptide
  MISC_FEATURE (2)..(2): X is N-Me-H
  MISC_FEATURE (21)..(21): is D-AA
TXLETTFTGDGTPKTIRVTQPGQ

SEQ ID NO: 86 - protein, 23 aa, Artificial Sequence
  cyclized peptide
  MISC_FEATURE (3)..(3): X is N-Me-L
  MISC_FEATURE (21)..(21): is D-AA
THXETTFTGDGTPKTIRVTQPGQ

SEQ ID NO: 87 - protein, 23 aa, Artificial Sequence
  cyclized peptide
  MISC_FEATURE (4)..(4): X is N-Me-E
  MISC_FEATURE (21)..(21): is D-AA
THLXTTFTGDGTPKTIRVTQPGQ

SEQ ID NO: 88 - protein, 23 aa, Artificial Sequence
  cyclized peptide
  MISC_FEATURE (5)..(5): X is N-Me-T
  MISC_FEATURE (21)..(21): is D-AA
THLEXTFTGDGTPKTIRVTQPGQ

SEQ ID NO: 89 - protein, 23 aa, Artificial Sequence
  cyclized peptide
  MISC_FEATURE (6)..(6): X is N-Me-T
  MISC_FEATURE (21)..(21): is D-AA
THLETXFTGDGTPKTIRVTQPGQ

SEQ ID NO: 90 - protein, 23 aa, Artificial Sequence
  cyclized peptide
  MISC_FEATURE (7)..(7): X is N-Me-F
  MISC_FEATURE (21)..(21): is D-AA
THLETTXTGDGTPKTIRVTQPGQ

SEQ ID NO: 91 - protein, 23 aa, Artificial Sequence
  cyclized peptide
  MISC_FEATURE (8)..(8): X is N-Me-T
  MISC_FEATURE (21)..(21): is D-AA
THLETTFXGDGTPKTIRVTQPGQ

SEQ ID NO: 92 - protein, 23 aa, Artificial Sequence
  cyclized peptide
  MISC_FEATURE (9)..(9): X is N-Me-G
  MISC_FEATURE (21)..(21): is D-AA
THLETTFTXDGTPKTIRVTQPGQ

SEQ ID NO: 93 - protein, 23 aa, Artificial Sequence
  cyclized peptide
  MISC_FEATURE (10)..(10): X is N-Me-D
  MISC_FEATURE (21)..(21): is D-AA
THLETTFTGXGTPKTIRVTQPGQ

SEQ ID NO: 94 - protein, 23 aa, Artificial Sequence
  cyclized peptide
  MISC_FEATURE (11)..(11): X is N-Me-G
  MISC_FEATURE (21)..(21): is D-AA
THLETTFTGDXTPKTIRVTQPGQ

SEQ ID NO: 95 - protein, 23 aa, Artificial Sequence
  cyclized peptide
  MISC_FEATURE (12)..(12): X is N-Me-T
  MISC_FEATURE (21)..(21): is D-AA
THLETTFTGDGXPKTIRVTQPGQ

SEQ ID NO: 96 - protein, 23 aa, Artificial Sequence
  cyclized peptide
  MISC_FEATURE (14)..(14): X is N-Me-K
  MISC_FEATURE (21)..(21): is D-AA
THLETTFTGDGTPXTIRVTQPGQ

SEQ ID NO: 97 - protein, 23 aa, Artificial Sequence
  cyclized peptide
  MISC_FEATURE (15)..(15): X is N-Me-T
  MISC_FEATURE (21)..(21): is D-AA
THLETTFTGDGTPKXIRVTQPGQ

SEQ ID NO: 98 - protein, 23 aa, Artificial Sequence
  cyclized peptide
  MISC_FEATURE (16)..(16): X is N-Me-I
  MISC_FEATURE (21)..(21): is D-AA
THLETTFTGDGTPKTXRVTQPGQ

SEQ ID NO: 99 - protein, 23 aa, Artificial Sequence
  cyclized peptide
  MISC_FEATURE (17)..(17): X is N-Me-R
  MISC_FEATURE (21)..(21): is D-AA
THLETTFTGDGTPKTIXVTQPGQ

SEQ ID NO: 100 - protein, 23 aa, Artificial Sequence
  cyclized peptide
  MISC_FEATURE (18)..(18): X is N-Me-V
  MISC_FEATURE (21)..(21): is D-AA
THLETTFTGDGTPKTIRXTQPGQ

SEQ ID NO: 101 - protein, 23 aa, Artificial Sequence
  cyclized peptide
  MISC_FEATURE (19)..(19): X is N-Me-T
  MISC_FEATURE (21)..(21): is D-AA
THLETTFTGDGTPKTIRVXQPGQ

SEQ ID NO: 102 - protein, 23 aa, Artificial Sequence
  cyclized peptide
  MISC_FEATURE (20)..(20): X is N-Me-Q
  MISC_FEATURE (21)..(21): is D-AA
THLETTFTGDGTPKTIRVTXPGQ

SEQ ID NO: 103 - protein, 23 aa, Artificial Sequence
  cyclized peptide
  MISC_FEATURE (1)..(1): X is pT
  MISC_FEATURE (21)..(21): is D-AA
XHLETTFTGDGTPKTIRVTQPGQ

SEQ ID NO: 104 - protein, 23 aa, Artificial Sequence
  cyclized peptide
  MISC_FEATURE (5)..(5): X is pT
  MISC_FEATURE (21)..(21): is D-AA
THLEXTFTGDGTPKTIRVTQPGQ

SEQ ID NO: 105 - protein, 23 aa, Artificial Sequence
  cyclized peptide
  MISC_FEATURE (6)..(6): X is pT
  MISC_FEATURE (21)..(21): is D-AA
THLETXFTGDGTPKTIRVTQPGQ

SEQ ID NO: 106 - protein, 23 aa, Artificial Sequence
  cyclized peptide
  MISC_FEATURE (8)..(8): X is pT
  MISC_FEATURE (21)..(21): is D-AA
THLETTFXGDGTPKTIRVTQPGQ

SEQ ID NO: 107 - protein, 23 aa, Artificial Sequence
  cyclized peptide
  MISC_FEATURE (12)..(12): X is pT
  MISC_FEATURE (21)..(21): is D-AA
THLETTFTGDGXPKTIRVTQPGQ

SEQ ID NO: 108 - protein, 23 aa, Artificial Sequence
  cyclized peptide
  MISC_FEATURE (15)..(15): X is pT
  MISC_FEATURE (21)..(21): is D-AA
THLETTFTGDGTPKXIRVTQPGQ

SEQ ID NO: 109 - protein, 23 aa, Artificial Sequence
  cyclized peptide
  MISC_FEATURE (19)..(19): X is pT
  MISC_FEATURE (21)..(21): is D-AA
THLETTFTGDGTPKTIRVXQPGQ

SEQ ID NO: 110 - protein, 23 aa, Artificial Sequence
  cyclized peptide
  MISC_FEATURE (2)..(2): X is H-3Me
  MISC_FEATURE (21)..(21): is D-AA
TXLETTFTGDGTPKTIRVTQPGQ

SEQ ID NO: 111 - protein, 23 aa, Artificial Sequence
  cyclized peptide
  MISC_FEATURE (2)..(2): X is H-1Me
  MISC_FEATURE (21)..(21): is D-AA
TXLETTFTGDGTPKTIRVTQPGQ

SEQ ID NO: 112 - protein, 23 aa, Artificial Sequence
  cyclized peptide
  MISC_FEATURE (1)..(1): is D-AA
  MISC_FEATURE (21)..(21): is D-AA
THLETTFTGDGTPKTIRVTQPGQ

SEQ ID NO: 113 - protein, 23 aa, Artificial Sequence
  cyclized peptide
  MISC_FEATURE (2)..(2): is D-AA
  MISC_FEATURE (21)..(21): is D-AA
THLETTFTGDGTPKTIRVTQPGQ

SEQ ID NO: 114 - protein, 22 aa, Artificial Sequence
  cyclized peptide
  MISC_FEATURE (3)..(3): is D-AA
  MISC_FEATURE (21)..(21): is D-AA
THLETTFTGDGTPKTIRVQPGQ

SEQ ID NO: 115 - protein, 23 aa, Artificial Sequence
  cyclized peptide
  MISC_FEATURE (4)..(4): is D-AA
  MISC_FEATURE (21)..(21): is D-AA
THLETTFTGDGTPKTIRVTQPGQ

SEQ ID NO: 116 - protein, 23 aa, Artificial Sequence
  cyclized peptide
  MISC_FEATURE (5)..(5): is D-AA
  MISC_FEATURE (21)..(21): is D-AA
THLETTFTGDGTPKTIRVTQPGQ

SEQ ID NO: 117 - protein, 23 aa, Artificial Sequence
  cyclized peptide
  MISC_FEATURE (6)..(6): is D-AA
  MISC_FEATURE (21)..(21): is D-AA
THLETTFTGDGTPKTIRVTQPGQ

SEQ ID NO: 118 - protein, 23 aa, Artificial Sequence
  cyclized peptide
  MISC_FEATURE (7)..(7): is D-AA
  MISC_FEATURE (21)..(21): is D-AA
THLETTFTGDGTPKTIRVTQPGQ

SEQ ID NO: 119 - protein, 23 aa, Artificial Sequence
  cyclized peptide
  MISC_FEATURE (8)..(8): is D-AA
  MISC_FEATURE (21)..(21): is D-AA
THLETTFTGDGTPKTIRVTQPGQ

SEQ ID NO: 120 - protein, 23 aa, Artificial Sequence
  cyclized peptide
  MISC_FEATURE (10)..(10): is D-AA
  MISC_FEATURE (21)..(21): is D-AA
THLETTFTGDGTPKTIRVTQPGQ

SEQ ID NO: 121 - protein, 23 aa, Artificial Sequence
  cyclized peptide
  MISC_FEATURE (12)..(12): is D-AA
  MISC_FEATURE (21)..(21): is D-AA
THLETTFTGDGTPKTIRVTQPGQ

SEQ ID NO: 122 - protein, 23 aa, Artificial Sequence
  cyclized peptide
  MISC_FEATURE (13)..(13): is D-AA
  MISC_FEATURE (21)..(21): is D-AA
THLETTFTGDGTPKTIRVTQPGQ

SEQ ID NO: 123 - protein, 23 aa, Artificial Sequence
  cyclized peptide
  MISC_FEATURE (14)..(14): is D-AA
  MISC_FEATURE (21)..(21): is D-AA
THLETTFTGDGTPKTIRVTQPGQ

SEQ ID NO: 124 - protein, 23 aa, Artificial Sequence
  cyclized peptide
  MISC_FEATURE (15)..(15): is D-AA
  MISC_FEATURE (21)..(21): is D-AA
THLETTFTGDGTPKTIRVTQPGQ

SEQ ID NO: 125 - protein, 23 aa, Artificial Sequence
  cyclized peptide
  MISC_FEATURE (16)..(16): is D-AA
  MISC_FEATURE (21)..(21): is D-AA
THLETTFTGDGTPKTIRVTQPGQ

SEQ ID NO: 126 - protein, 23 aa, Artificial Sequence
  cyclized peptide
  MISC_FEATURE (17)..(17): is D-AA
  MISC_FEATURE (21)..(21): is D-AA
THLETTFTGDGTPKTIRVTQPGQ

SEQ ID NO: 127 - protein, 23 aa, Artificial Sequence
  cyclized peptide
  MISC_FEATURE (18)..(18): is D-AA
  MISC_FEATURE (21)..(21): is D-AA
THLETTFTGDGTPKTIRVTQPGQ

SEQ ID NO: 128 - protein, 23 aa, Artificial Sequence
  cyclized peptide
  MISC_FEATURE (19)..(19): is D-AA
  MISC_FEATURE (21)..(21): is D-AA
THLETTFTGDGTPKTIRVTQPGQ

SEQ ID NO: 129 - protein, 23 aa, Artificial Sequence
  cyclized peptide
  MISC_FEATURE (20)..(20): is D-AA
  MISC_FEATURE (21)..(21): is D-AA
THLETTFTGDGTPKTIRVTQPGQ

SEQ ID NO: 130 - protein, 23 aa, Artificial Sequence
  cyclized peptide
  MISC_FEATURE (21)..(21): is D-AA
THLETTFWGDGDPKTIRVTQPGQ

SEQ ID NO: 131 - protein, 23 aa, Artificial Sequence
  cyclized peptide
  MISC_FEATURE (21)..(21): is D-AA
THLETTFWGDPTPKTIRVTQPGQ

SEQ ID NO: 132 - protein, 23 aa, Artificial Sequence
  cyclized peptide
  MISC_FEATURE (21)..(21): is D-AA
THLETTFTGDPEPKTIRVTQPGQ

SEQ ID NO: 133 - protein, 23 aa, Artificial Sequence
  cyclized peptide
  MISC_FEATURE (21)..(21): is D-AA
THLETTFWGDPEPKTIRVTQPGQ

SEQ ID NO: 134 - protein, 23 aa, Artificial Sequence
  cyclized peptide
  MISC_FEATURE (21)..(21): is D-AA
THLETTFWGDAEPKTIRVTQPGQ

SEQ ID NO: 135 - protein, 23 aa, Artificial Sequence
  cyclized peptide
  MISC_FEATURE (21)..(21): is D-AA
THLETTFWGDGEPKTIRVTQPGQ

SEQ ID NO: 136 - protein, 22 aa, Artificial Sequence
  cyclized peptide
  MISC_FEATURE (20)..(20): is D-AA
THLETTFGDGEPKTIRVTQPGQ

SEQ ID NO: 137 - protein, 24 aa, Artificial Sequence
  synthetic peptide
  MISC_FEATURE (21)..(21): is D-proline
THLETTFTGDGTPKTIRVTQPGQE

SEQ ID NO: 138 - protein, 23 aa, Artificial Sequence
  cyclized peptide
  MISC_FEATURE (21)..(21): is D-AA
THLETTVTGDGTPKTIRVTQPGQ

SEQ ID NO: 139 - protein, 23 aa, Artificial Sequence
  cyclized peptide
  MISC_FEATURE (21)..(21): is D-AA
THLETTFTGDGEPKTIRVTQPGQ

SEQ ID NO: 140 - protein, 23 aa, Artificial Sequence
  cyclized peptide
  MISC_FEATURE (12)..(12): is D-AA
  MISC_FEATURE (21)..(21): is D-AA
THLETTFTGDGEPKTIRVTQPGQ

SEQ ID NO: 141 - protein, 23 aa, Artificial Sequence
  cyclized peptide
  MISC_FEATURE (21)..(21): is D-AA
THLETTFTGDGDPKTIRVTQPGQ

SEQ ID NO: 142 - protein, 23 aa, Artificial Sequence
  cyclized peptide
  MISC_FEATURE (12)..(12): is D-AA
  MISC_FEATURE (21)..(21): is D-AA
THLETTFTGDGDPKTIRVTQPGQ

SEQ ID NO: 143 - protein, 23 aa, Artificial Sequence
  cyclized peptide
  MISC_FEATURE (12)..(12): X is HomoSer
  MISC_FEATURE (21)..(21): is D-AA
THLETTFTGDGXPKTIRVTQPGQ

SEQ ID NO: 144 - protein, 23 aa, Artificial Sequence
  cyclized peptide
  MISC_FEATURE (12)..(12): X is AlloThr
  MISC_FEATURE (21)..(21): is D-AA
THLETTFTGDGXPKTIRVTQPGQ

SEQ ID NO: 145 - protein, 23 aa, Artificial Sequence
  cyclized peptide
  MISC_FEATURE (12)..(12): X is Glu(OAll)
  MISC_FEATURE (21)..(21): is D-AA
THLETTFTGDGXPKTIRVTQPGQ

SEQ ID NO: 146 - protein, 23 aa, Artificial Sequence
  cyclized peptide
  MISC_FEATURE (12)..(12): X is Cysteic acid
  MISC_FEATURE (21)..(21): is D-AA
THLETTFTGDGXPKTIRVTQPGQ

SEQ ID NO: 147 - protein, 23 aa, Artificial Sequence
  cyclized peptide
  MISC_FEATURE (12)..(12): X is Api
  MISC_FEATURE (21)..(21): is D-AA
THLETTFTGDGXPKTIRVTQPGQ

SEQ ID NO: 148 - protein, 23 aa, Artificial Sequence
  cyclized peptide
  MISC_FEATURE (12)..(12): X is HomoGln
  MISC_FEATURE (21)..(21): is D-AA
THLETTFTGDGXPKTIRVTQPGQ

SEQ ID NO: 149 - protein, 23 aa, Artificial Sequence
  cyclized peptide
  MISC_FEATURE (11)..(11): X is R(Me)
  MISC_FEATURE (21)..(21): is D-AA
THLETTFTGDXTPKTIRVTQPGQ

SEQ ID NO: 150 - protein, 23 aa, Artificial Sequence
  cyclized peptide
  MISC_FEATURE (11)..(11): X is R(Me2)
  MISC_FEATURE (21)..(21): is D-AA
THLETTFTGDXTPKTIRVTQPGQ

SEQ ID NO: 151 - protein, 23 aa, Artificial Sequence
  cyclized peptide
  MISC_FEATURE (21)..(21): is D-AA
THLETTFTGDRTPKTIRVTQPGQ

SEQ ID NO: 152 - protein, 23 aa, Artificial Sequence
  cyclized peptide
  MISC_FEATURE (21)..(21): is D-AA
THLETTFTGDATPKTIRVTQPGQ

SEQ ID NO: 153 - protein, 23 aa, Artificial Sequence
  cyclized peptide
  MISC_FEATURE (21)..(21): is D-AA
THLETTFTGDPTPKTIRVTQPGQ

SEQ ID NO: 154 - protein, 23 aa, Artificial Sequence
  cyclized peptide
  MISC_FEATURE (11)..(11): is D-AA
  MISC_FEATURE (21)..(21): is D-AA
THLETTFTGDATPKTIRVTQPGQ

SEQ ID NO: 155 - protein, 23 aa, Artificial Sequence
  cyclized peptide
  MISC_FEATURE (11)..(11): is D-AA
  MISC_FEATURE (21)..(21): is D-AA
THLETTFTGDPTPKTIRVTQPGQ

SEQ ID NO: 156 - protein, 23 aa, Artificial Sequence
  cyclized peptide
  MISC_FEATURE (10)..(10): is D-AA
  MISC_FEATURE (21)..(21): is D-AA
THLETTFTGDGTPKTIRVTQPGQ

SEQ ID NO: 157 - protein, 23 aa, Artificial Sequence
  cyclized peptide
  MISC_FEATURE (21)..(21): is D-AA
THLETTFTGEGTPKTIRVTQPGQ

SEQ ID NO: 158 - protein, 23 aa, Artificial Sequence
  cyclized peptide
  MISC_FEATURE (10)..(10): is D-AA
  MISC_FEATURE (21)..(21): is D-AA
THLETTFTGEGTPKTIRVTQPGQ

SEQ ID NO: 159 - protein, 23 aa, Artificial Sequence
  cyclized peptide
  MISC_FEATURE (9)..(9): X is D-Cit
  MISC_FEATURE (21)..(21): is D-AA
THLETTFTXDGTPKTIRVTQPGQ

SEQ ID NO: 160 - protein, 23 aa, Artificial Sequence
  cyclized peptide
  MISC_FEATURE (9)..(9): X is Cit
  MISC_FEATURE (21)..(21): is D-AA
THLETTFTXDGTPKTIRVTQPGQ

SEQ ID NO: 161 - protein, 23 aa, Artificial Sequence
  cyclized peptide
  MISC_FEATURE (9)..(9): X is HomoCit
  MISC_FEATURE (21)..(21): is D-AA
THLETTFTXDGTPKTIRVTQPGQ

SEQ ID NO: 162 - protein, 23 aa, Artificial Sequence
  cyclized peptide
  MISC_FEATURE (9)..(9): X is HomoGln
  MISC_FEATURE (21)..(21): is D-AA
THLETTFTXDGTPKTIRVTQPGQ

SEQ ID NO: 163 - protein, 23 aa, Artificial Sequence
  cyclized peptide
  MISC_FEATURE (21)..(21): is D-AA
THLETTFWGDGTPKTIRVTQPGQ

SEQ ID NO: 164 - protein, 23 aa, Artificial Sequence
  cyclized peptide
  MISC_FEATURE (8)..(8): is D-AA
  MISC_FEATURE (21)..(21): is D-AA
THLETTFWGDGTPKTIRVTQPGQ

SEQ ID NO: 165 - protein, 23 aa, Artificial Sequence
  cyclized peptide
  MISC_FEATURE (8)..(8): X is AlloThr
  MISC_FEATURE (21)..(21): is D-AA
THLETTFXGDGTPKTIRVTQPGQ

SEQ ID NO: 166 - protein, 23 aa, Artificial Sequence
  cyclized peptide
  MISC_FEATURE (8)..(8): X is Phe-4-Me
  MISC_FEATURE (21)..(21): is D-AA
THLETTFXGDGTPKTIRVTQPGQ

SEQ ID NO: 167 - protein, 23 aa, Artificial Sequence
  cyclized peptide
  MISC_FEATURE (8)..(8): X is D-Phe-(3,4Cl)
  MISC_FEATURE (21)..(21): is D-AA
THLETTFXGDGTPKTIRVTQPGQ

SEQ ID NO: 168 - protein, 23 aa, Artificial Sequence
  cyclized peptide
  MISC_FEATURE (8)..(8): X is Phe-4-NH2
  MISC_FEATURE (21)..(21): is D-AA
THLETTFXGDGTPKTIRVTQPGQ

SEQ ID NO: 169 - protein, 23 aa, Artificial Sequence
  cyclized peptide
  MISC_FEATURE (8)..(8): X is BIP
  MISC_FEATURE (21)..(21): is D-AA
THLETTFXGDGTPKTIRVTQPGQ

SEQ ID NO: 170 - protein, 23 aa, Artificial Sequence
  cyclized peptide
  MISC_FEATURE (8)..(8): X is D-HomoCha
  MISC_FEATURE (21)..(21): is D-AA
THLETTFXGDGTPKTIRVTQPGQ

SEQ ID NO: 171 - protein, 23 aa, Artificial Sequence
  cyclized peptide
  MISC_FEATURE (8)..(8): X is HomoCha
  MISC_FEATURE (21)..(21): is D-AA
THLETTFXGDGTPKTIRVTQPGQ

SEQ ID NO: 172 - protein, 23 aa, Artificial Sequence
  cyclized peptide
  MISC_FEATURE (8)..(8): X is Cha
  MISC_FEATURE (21)..(21): is D-AA
THLETTFXGDGTPKTIRVTQPGQ

SEQ ID NO: 173 - protein, 23 aa, Artificial Sequence
  cyclized peptide
  MISC_FEATURE (8)..(8): X is D-Cha
  MISC_FEATURE (21)..(21): is D-AA
THLETTFXGDGTPKTIRVTQPGQ

SEQ ID NO: 174 - protein, 23 aa, Artificial Sequence
  cyclized peptide
  MISC_FEATURE (21)..(21): is D-AA
THLETTFYGDGTPKTIRVTQPGQ

SEQ ID NO: 175 - protein, 23 aa, Artificial Sequence
  cyclized peptide
  MISC_FEATURE (8)..(8): is D-AA
  MISC_FEATURE (21)..(21): is D-AA
THLETTFYGDGTPKTIRVTQPGQ

SEQ ID NO: 176 - protein, 23 aa, Artificial Sequence
  cyclized peptide
  MISC_FEATURE (8)..(8): X is D-Tyr-2-(Et)Cha
  MISC_FEATURE (21)..(21): is D-AA
THLETTFXGDGTPKTIRVTQPGQ

SEQ ID NO: 177 - protein, 23 aa, Artificial Sequence
  cyclized peptide
  MISC_FEATURE (8)..(8): X is D-Cit
  MISC_FEATURE (21)..(21): is D-AA
THLETTFXGDGTPKTIRVTQPGQ

SEQ ID NO: 178 - protein, 23 aa, Artificial Sequence
  cyclized peptide
  MISC_FEATURE (8)..(8): X is Cit
  MISC_FEATURE (21)..(21): is D-AA
THLETTFXGDGTPKTIRVTQPGQ

SEQ ID NO: 179 - protein, 23 aa, Artificial Sequence
  cyclized peptide
  MISC_FEATURE (8)..(8): X is D-Dap
  MISC_FEATURE (21)..(21): is D-AA
THLETTFXGDGTPKTIRVTQPGQ

SEQ ID NO: 180 - protein, 23 aa, Artificial Sequence
  cyclized peptide
  MISC_FEATURE (8)..(8): X is Dap
  MISC_FEATURE (21)..(21): is D-AA
THLETTFXGDGTPKTIRVTQPGQ

SEQ ID NO: 181 - protein, 23 aa, Artificial Sequence
  cyclized peptide
  MISC_FEATURE (8)..(8): X is Nal2
  MISC_FEATURE (21)..(21): is D-AA
THLETTFXGDGTPKTIRVTQPGQ

SEQ ID NO: 182 - protein, 23 aa, Artificial Sequence
  cyclized peptide
  MISC_FEATURE (8)..(8): X is IndaG
  MISC_FEATURE (21)..(21): is D-AA
THLETTFXGDGTPKTIRVTQPGQ

SEQ ID NO: 183 - protein, 23 aa, Artificial Sequence
  cyclized peptide
  MISC_FEATURE (8)..(8): X is Trp-2-Me
  MISC_FEATURE (21)..(21): is D-AA
THLETTFXGDGTPKTIRVTQPGQ

SEQ ID NO: 184 - protein, 23 aa, Artificial Sequence
  cyclized peptide
  MISC_FEATURE (8)..(8): X is Aic
  MISC_FEATURE (21)..(21): is D-AA
THLETTFXGDGTPKTIRVTQPGQ

SEQ ID NO: 185 - protein, 23 aa, Artificial Sequence
  cyclized peptide
  MISC_FEATURE (21)..(21): is D-AA
THLETTFFGDGTPKTIRVTQPGQ

SEQ ID NO: 186 - protein, 23 aa, Artificial Sequence
  cyclized peptide
  MISC_FEATURE (8)..(8): is D-AA
  MISC_FEATURE (21)..(21): is D-AA
THLETTFFGDGTPKTIRVTQPGQ

SEQ ID NO: 187 - protein, 23 aa, Artificial Sequence
  cyclized peptide
  MISC_FEATURE (21)..(21): is D-AA
THLETTFHGDGTPKTIRVTQPGQ

SEQ ID NO: 188 - protein, 23 aa, Artificial Sequence
  cyclized peptide
  MISC_FEATURE (8)..(8): X is HomoPhe
  MISC_FEATURE (21)..(21): is D-AA
THLETTFXGDGTPKTIRVTQPGQ

SEQ ID NO: 189 - protein, 23 aa, Artificial Sequence
  cyclized peptide
  MISC_FEATURE (8)..(8): X is Napthoic acid
  MISC_FEATURE (21)..(21): is D-AA
THLETTFXGDGTPKTIRVTQPGQ

SEQ ID NO: 190 - protein, 23 aa, Artificial Sequence
  cyclized peptide
  MISC_FEATURE (7)..(7): X is Cha
  MISC_FEATURE (21)..(21): is D-AA
THLETTXTGDGTPKTIRVTQPGQ

SEQ ID NO: 191 - protein, 26 aa, Artificial Sequence
  cyclized peptide
  MISC_FEATURE (7)..(7): X is D-Cha
  MISC_FEATURE (21)..(21): is D-AA
THLETTDCHATGDGTPKTIRVTQPGQ

SEQ ID NO: 192 - protein, 22 aa, Artificial Sequence
  cyclized peptide
  MISC_FEATURE (7)..(7): X is D-HomoCha
  MISC_FEATURE (21)..(21): is D-AA
THLETTXGDGTPKTIRVTQPGQ

SEQ ID NO: 193 - protein, 23 aa, Artificial Sequence
  cyclized peptide
  MISC_FEATURE (7)..(7): X is HomoCha
  MISC_FEATURE (21)..(21): is D-AA
THLETTXTGDGTPKTIRVTQPGQ

SEQ ID NO: 194 - protein, 23 aa, Artificial Sequence
  cyclized peptide
  MISC_FEATURE (7)..(7): X is D-HomoPhe
  MISC_FEATURE (21)..(21): is D-AA
THLETTXTGDGTPKTIRVTQPGQ

SEQ ID NO: 195 - protein, 23 aa, Artificial Sequence
  cyclized peptide
  MISC_FEATURE (7)..(7): X is Phg
  MISC_FEATURE (21)..(21): is D-AA
THLETTXTGDGTPKTIRVTQPGQ

SEQ ID NO: 196 - protein, 23 aa, Artificial Sequence
  cyclized peptide
  MISC_FEATURE (7)..(7): X is Nal1
  MISC_FEATURE (21)..(21): is D-AA
THLETTXTGDGTPKTIRVTQPGQ

SEQ ID NO: 197 - protein, 23 aa, Artificial Sequence
  cyclized peptide
  MISC_FEATURE (7)..(7): X is Nal2
  MISC_FEATURE (21)..(21): is D-AA
THLETTXTGDGTPKTIRVTQPGQ

SEQ ID NO: 198 - protein, 23 aa, Artificial Sequence
  cyclized peptide
  MISC_FEATURE (7)..(7): X is StyAla
  MISC_FEATURE (21)..(21): is D-AA
THLETTXTGDGTPKTIRVTQPGQ

SEQ ID NO: 199 - protein, 23 aa, Artificial Sequence
  cyclized peptide
  MISC_FEATURE (7)..(7): X is Phe-4-Me
  MISC_FEATURE (21)..(21): is D-AA
THLETTXTGDGTPKTIRVTQPGQ

SEQ ID NO: 200 - protein, 23 aa, Artificial Sequence
  cyclized peptide
  MISC_FEATURE (7)..(7): X is Phe-2-Me
  MISC_FEATURE (21)..(21): is D-AA
THLETTXTGDGTPKTIRVTQPGQ

SEQ ID NO: 201 - protein, 23 aa, Artificial Sequence
  cyclized peptide
  MISC_FEATURE (7)..(7): X is Phe-4-tBu
  MISC_FEATURE (21)..(21): is D-AA
THLETTXTGDGTPKTIRVTQPGQ

SEQ ID NO: 202 - protein, 23 aa, Artificial Sequence
  cyclized peptide
  MISC_FEATURE (7)..(7): X is Phe-4-F
  MISC_FEATURE (21)..(21): is D-AA
THLETTXTGDGTPKTIRVTQPGQ

SEQ ID NO: 203 - protein, 23 aa, Artificial Sequence
  cyclized peptide
  MISC_FEATURE (7)..(7): X is Phe-3-F
  MISC_FEATURE (21)..(21): is D-AA
THLETTXTGDGTPKTIRVTQPGQ

SEQ ID NO: 204 - protein, 23 aa, Artificial Sequence
  cyclized peptide
  MISC_FEATURE (7)..(7): X is Phe-2-F
  MISC_FEATURE (21)..(21): is D-AA
THLETTXTGDGTPKTIRVTQPGQ

SEQ ID NO: 205 - protein, 23 aa, Artificial Sequence
  cyclized peptide
  MISC_FEATURE (7)..(7): X is Phe-4-Cl
  MISC_FEATURE (21)..(21): is D-AA
THLETTXTGDGTPKTIRVTQPGQ

SEQ ID NO: 206 - protein, 23 aa, Artificial Sequence
  cyclized peptide
  MISC_FEATURE (7)..(7): X is Phe-3-Cl
  MISC_FEATURE (21)..(21): is D-AA
THLETTXTGDGTPKTIRVTQPGQ

SEQ ID NO: 207 - protein, 23 aa, Artificial Sequence
  cyclized peptide
  MISC_FEATURE (7)..(7): X is Phe-2-Cl
  MISC_FEATURE (21)..(21): is D-AA
THLETTXTGDGTPKTIRVTQPGQ

SEQ ID NO: 208 - protein, 23 aa, Artificial Sequence
  cyclized peptide
  MISC_FEATURE (7)..(7): X is Phe-4-Br
  MISC_FEATURE (21)..(21): is D-AA
THLETTXTGDGTPKTIRVTQPGQ

SEQ ID NO: 209 - protein, 23 aa, Artificial Sequence
  cyclized peptide
  MISC_FEATURE (7)..(7): X is Phe-3-Br
  MISC_FEATURE (21)..(21): is D-AA
THLETTXTGDGTPKTIRVTQPGQ

SEQ ID NO: 210 - protein, 23 aa, Artificial Sequence
  cyclized peptide
  MISC_FEATURE (7)..(7): X is Phe-2-Br
  MISC_FEATURE (21)..(21): is D-AA
THLETTXTGDGTPKTIRVTQPGQ

SEQ ID NO: 211 - protein, 23 aa, Artificial Sequence
  cyclized peptide
  MISC_FEATURE (7)..(7): is D-AA
  MISC_FEATURE (21)..(21): is D-AA
THLETTFTGDGTPKTIRVTQPGQ

SEQ ID NO: 212 - protein, 23 aa, Artificial Sequence
  cyclized peptide
  MISC_FEATURE (7)..(7): X is BIP
  MISC_FEATURE (21)..(21): is D-AA
THLETTXTGDGTPKTIRVTQPGQ

SEQ ID NO: 213 - protein, 23 aa, Artificial Sequence
  cyclized peptide
  MISC_FEATURE (7)..(7): X is Phe-4-Me
  MISC_FEATURE (21)..(21): is D-AA
THLETTXTGDGTPKTIRVTQPGQ

SEQ ID NO: 214 - protein, 23 aa, Artificial Sequence
  cyclized peptide
  MISC_FEATURE (21)..(21): is D-AA
THLETEFTGDGTPKTIRVTQPGQ

SEQ ID NO: 215 - protein, 23 aa, Artificial Sequence
  cyclized peptide
  MISC_FEATURE (21)..(21): is D-AA
THLETDFTGDGTPKTIRVTQPGQ

SEQ ID NO: 216 - protein, 23 aa, Artificial Sequence
  cyclized peptide
  MISC_FEATURE (6)..(6): X is Cysteic Acid
  MISC_FEATURE (21)..(21): is D-AA
THLETXFTGDGTPKTIRVTQPGQ

SEQ ID NO: 217 - protein, 23 aa, Artificial Sequence
  cyclized peptide
  MISC_FEATURE (6)..(6): X is Api
  MISC_FEATURE (21)..(21): is D-AA
THLETXFTGDGTPKTIRVTQPGQ

SEQ ID NO: 218 - protein, 23 aa, Artificial Sequence
  cyclized peptide
  MISC_FEATURE (6)..(6): is D-AA
  MISC_FEATURE (21)..(21): is D-AA
THLETEFTGDGTPKTIRVTQPGQ

SEQ ID NO: 219 - protein, 23 aa, Artificial Sequence
  cyclized peptide
  MISC_FEATURE (6)..(6): is D-AA
  MISC_FEATURE (21)..(21): is D-AA
THLETDFTGDGTPKTIRVTQPGQ

SEQ ID NO: 220 - protein, 23 aa, Artificial Sequence
  cyclized peptide
  MISC_FEATURE (6)..(6): is D-AA
  MISC_FEATURE (21)..(21): is D-AA
THLETTFTGDGTPKTIRVTQPGQ

SEQ ID NO: 221 - protein, 23 aa, Artificial Sequence
  cyclized peptide
  MISC_FEATURE (6)..(6): X is AlloThr
  MISC_FEATURE (21)..(21): is D-AA
THLETXFTGDGTPKTIRVTQPGQ

SEQ ID NO: 222 - protein, 23 aa, Artificial Sequence
  cyclized peptide
  MISC_FEATURE (5)..(5): X is AlloThr
  MISC_FEATURE (21)..(21): is D-AA
THLEXTFTGDGTPKTIRVTQPGQ

SEQ ID NO: 223 - protein, 23 aa, Artificial Sequence
  cyclized peptide
  MISC_FEATURE (5)..(5): is D-AA
  MISC_FEATURE (21)..(21): is D-AA
THLETTFTGDGTPKTIRVTQPGQ

SEQ ID NO: 224 - protein, 23 aa, Artificial Sequence
  cyclized peptide
  MISC_FEATURE (4)..(4): X is Glu(OAll)
  MISC_FEATURE (21)..(21): is D-AA
THLXTTFTGDGTPKTIRVTQPGQ

SEQ ID NO: 225 - protein, 23 aa, Artificial Sequence
  cyclized peptide
  MISC_FEATURE (4)..(4): X is Orn
  MISC_FEATURE (21)..(21): is D-AA
THLXTTFTGDGTPKTIRVTQPGQ

SEQ ID NO: 226 - protein, 23 aa, Artificial Sequence
  cyclized peptide
  MISC_FEATURE (4)..(4): X is Cysteic Acid
  MISC_FEATURE (21)..(21): is D-AA
THLXTTFTGDGTPKTIRVTQPGQ

SEQ ID NO: 227 - protein, 23 aa, Artificial Sequence
  cyclized peptide
  MISC_FEATURE (21)..(21): is D-AA
THLDTTFTGDGTPKTIRVTQPGQ

SEQ ID NO: 228 - protein, 23 aa, Artificial Sequence
  cyclized peptide
  MISC_FEATURE (4)..(4): is D-AA
  MISC_FEATURE (21)..(21): is D-AA
THLETTFTGDGTPKTIRVTQPGQ

SEQ ID NO: 229 - protein, 23 aa, Artificial Sequence
  cyclized peptide
  MISC_FEATURE (4)..(4): is D-AA
  MISC_FEATURE (21)..(21): is D-AA
THLDTTFTGDGTPKTIRVTQPGQ

SEQ ID NO: 230 - protein, 23 aa, Artificial Sequence
  cyclized peptide
  MISC_FEATURE (21)..(21): is D-AA
THLQTTFTGDGTPKTIRVTQPGQ

SEQ ID NO: 231 - protein, 23 aa, Artificial Sequence
  cyclized peptide
  MISC_FEATURE (4)..(4): X is HomoGln
  MISC_FEATURE (21)..(21): is D-AA
THLXTTFTGDGTPKTIRVTQPGQ

SEQ ID NO: 232 - protein, 23 aa, Artificial Sequence
  cyclized peptide
  misc (4)..(4): X is HomoSer
  misc (21)..(21): is D-AA
THLXTTFTGDGTPKTIRVTQPGQ

SEQ ID NO: 233 - protein, 23 aa, Artificial Sequence
  cyclized peptide
  MISC_FEATURE (3)..(3): X is HomoCha
  MISC_FEATURE (21)..(21): is D-AA
THXETTFTGDGTPKTIRVTQPGQ

SEQ ID NO: 234 - protein, 23 aa, Artificial Sequence
  cyclized peptide
  MISC_FEATURE (3)..(3): X is Cha
THXETTFTGDGTPKTIRVTQPGQ

SEQ ID NO: 235 - protein, 23 aa, Artificial Sequence
  cyclized peptide
  MISC_FEATURE (3)..(3): X is D-Orn
  MISC_FEATURE (21)..(21): is D-AA
THXETTFTGDGTPKTIRVTQPGQ

SEQ ID NO: 236 - protein, 23 aa, Artificial Sequence
  cyclized peptide
  MISC_FEATURE (3)..(3): X is HomoLeu
  MISC_FEATURE (21)..(21): is D-AA
THXETTFTGDGTPKTIRVTQPGQ

SEQ ID NO: 237 - protein, 23 aa, Artificial Sequence
  cyclized peptide
  MISC_FEATURE (3)..(3): X is NLeu
  MISC_FEATURE (21)..(21): is D-AA
THXETTFTGDGTPKTIRVTQPGQ

SEQ ID NO: 238 - protein, 23 aa, Artificial Sequence
  cyclized peptide
  MISC_FEATURE (3)..(3): X is CycLeu
  MISC_FEATURE (21)..(21): is D-AA
THXETTFTGDGTPKTIRVTQPGQ

SEQ ID NO: 239 - protein, 23 aa, Artificial Sequence
  cyclized peptide
  MISC_FEATURE (3)..(3): X is Aib
  MISC_FEATURE (21)..(21): is D-AA
THXETTFTGDGTPKTIRVTQPGQ

SEQ ID NO: 240 - protein, 23 aa, Artificial Sequence
  cyclized peptide
  MISC_FEATURE (3)..(3): X is Abu
  MISC_FEATURE (21)..(21): is D-AA
THXETTFTGDGTPKTIRVTQPGQ

SEQ ID NO: 241 - protein, 23 aa, Artificial Sequence
  cyclized peptide
  MISC_FEATURE (21)..(21): is D-AA
THVETTFTGDGTPKTIRVTQPGQ

SEQ ID NO: 242 - protein, 23 aa, Artificial Sequence
  cyclized peptide
  MISC_FEATURE (3)..(3): X is NVa
  MISC_FEATURE (21)..(21): is D-AA
THXETTFTGDGTPKTIRVTQPGQ

SEQ ID NO: 243 - protein, 23 aa, Artificial Sequence
  cyclized peptide
  MISC_FEATURE (3)..(3): X is Tle
  MISC_FEATURE (21)..(21): is D-AA
THXETTFTGDGTPKTIRVTQPGQ

SEQ ID NO: 244 - protein, 23 aa, Artificial Sequence
  cyclized peptide
  MISC_FEATURE (3)..(3): is D-AA
  MISC_FEATURE (21)..(21): is D-AA
THVETTFTGDGTPKTIRVTQPGQ

SEQ ID NO: 245 - protein, 23 aa, Artificial Sequence
  cyclized peptide
  MISC_FEATURE (3)..(3): X is D-HomoCha
  MISC_FEATURE (21)..(21): is D-AA
THXETTFTGDGTPKTIRVTQPGQ

SEQ ID NO: 246 - protein, 23 aa, Artificial Sequence
  cyclized peptide
  MISC_FEATURE (21)..(21): is D-AA
THIETTFTGDGTPKTIRVTQPGQ

SEQ ID NO: 247 - protein, 23 aa, Artificial Sequence
  cyclized peptide
  MISC_FEATURE (2)..(2): X is Phe-4-CH2NH2
  MISC_FEATURE (21)..(21): is D-AA
TXLETTFTGDGTPKTIRVTQPGQ

SEQ ID NO: 248 - protein, 23 aa, Artificial Sequence
  cyclized peptide
  MISC_FEATURE (2)..(2): X is Phe-3-CH2NH2
  MISC_FEATURE (21)..(21): is D-AA
TXLETTFTGDGTPKTIRVTQPGQ

SEQ ID NO: 249 - protein, 23 aa, Artificial Sequence
  cyclized peptide
  MISC_FEATURE (2)..(2): X is Phe-4-NH2
  MISC_FEATURE (21)..(21): is D-AA
TXLETTFTGDGTPKTIRVTQPGQ

SEQ ID NO: 250 - protein, 23 aa, Artificial Sequence
  cyclized peptide
  MISC_FEATURE (2)..(2): X is Phe-4-N3
  MISC_FEATURE (21)..(21): is D-AA
TXLETTFTGDGTPKTIRVTQPGQ

SEQ ID NO: 251 - protein, 23 aa, Artificial Sequence
  cyclized peptide
  MISC_FEATURE (2)..(2): X is PyA-3
  MISC_FEATURE (21)..(21): is D-AA
TXLETTFTGDGTPKTIRVTQPGQ

SEQ ID NO: 252 - protein, 25 aa, Artificial Sequence
  cyclized peptide
  MISC_FEATURE (2)..(2): X is PyA-4
  MISC_FEATURE (21)..(21): is D-AA
TPYALETTFTGDGTPKTIRVTQPGQ

SEQ ID NO: 253 - protein, 23 aa, Artificial Sequence
  cyclized peptide
  MISC_FEATURE (2)..(2): X is FurAla
  MISC_FEATURE (21)..(21): is D-AA
TXLETTFTGDGTPKTIRVTQPGQ

SEQ ID NO: 254 - protein, 23 aa, Artificial Sequence
  cyclized peptide
  MISC_FEATURE (2)..(2): X is Cha
  MISC_FEATURE (21)..(21): is D-AA
TXLETTFTGDGTPKTIRVTQPGQ

SEQ ID NO: 255 - protein, 23 aa, Artificial Sequence
  cyclized peptide
  MISC_FEATURE (2)..(2): X is Thi
  MISC_FEATURE (21)..(21): is D-AA
TXLETTFTGDGTPKTIRVTQPGQ

SEQ ID NO: 256 - protein, 23 aa, Artificial Sequence
  cyclized peptide
  MISC_FEATURE (2)..(2): X is His-1Me
  MISC_FEATURE (21)..(21): is D-AA
TXLETTFTGDGTPKTIRVTQPGQ

SEQ ID NO: 257 - protein, 23 aa, Artificial Sequence
  cyclized peptide
  MISC_FEATURE (2)..(2): X is His-3Me
  MISC_FEATURE (21)..(21): is D-AA
TXLETTFTGDGTPKTIRVTQPGQ

SEQ ID NO: 258 - protein, 23 aa, Artificial Sequence
  cyclized peptide
  MISC_FEATURE (21)..(21): is D-AA
TKLETTFTGDGTPKTIRVTQPGQ

SEQ ID NO: 259 - protein, 23 aa, Artificial Sequence
  cyclized peptide
  MISC_FEATURE (2)..(2): X is HomoCit
  MISC_FEATURE (21)..(21): is D-AA
TXLETTFTGDGTPKTIRVTQPGQ

SEQ ID NO: 260 - protein, 23 aa, Artificial Sequence
  cyclized peptide
  MISC_FEATURE (2)..(2): X is Orn
  MISC_FEATURE (21)..(21): is D-AA
TXLETTFTGDGTPKTIRVTQPGQ

SEQ ID NO: 261 - protein, 23 aa, Artificial Sequence
  cyclized peptide
  MISC_FEATURE (2)..(2): X is HomoLys
  MISC_FEATURE (21)..(21): is D-AA
TXLETTFTGDGTPKTIRVTQPGQ

SEQ ID NO: 262 - protein, 23 aa, Artificial Sequence
  cyclized peptide
  MISC_FEATURE (21)..(21): is D-AA
TRLETTFTGDGTPKTIRVTQPGQ

SEQ ID NO: 263 - protein, 23 aa, Artificial Sequence
  cyclized peptide
  MISC_FEATURE (21)..(21): is D-AA
TYLETTFTGDGTPKTIRVTQPGQ

SEQ ID NO: 264 - protein, 23 aa, Artificial Sequence
  cyclized peptide
  MISC_FEATURE (2)..(2): X is Apg
  MISC_FEATURE (21)..(21): is D-AA
TXLETTFTGDGTPKTIRVTQPGQ

SEQ ID NO: 265 - protein, 23 aa, Artificial Sequence
  cyclized peptide
  MISC_FEATURE (2)..(2): X is HomoArg
  MISC_FEATURE (21)..(21): is D-AA
TXLETTFTGDGTPKTIRVTQPGQ

SEQ ID NO: 266 - protein, 23 aa, Artificial Sequence
  cyclized peptide
  MISC_FEATURE (1)..(1): X is Phe-4-Cl
  MISC_FEATURE (21)..(21): is D-AA
XHLETTFTGDGTPKTIRVTQPGQ

SEQ ID NO: 267 - protein, 23 aa, Artificial Sequence
  cyclized peptide
  MISC_FEATURE (1)..(1): X is Phe-3-Cl
  MISC_FEATURE (21)..(21): is D-AA
XHLETTFTGDGTPKTIRVTQPGQ

SEQ ID NO: 268 - protein, 23 aa, Artificial Sequence
  cyclized peptide
  MISC_FEATURE (1)..(1): X is Phe-2-Cl
  MISC_FEATURE (21)..(21): is D-AA
XHLETTFTGDGTPKTIRVTQPGQ

SEQ ID NO: 269 - protein, 23 aa, Artificial Sequence
  cyclized peptide
  MISC_FEATURE (1)..(1): X is D-Phe-4-NH2
  MISC_FEATURE (21)..(21): is D-AA
XHLETTFTGDGTPKTIRVTQPGQ

SEQ ID NO: 270 - protein, 23 aa, Artificial Sequence
  cyclized peptide
  MISC_FEATURE (1)..(1): X is HomoPhe
  MISC_FEATURE (21)..(21): is D-AA
XHLETTFTGDGTPKTIRVTQPGQ

SEQ ID NO: 271 - protein, 23 aa, Artificial Sequence
  cyclized peptide
  MISC_FEATURE (21)..(21): is D-AA
KHLETTFTGDGTPKTIRVTQPGQ

SEQ ID NO: 272 - protein, 23 aa, Artificial Sequence
  cyclized peptide
  MISC_FEATURE (1)..(1): is D-AA
  MISC_FEATURE (21)..(21): is D-AA
HHLETTFTGDGTPKTIRVTQPGQ

SEQ ID NO: 273 - protein, 23 aa, Artificial Sequence
  cyclized peptide
  MISC_FEATURE (1)..(1): X is Phe-4-NH2
  MISC_FEATURE (21)..(21): is D-AA
XHLETTFTGDGTPKTIRVTQPGQ

SEQ ID NO: 274 - protein, 23 aa, Artificial Sequence
  cyclized peptide
  MISC_FEATURE (1)..(1): X is AlloThr
  MISC_FEATURE (21)..(21): is D-AA
XHLETTFTGDGTPKTIRVTQPGQ

SEQ ID NO: 275 - protein, 23 aa, Artificial Sequence
  cyclized peptide
  MISC_FEATURE (1)..(1): is D-AA
  MISC_FEATURE (21)..(21): is D-AA
THLETTFTGDGTPKTIRVTQPGQ

SEQ ID NO: 276 - protein, 23 aa, Artificial Sequence
  cyclized peptide
  MISC_FEATURE (21)..(21): is D-AA
RHLETTFTGDGTPKTIRVTQPGQ

SEQ ID NO: 277 - protein, 23 aa, Artificial Sequence
  cyclized peptide
  MISC_FEATURE (21)..(21): is D-AA
FHLETTFTGDGTPKTIRVTQPGQ

SEQ ID NO: 278 - protein, 23 aa, Artificial Sequence
  cyclized peptide
  MISC_FEATURE (21)..(21): is D-AA
YHLETTFTGDGTPKTIRVTQPGQ

SEQ ID NO: 279 - protein, 23 aa, Artificial Sequence
  cyclized peptide
  MISC_FEATURE (12)..(12): X is HomoSer
  MISC_FEATURE (21)..(21): X is HomoSer
THLETTFWGDGXPKTIRVTQPGQ

SEQ ID NO: 280 - protein, 23 aa, Artificial Sequence
  cyclized peptide
  MISC_FEATURE (12)..(12): X is HomoGln
  MISC_FEATURE (21)..(21): is D-AA
THLETTFWGDGXPKTIRVTQPGQ

SEQ ID NO: 281 - protein, 23 aa, Artificial Sequence
  cyclized peptide
  MISC_FEATURE (12)..(12): X is Glu(OAll)
  MISC_FEATURE (21)..(21): is D-AA
THLETTFWGDGXPKTIRVTQPGQ

SEQ ID NO: 282 - protein, 23 aa, Artificial Sequence
  cyclized peptide
  MISC_FEATURE (12)..(12): is D-AA
  MISC_FEATURE (21)..(21): is D-AA
THLETTFWGDGEPKTIRVTQPGQ

SEQ ID NO: 283 - protein, 23 aa, Artificial Sequence
  cyclized peptide
  MISC_FEATURE (21)..(21): is D-AA
THLETTFWGDGDPKTIRVTQPGQ

SEQ ID NO: 284 - protein, 23 aa, Artificial Sequence
  cyclized peptide
  MISC_FEATURE (12)..(12): is D-AA
  MISC_FEATURE (21)..(21): is D-AA
THLETTFWGDGDPKTIRVTQPGQ

SEQ ID NO: 285 - protein, 23 aa, Artificial Sequence
  cyclized peptide
  MISC_FEATURE (12)..(12): X is Cysteic acid
  MISC_FEATURE (21)..(21): is D-AA
THLETTFWGDGXPKTIRVTQPGQ

SEQ ID NO: 286 - protein, 23 aa, Artificial Sequence
  cyclized peptide
  MISC_FEATURE (12)..(12): X is Api
  MISC_FEATURE (21)..(21): is D-AA
THLETTFWGDGXPKTIRVTQPGQ

SEQ ID NO: 287 - protein, 23 aa, Artificial Sequence
  cyclized peptide
  MISC_FEATURE (12)..(12): X is AlloThr
  MISC_FEATURE (21)..(21): is D-AA
THLETTFWGDGXPKTIRVTQPGQ

SEQ ID NO: 288 - protein, 23 aa, Artificial Sequence
  cyclized peptide
  MISC_FEATURE (21)..(21): is D-AA
THLETTFYGDGEPKTIRVTQPGQ

SEQ ID NO: 289 - protein, 23 aa, Artificial Sequence
  cyclized peptide
  MISC_FEATURE (8)..(8): is D-AA
  MISC_FEATURE (21)..(21): is D-AA
THLETTFYGDGEPKTIRVTQPGQ

SEQ ID NO: 290 - protein, 23 aa, Artificial Sequence
  cyclized peptide
  MISC_FEATURE (8)..(8): is D-AA
  MISC_FEATURE (21)..(21): is D-AA
THLETTFWGDGEPKTIRVTQPGQ

SEQ ID NO: 291 - protein, 24 aa, Artificial Sequence
  cyclized peptide
  MISC_FEATURE (8)..(8): X is Naphtoic acid
  MISC_FEATURE (21)..(21): is D-AA
THLETTFXGDGEPKTIRVTQPGQC

SEQ ID NO: 292 - protein, 23 aa, Artificial Sequence
  cyclized peptide
  MISC_FEATURE (8)..(8): X is Nal2
  MISC_FEATURE (21)..(21): is D-AA
THLETTFXGDGEPKTIRVTQPGQ

SEQ ID NO: 293 - protein, 23 aa, Artificial Sequence
  cyclized peptide
  MISC_FEATURE (8)..(8): X is Nal1
  MISC_FEATURE (21)..(21): is D-AA
THLETTFXGDGEPKTIRVTQPGQ

SEQ ID NO: 294 - protein, 23 aa, Artificial Sequence
  cyclized peptide
  MISC_FEATURE (8)..(8): X is IndaG
  MISC_FEATURE (21)..(21): is D-AA
THLETTFXGDGEPKTIRVTQPGQ

SEQ ID NO: 295 - protein, 23 aa, Artificial Sequence
  cyclized peptide
  MISC_FEATURE (8)..(8): X is HomoCha
  MISC_FEATURE (21)..(21): is D-AA
THLETTFXGDGEPKTIRVTQPGQ

SEQ ID NO: 296 - protein, 23 aa, Artificial Sequence
  cyclized peptide
  MISC_FEATURE (8)..(8): X is Phe-4-NH2
  MISC_FEATURE (21)..(21): is D-AA
THLETTFXGDGEPKTIRVTQPGQ

SEQ ID NO: 297 - protein, 23 aa, Artificial Sequence
  cyclized peptide
  MISC_FEATURE (8)..(8): X is Dap
  MISC_FEATURE (21)..(21): is D-AA
THLETTFXGDGEPKTIRVTQPGQ

SEQ ID NO: 298 - protein, 23 aa, Artificial Sequence
  cyclized peptide
  MISC_FEATURE (8)..(8): X is HomoPhe
  MISC_FEATURE (21)..(21): is D-AA
THLETTFXGDGEPKTIRVTQPGQ

SEQ ID NO: 299 - protein, 23 aa, Artificial Sequence
  cyclized peptide
  MISC_FEATURE (21)..(21): is D-AA
THLETTFHGDGEPKTIRVTQPGQ

SEQ ID NO: 300 - protein, 23 aa, Artificial Sequence
  cyclized peptide
  MISC_FEATURE (21)..(21): is D-AA
THLETTFFGDGEPKTIRVTQPGQ

SEQ ID NO: 301 - protein, 23 aa, Artificial Sequence
  cyclized peptide
  MISC_FEATURE (8)..(8): is D-AA
  MISC_FEATURE (21)..(21): is D-AA
THLETTFFGDGEPKTIRVTQPGQ

SEQ ID NO: 302 - protein, 23 aa, Artificial Sequence
  cyclized peptide
  MISC_FEATURE (8)..(8): X is D-HomoCha
  MISC_FEATURE (21)..(21): is D-AA
THLETTFXGDGEPKTIRVTQPGQ

SEQ ID NO: 303 - protein, 23 aa, Artificial Sequence
  cyclized peptide
  MISC_FEATURE (8)..(8): X is D-Dap
  MISC_FEATURE (21)..(21): is D-AA
THLETTFXGDGEPKTIRVTQPGQ

SEQ ID NO: 304 - protein, 23 aa, Artificial Sequence
  cyclized peptide
  MISC_FEATURE (8)..(8): X is D-Cit
  MISC_FEATURE (21)..(21): is D-AA
THLETTFXGDGEPKTIRVTQPGQ

SEQ ID NO: 305 - protein, 23 aa, Artificial Sequence
  cyclized peptide
  MISC_FEATURE (8)..(8): X is D-Cha
  MISC_FEATURE (21)..(21): is D-AA
THLETTFXGDGEPKTIRVTQPGQ

SEQ ID NO: 306 - protein, 23 aa, Artificial Sequence
  cyclized peptide
  MISC_FEATURE (8)..(8): X is D-Tyr(Et)
  MISC_FEATURE (21)..(21): is D-AA
THLETTFXGDGEPKTIRVTQPGQ

SEQ ID NO: 307 - protein, 23 aa, Artificial Sequence
  cyclized peptide
  MISC_FEATURE (8)..(8): X is Cit
  MISC_FEATURE (21)..(21): is D-AA
THLETTFXGDGEPKTIRVTQPGQ

SEQ ID NO: 308 - protein, 23 aa, Artificial Sequence
  cyclized peptide
  MISC_FEATURE (8)..(8): X is Cha
  MISC_FEATURE (21)..(21): is D-AA
THLETTFXGDGEPKTIRVTQPGQ

SEQ ID NO: 309 - protein, 23 aa, Artificial Sequence
  cyclized peptide
  MISC_FEATURE (8)..(8): X is BIP
  MISC_FEATURE (21)..(21): is D-AA
THLETTFXGDGEPKTIRVTQPGQ

SEQ ID NO: 310 - protein, 23 aa, Artificial Sequence
  cyclized peptide
  MISC_FEATURE (8)..(8): X is AllThr
  MISC_FEATURE (21)..(21): is D-AA
THLETTFXGDGEPKTIRVTQPGQ

SEQ ID NO: 311 - protein, 23 aa, Artificial Sequence
  cyclized peptide
  MISC_FEATURE (8)..(8): X is Aic
  MISC_FEATURE (21)..(21): is D-AA
THLETTFXGDGEPKTIRVTQPGQ

SEQ ID NO: 312 - protein, 23 aa, Artificial Sequence
  cyclized peptide
  MISC_FEATURE (8)..(8): X is Phe-2-Me
  MISC_FEATURE (21)..(21): is D-AA
THLETTFXGDGEPKTIRVTQPGQ

SEQ ID NO: 313 - protein, 23 aa, Artificial Sequence
  cyclized peptide
  MISC_FEATURE (8)..(8): X is D-Phe-2-(3,4Cl)
  MISC_FEATURE (21)..(21): is D-AA
THLETTFXGDGEPKTIRVTQPGQ

SEQ ID NO: 314 - protein, 23 aa, Artificial Sequence
  cyclized peptide
  MISC_FEATURE (8)..(8): X is Trp-2-Me
  MISC_FEATURE (21)..(21): is D-AA
THLETTFXGDGEPKTIRVTQPGQ

SEQ ID NO: 315 - protein, 23 aa, Artificial Sequence
  cyclized peptide
  MISC_FEATURE (6)..(6): is D-AA
  MISC_FEATURE (21)..(21): is D-AA
THLETTFWGDGEPKTIRVTQPGQ

SEQ ID NO: 316 - protein, 23 aa, Artificial Sequence
  cyclized peptide
  MISC_FEATURE (21)..(21): is D-AA
THLETEFWGDGEPKTIRVTQPGQ

SEQ ID NO: 317 - protein, 23 aa, Artificial Sequence
  cyclized peptide
  MISC_FEATURE (6)..(6): is D-AA
  MISC_FEATURE (21)..(21): is D-AA
THLETEFWGDGEPKTIRVTQPGQ

SEQ ID NO: 318 - protein, 23 aa, Artificial Sequence
  cyclized peptide
  MISC_FEATURE (21)..(21): is D-AA
THLETDFWGDGEPKTIRVTQPGQ

SEQ ID NO: 319 - protein, 23 aa, Artificial Sequence
  cyclized peptide
  MISC_FEATURE (6)..(6): is D-AA
  MISC_FEATURE (21)..(21): is D-AA
THLETDFWGDGEPKTIRVTQPGQ

SEQ ID NO: 320 - protein, 23 aa, Artificial Sequence
  cyclized peptide
  MISC_FEATURE (6)..(6): X is Cysteic acid
  MISC_FEATURE (21)..(21): is D-AA
THLETXFWGDGEPKTIRVTQPGQ

SEQ ID NO: 321 - protein, 23 aa, Artificial Sequence
  cyclized peptide
  MISC_FEATURE (6)..(6): X is Api
  MISC_FEATURE (21)..(21): is D-AA
THLETXFWGDGEPKTIRVTQPGQ

SEQ ID NO: 322 - protein, 23 aa, Artificial Sequence
  cyclized peptide
  MISC_FEATURE (6)..(6): X is AlloThr
  MISC_FEATURE (21)..(21): is D-AA
THLETXFWGDGEPKTIRVTQPGQ

SEQ ID NO: 323 - protein, 23 aa, Artificial Sequence
  cyclized peptide
  MISC_FEATURE (5)..(5): is D-AA
  MISC_FEATURE (21)..(21): is D-AA
THLETTFWGDGEPKTIRVTQPGQ

SEQ ID NO: 324 - protein, 23 aa, Artificial Sequence
  cyclized peptide
  MISC_FEATURE (5)..(5): X is AlloThr
  MISC_FEATURE (21)..(21): is D-AA
THLEXTFWGDGEPKTIRVTQPGQ

SEQ ID NO: 325 - protein, 23 aa, Artificial Sequence
  cyclized peptide
  MISC_FEATURE (21)..(21): is D-AA
YHLETTFWGDGEPKTIRVTQPGQ

SEQ ID NO: 326 - protein, 23 aa, Artificial Sequence
  cyclized peptide
  MISC_FEATURE (1)..(1): is D-AA
  MISC_FEATURE (21)..(21): is D-AA
THLETTFWGDGEPKTIRVTQPGQ

SEQ ID NO: 327 - protein, 23 aa, Artificial Sequence
  cyclized peptide
  MISC_FEATURE (21)..(21): is D-AA
RHLETTFWGDGEPKTIRVTQPGQ

SEQ ID NO: 328 - protein, 23 aa, Artificial Sequence
  cyclized peptide
  MISC_FEATURE (1)..(1): X is Phe-4-Cl
  MISC_FEATURE (21)..(21): is D-AA
XHLETTFWGDGEPKTIRVTQPGQ

SEQ ID NO: 329 - protein, 23 aa, Artificial Sequence
  cyclized peptide
  MISC_FEATURE (1)..(1): X is Phe-3-Cl
  MISC_FEATURE (21)..(21): is D-AA
XHLETTFWGDGEPKTIRVTQPGQ

SEQ ID NO: 330 - protein, 23 aa, Artificial Sequence
  cyclized peptide
  MISC_FEATURE (1)..(1): X is Phe-2-Cl
  MISC_FEATURE (21)..(21): is D-AA
XHLETTFWGDGEPKTIRVTQPGQ

SEQ ID NO: 331 - protein, 23 aa, Artificial Sequence
  cyclized peptide
  MISC_FEATURE (1)..(1): X is Phe-4-NH2
  MISC_FEATURE (21)..(21): is D-AA
XHLETTFWGDGEPKTIRVTQPGQ

SEQ ID NO: 332 - protein, 23 aa, Artificial Sequence
  cyclized peptide
  MISC_FEATURE (21)..(21): is D-AA
KHLETTFWGDGEPKTIRVTQPGQ

SEQ ID NO: 333 - protein, 23 aa, Artificial Sequence
  cyclized peptide
  MISC_FEATURE (1)..(1): X is HomoPhe
  MISC_FEATURE (21)..(21): is D-AA
XHLETTFWGDGEPKTIRVTQPGQ

SEQ ID NO: 334 - protein, 23 aa, Artificial Sequence
  cyclized peptide
  MISC_FEATURE (1)..(1): is D-AA
  MISC_FEATURE (21)..(21): is D-AA
HHLETTFWGDGEPKTIRVTQPGQ

SEQ ID NO: 335 - protein, 23 aa, Artificial Sequence
  cyclized peptide
  MISC_FEATURE (21)..(21): is D-AA
FHLETTFWGDGEPKTIRVTQPGQ

SEQ ID NO: 336 - protein, 23 aa, Artificial Sequence
  cyclized peptide
  MISC_FEATURE (1)..(1): X is D-Phe-4-NH2
  MISC_FEATURE (21)..(21): is D-AA
XHLETTFWGDGEPKTIRVTQPGQ

SEQ ID NO: 337 - protein, 23 aa, Artificial Sequence
  cyclized peptide
  MISC_FEATURE (1)..(1): X is AlloThr
  MISC_FEATURE (21)..(21): is D-AA
XHLETTFWGDGEPKTIRVTQPGQ

SEQ ID NO: 338 - protein, 23 aa, Artificial Sequence
  cyclized peptide
  MISC_FEATURE (21)..(21): is D-AA
THVETTFWGDGEPKTIRVTQPGQ

SEQ ID NO: 339 - protein, 23 aa, Artificial Sequence
  cyclized peptide
  MISC_FEATURE (3)..(3): is D-AA
  MISC_FEATURE (21)..(21): is D-AA
THVETTFWGDGEPKTIRVTQPGQ

SEQ ID NO: 340 - protein, 23 aa, Artificial Sequence
  cyclized peptide
  MISC_FEATURE (3)..(3): X is Tle
  MISC_FEATURE (21)..(21): is D-AA
THXETTFWGDGEPKTIRVTQPGQ

SEQ ID NO: 341 - protein, 23 aa, Artificial Sequence
  cyclized peptide
  MISC_FEATURE (3)..(3): X is NVal
  MISC_FEATURE (21)..(21): is D-AA
THXETTFWGDGEPKTIRVTQPGQ

SEQ ID NO: 342 - protein, 23 aa, Artificial Sequence
  cyclized peptide
  MISC_FEATURE (3)..(3): X is NLeu
  MISC_FEATURE (21)..(21): is D-AA
THXETTFWGDGEPKTIRVTQPGQ

SEQ ID NO: 343 - protein, 23 aa, Artificial Sequence
  cyclized peptide
  MISC_FEATURE (21)..(21): is D-AA
THIETTFWGDGEPKTIRVTQPGQ

SEQ ID NO: 344 - protein, 23 aa, Artificial Sequence
  cyclized peptide
  MISC_FEATURE (3)..(3): X is HomoLeu
  MISC_FEATURE (21)..(21): is D-AA
THXETTFWGDGEPKTIRVTQPGQ

SEQ ID NO: 345 - protein, 23 aa, Artificial Sequence
  cyclized peptide
  MISC_FEATURE (3)..(3): X is HomoCha
  MISC_FEATURE (21)..(21): is D-AA
THXETTFWGDGEPKTIRVTQPGQ

SEQ ID NO: 346 - protein, 23 aa, Artificial Sequence
  cyclized peptide
  MISC_FEATURE (3)..(3): X is D-Orn
  MISC_FEATURE (21)..(21): is D-AA
THXETTFWGDGEPKTIRVTQPGQ

SEQ ID NO: 347 - protein, 23 aa, Artificial Sequence
  cyclized peptide
  MISC_FEATURE (3)..(3): X is D-HomoCha
  MISC_FEATURE (21)..(21): is D-AA
THXETTFWGDGEPKTIRVTQPGQ

SEQ ID NO: 348 - protein, 23 aa, Artificial Sequence
  cyclized peptide
  MISC_FEATURE (3)..(3): X is CycLeu
  MISC_FEATURE (21)..(21): is D-AA
THXETTFWGDGEPKTIRVTQPGQ

SEQ ID NO: 349 - protein, 23 aa, Artificial Sequence
  cyclized peptide
  MISC_FEATURE (3)..(3): X is Cha
  MISC_FEATURE (21)..(21): is D-AA
THXETTFWGDGEPKTIRVTQPGQ

SEQ ID NO: 350 - protein, 23 aa, Artificial Sequence
  cyclized peptide
  MISC_FEATURE (3)..(3): X is Aib
  MISC_FEATURE (21)..(21): is D-AA
THXETTFWGDGEPKTIRVTQPGQ

SEQ ID NO: 351 - protein, 23 aa, Artificial Sequence
  cyclized peptide
  MISC_FEATURE (3)..(3): X is Abu
  MISC_FEATURE (21)..(21): is D-AA
THXETTFWGDGEPKTIRVTQPGQ

SEQ ID NO: 352 - protein, 23 aa, Artificial Sequence
  cyclized peptide
  MISC_FEATURE (21)..(21): is D-AA
TYLETTFWGDGEPKTIRVTQPGQ

SEQ ID NO: 353 - protein, 23 aa, Artificial Sequence
  cyclized peptide
  MISC_FEATURE (2)..(2): X is Thi
  MISC_FEATURE (21)..(21): is D-AA
TXLETTFWGDGEPKTIRVTQPGQ

SEQ ID NO: 354 - protein, 23 aa, Artificial Sequence
  cyclized peptide
  MISC_FEATURE (21)..(21): is D-AA
TRLETTFWGDGEPKTIRVTQPGQ

SEQ ID NO: 355 - protein, 23 aa, Artificial Sequence
  cyclized peptide
  MISC_FEATURE (2)..(2): X is Orn
  MISC_FEATURE (21)..(21): is D-AA
TXLETTFWGDGEPKTIRVTQPGQ

SEQ ID NO: 356 - protein, 23 aa, Artificial Sequence
  cyclized peptide
  MISC_FEATURE (2)..(2): X is Phe-4-NH2
  MISC_FEATURE (21)..(21): is D-AA
TXLETTFWGDGEPKTIRVTQPGQ

SEQ ID NO: 357 - protein, 23 aa, Artificial Sequence
  cyclized peptide
  MISC_FEATURE (21)..(21): is D-AA
TKLETTFWGDGEPKTIRVTQPGQ

SEQ ID NO: 358 - protein, 23 aa, Artificial Sequence
  cyclized peptide
  MISC_FEATURE (21)..(21): is D-AA
TKLETTFWGDGEPKTIRVTQPGQ

SEQ ID NO: 359 - protein, 23 aa, Artificial Sequence
  cyclized peptide
  MISC_FEATURE (2)..(2): X is HomoLys
  MISC_FEATURE (21)..(21): is D-AA
TXLETTFWGDGEPKTIRVTQPGQ

SEQ ID NO: 360 - protein, 23 aa, Artificial Sequence
  cyclized peptide
  MISC_FEATURE (2)..(2): X is HomoCit
  MISC_FEATURE (21)..(21): is D-AA
TXLETTFWGDGEPKTIRVTQPGQ

SEQ ID NO: 361 - protein, 23 aa, Artificial Sequence
  cyclized peptide
  MISC_FEATURE (2)..(2): X is HomoArg
  MISC_FEATURE (21)..(21): is D-AA
TXLETTFWGDGEPKTIRVTQPGQ

SEQ ID NO: 362 - protein, 23 aa, Artificial Sequence
  cyclized peptide
  MISC_FEATURE (2)..(2): X is His-3Me
  MISC_FEATURE (21)..(21): is D-AA
TXLETTFWGDGEPKTIRVTQPGQ

SEQ ID NO: 363 - protein, 23 aa, Artificial Sequence
  cyclized peptide
  MISC_FEATURE (2)..(2): X is His-1Me
  MISC_FEATURE (21)..(21): is D-AA
TXLETTFWGDGEPKTIRVTQPGQ

SEQ ID NO: 364 - protein, 23 aa, Artificial Sequence
  cyclized peptide
  MISC_FEATURE (2)..(2): X is FurAla
  MISC_FEATURE (21)..(21): is D-AA
TXLETTFWGDGEPKTIRVTQPGQ

SEQ ID NO: 365 - protein, 23 aa, Artificial Sequence
  cyclized peptide
  MISC_FEATURE (2)..(2): X is PyA-4
  MISC_FEATURE (21)..(21): is D-AA
TXLETTFWGDGEPKTIRVTQPGQ

SEQ ID NO: 366 - protein, 23 aa, Artificial Sequence
  cyclized peptide
  MISC_FEATURE (2)..(2): X is Phe-4-N3
  MISC_FEATURE (21)..(21): is D-AA
TXLETTFWGDGEPKTIRVTQPGQ

SEQ ID NO: 367 - protein, 23 aa, Artificial Sequence
  cyclized peptide
  MISC_FEATURE (11)..(11): X is R(Me2)
  MISC_FEATURE (21)..(21): is D-AA
THLETTFWGDXEPKTIRVTQPGQ

SEQ ID NO: 368 - protein, 23 aa, Artificial Sequence
  cyclized peptide
  MISC_FEATURE (2)..(2): X is Phe-4-CH2NH2
  MISC_FEATURE (21)..(21): is D-AA
TXLETTFWGDGEPKTIRVTQPGQ

SEQ ID NO: 369 - protein, 23 aa, Artificial Sequence
  cyclized peptide
  MISC_FEATURE (11)..(11): X is R(Me)
  MISC_FEATURE (21)..(21): is D-AA
THLETTFWGDXEPKTIRVTQPGQ

SEQ ID NO: 370 - protein, 23 aa, Artificial Sequence
  cyclized peptide
  MISC_FEATURE (21)..(21): is D-AA
THLETTFWGDREPKTIRVTQPGQ

SEQ ID NO: 371 - protein, 23 aa, Artificial Sequence
  cyclized peptide
  MISC_FEATURE (21)..(21): is D-AA
THLETTFWGDPEPKTIRVTQPGQ

SEQ ID NO: 372 - protein, 23 aa, Artificial Sequence
  cyclized peptide
  MISC_FEATURE (11)..(11): is D-AA
  MISC_FEATURE (21)..(21): is D-AA
THLETTFWGDPEPKTIRVTQPGQ

SEQ ID NO: 373 - protein, 23 aa, Artificial Sequence
  cyclized peptide
  MISC_FEATURE (21)..(21): is D-AA
THLETTFWGDAEPKTIRVTQPGQ

SEQ ID NO: 374 - protein, 23 aa, Artificial Sequence
  cyclized peptide
  MISC_FEATURE (11)..(11): is D-AA
  MISC_FEATURE (21)..(21): is D-AA
THLETTFWGDAEPKTIRVTQPGQ

SEQ ID NO: 375 - protein, 23 aa, Artificial Sequence
  cyclized peptide
  MISC_FEATURE (9)..(9): X is HomoGln
  MISC_FEATURE (21)..(21): is D-AA
THLETTFWXDGEPKTIRVTQPGQ

SEQ ID NO: 376 - protein, 23 aa, Artificial Sequence
  cyclized peptide
  MISC_FEATURE (9)..(9): X is HomoCit
  MISC_FEATURE (21)..(21): X is HomoCit
THLETTFWXDGEPKTIRVTQPGQ

SEQ ID NO: 377 - protein, 23 aa, Artificial Sequence
  cyclized peptide
  MISC_FEATURE (9)..(9): X is D-Cit
  MISC_FEATURE (21)..(21): is D-AA
THLETTFWXDGEPKTIRVTQPGQ

SEQ ID NO: 378 - protein, 23 aa, Artificial Sequence
  cyclized peptide
  MISC_FEATURE (9)..(9): X is Cit
  MISC_FEATURE (21)..(21): is D-AA
THLETTFWXDGEPKTIRVTQPGQ

SEQ ID NO: 379 - protein, 23 aa, Artificial Sequence
  cyclized peptide
  MISC_FEATURE (7)..(7): X is StyAla
  MISC_FEATURE (21)..(21): is D-AA
THLETTXWGDGEPKTIRVTQPGQ

SEQ ID NO: 380 - protein, 23 aa, Artificial Sequence
  cyclized peptide
  MISC_FEATURE (7)..(7): X is Phg
  MISC_FEATURE (21)..(21): is D-AA
THLETTXWGDGEPKTIRVTQPGQ

SEQ ID NO: 381 - protein, 23 aa, Artificial Sequence
  cyclized peptide
  MISC_FEATURE (7)..(7): X is Phe-4-F
  MISC_FEATURE (21)..(21): is D-AA
THLETTXWGDGEPKTIRVTQPGQ

SEQ ID NO: 382 - protein, 23 aa, Artificial Sequence
  cyclized peptide
  MISC_FEATURE (7)..(7): X is Phe-4-Cl
THLETTXWGDGEPKTIRVTQPGQ

SEQ ID NO: 383 - protein, 23 aa, Artificial Sequence
  cyclized peptide
  MISC_FEATURE (7)..(7): X is Phe-4-Br
  MISC_FEATURE (21)..(21): is D-AA
THLETTXWGDGEPKTIRVTQPGQ

SEQ ID NO: 384 - protein, 23 aa, Artificial Sequence
  cyclized peptide
  MISC_FEATURE (7)..(7): X is Phe-3-F
  MISC_FEATURE (21)..(21): is D-AA
THLETTXWGDGEPKTIRVTQPGQ

SEQ ID NO: 385 - protein, 23 aa, Artificial Sequence
  cyclized peptide
  MISC_FEATURE (7)..(7): X is Phe-3-Cl
  MISC_FEATURE (21)..(21): is D-AA
THLETTXWGDGEPKTIRVTQPGQ

SEQ ID NO: 386 - protein, 23 aa, Artificial Sequence
  cyclized peptide
  MISC_FEATURE (7)..(7): X is Phe-3-Br
  MISC_FEATURE (21)..(21): is D-AA
THLETTXWGDGEPKTIRVTQPGQ

SEQ ID NO: 387 - protein, 23 aa, Artificial Sequence
  cyclized peptide
  MISC_FEATURE (7)..(7): X is Phe-2-F
  MISC_FEATURE (21)..(21): is D-AA
THLETTXWGDGEPKTIRVTQPGQ

SEQ ID NO: 388 - protein, 23 aa, Artificial Sequence
  cyclized peptide
  MISC_FEATURE (7)..(7): X is Phe-2-Cl
  MISC_FEATURE (21)..(21): is D-AA
THLETTXWGDGEPKTIRVTQPGQ

SEQ ID NO: 389 - protein, 23 aa, Artificial Sequence
  cyclized peptide
  MISC_FEATURE (7)..(7): X is Phe-2-Br
  MISC_FEATURE (21)..(21): is D-AA
THLETTXWGDGEPKTIRVTQPGQ

SEQ ID NO: 390 - protein, 23 aa, Artificial Sequence
  cyclized peptide
  MISC_FEATURE (7)..(7): X is Nal2
  MISC_FEATURE (21)..(21): is D-AA
THLETTXWGDGEPKTIRVTQPGQ

SEQ ID NO: 391 - protein, 23 aa, Artificial Sequence
  cyclized peptide
  MISC_FEATURE (7)..(7): X is Nal1
  MISC_FEATURE (21)..(21): is D-AA
THLETTXWGDGEPKTIRVTQPGQ

SEQ ID NO: 392 - protein, 23 aa, Artificial Sequence
  cyclized peptide
  MISC_FEATURE (7)..(7): X is HomoCha
  MISC_FEATURE (21)..(21): is D-AA
THLETTXWGDGEPKTIRVTQPGQ

SEQ ID NO: 393 - protein, 23 aa, Artificial Sequence
  cyclized peptide
  MISC_FEATURE (7)..(7): X is HomoPhe
  MISC_FEATURE (21)..(21): is D-AA
THLETTXWGDGEPKTIRVTQPGQ

SEQ ID NO: 394 - protein, 23 aa, Artificial Sequence
  cyclized peptide
  MISC_FEATURE (7)..(7): X is Phe-4-(tBu)
  MISC_FEATURE (21)..(21): is D-AA
THLETTXWGDGEPKTIRVTQPGQ

SEQ ID NO: 395 - protein, 23 aa, Artificial Sequence
  cyclized peptide
  MISC_FEATURE (7)..(7): is D-AA
  MISC_FEATURE (21)..(21): is D-AA
THLETTFWGDGEPKTIRVTQPGQ

SEQ ID NO: 396 - protein, 23 aa, Artificial Sequence
  cyclized peptide
  MISC_FEATURE (7)..(7): X is D-HomoCha
  MISC_FEATURE (21)..(21): is D-AA
THLETTXWGDGEPKTIRVTQPGQ

SEQ ID NO: 397 - protein, 23 aa, Artificial Sequence
  cyclized peptide
  MISC_FEATURE (7)..(7): X is D-Cha
  MISC_FEATURE (21)..(21): is D-AA
THLETTXWGDGEPKTIRVTQPGQ

SEQ ID NO: 398 - protein, 23 aa, Artificial Sequence
  cyclized peptide
  MISC_FEATURE (7)..(7): X is Cha
  MISC_FEATURE (21)..(21): is D-AA
THLETTXWGDGEPKTIRVTQPGQ

SEQ ID NO: 399 - protein, 23 aa, Artificial Sequence
  cyclized peptide
  MISC_FEATURE (7)..(7): X is BIP
  MISC_FEATURE (21)..(21): is D-AA
THLETTXWGDGEPKTIRVTQPGQ

SEQ ID NO: 400 - protein, 23 aa, Artificial Sequence
  cyclized peptide
  MISC_FEATURE (7)..(7): X is Phe-4-Me
  MISC_FEATURE (21)..(21): is D-AA
THLETTXWGDGEPKTIRVTQPGQ

SEQ ID NO: 401 - protein, 23 aa, Artificial Sequence
  cyclized peptide
  MISC_FEATURE (7)..(7): X is Phe-3-Me
  MISC_FEATURE (21)..(21): is D-AA
THLETTXWGDGEPKTIRVTQPGQ

SEQ ID NO: 402 - protein, 23 aa, Artificial Sequence
  cyclized peptide
  MISC_FEATURE (7)..(7): X is Phe-2-Me
  MISC_FEATURE (21)..(21): is D-AA
THLETTXWGDGEPKTIRVTQPGQ

SEQ ID NO: 403 - protein, 23 aa, Artificial Sequence
  cyclized peptide
  MISC_FEATURE (21)..(21): is D-AA
THLRTTFWGDGEPKTIRVTQPGQ

SEQ ID NO: 404 - protein, 23 aa, Artificial Sequence
  cyclized peptide
  MISC_FEATURE (21)..(21): is D-AA
THLQTTFWGDGEPKTIRVTQPGQ

SEQ ID NO: 405 - protein, 23 aa, Artificial Sequence
  cyclized peptide
  MISC_FEATURE (4)..(4): X is Orn
  MISC_FEATURE (21)..(21): is D-AA
THLXTTFWGDGEPKTIRVTQPGQ

SEQ ID NO: 406 - protein, 23 aa, Artificial Sequence
  cyclized peptide
  MISC_FEATURE (4)..(4): X is HomoSer
  MISC_FEATURE (21)..(21): is D-AA
THLXTTFWGDGEPKTIRVTQPGQ

SEQ ID NO: 407 - protein, 23 aa, Artificial Sequence
  cyclized peptide
  MISC_FEATURE (4)..(4): X is HomoGln
  MISC_FEATURE (21)..(21): is D-AA
THLXTTFWGDGEPKTIRVTQPGQ

SEQ ID NO: 408 - protein, 23 aa, Artificial Sequence
  cyclized peptide
  MISC_FEATURE (4)..(4): X is Glu(OAll)
  MISC_FEATURE (21)..(21): is D-AA
THLXTTFWGDGEPKTIRVTQPGQ

SEQ ID NO: 409 - protein, 23 aa, Artificial Sequence
  cyclized peptide
  MISC_FEATURE (4)..(4): is D-AA
  MISC_FEATURE (21)..(21): is D-AA
THLETTFWGDGEPKTIRVTQPGQ

SEQ ID NO: 410 - protein, 23 aa, Artificial Sequence
  cyclized peptide
  MISC_FEATURE (21)..(21): is D-AA
THLDTTFWGDGEPKTIRVTQPGQ

SEQ ID NO: 411 - protein, 23 aa, Artificial Sequence
  cyclized peptide
  MISC_FEATURE (4)..(4): is D-AA
  MISC_FEATURE (21)..(21): is D-AA
THLDTTFWGDGEPKTIRVTQPGQ

SEQ ID NO: 412 - protein, 23 aa, Artificial Sequence
  cyclized peptide
  MISC_FEATURE (4)..(4): X is cysteic acid
  MISC_FEATURE (20)..(20): is D-AA
THLXTTFWGDGEPKTIRVTQPGQ

SEQ ID NO: 413 - protein, 23 aa, Artificial Sequence
  cyclized peptide
  MISC_FEATURE (21)..(21): is D-AA
THLETTFWGEGEPKTIRVTQPGQ

SEQ ID NO: 414 - protein, 23 aa, Artificial Sequence
  cyclized peptide
  MISC_FEATURE (4)..(4): is D-AA
  MISC_FEATURE (10)..(10): is D-AA
  MISC_FEATURE (21)..(21): is D-proline
THLDTTFWGEGEPKTIRVTQPGQ

SEQ ID NO: 415 - protein, 23 aa, Artificial Sequence
  cyclized peptide
  MISC_FEATURE (4)..(4): is D-AA
  MISC_FEATURE (10)..(10): is D-AA
  MISC_FEATURE (21)..(21): is D-proline
THLDTTFWGDGEPKTIRVTQPGQ

SEQ ID NO: 416 - protein, 20 aa, Artificial Sequence
  synthetic peptide
THLETTFWGDGEPKTIRVTQ

SEQ ID NO: 417 - protein, 19 aa, Artificial Sequence
  synthetic peptide
THLETTFGDGEPKTIRVTQ

SEQ ID NO: 418 - protein, 22 aa, Artificial Sequence
  synthetic peptide
THMELETTFWGDGEPKTIRVTQ

SEQ ID NO: 419 - protein, 21 aa, Artificial Sequence
  synthetic peptide
THMELETTFGDGEPKTIRVTQ

SEQ ID NO: 420 - protein, 23 aa, Artificial Sequence
  cyclized peptide
  MISC_FEATURE (21)..(21): is D-proline
THLETTFWGDGEPKTIRVTQPGE

SEQ ID NO: 421 - protein, 22 aa, Artificial Sequence
  cyclized peptide
  MISC_FEATURE (20)..(20): is D-proline
THLETTFGDGEPKTIRVTQPGE

SEQ ID NO: 422 - protein, 23 aa, Artificial Sequence
  cyclized peptide
  MISC_FEATURE (2)..(2): X is H-3Me
  MISC_FEATURE (21)..(21): is D-proline
TXLETTFWGDGEPKTIRVTQPGE

SEQ ID NO: 423 - protein, 22 aa, Artificial Sequence
  cyclized peptide
  MISC_FEATURE (2)..(2): X is H-3Me
  MISC_FEATURE (20)..(20): is D-proline
TXLETTFGDGEPKTIRVTQPGE

SEQ ID NO: 424 - protein, 23 aa, Artificial Sequence
  cyclized peptide
  MISC_FEATURE (14)..(14): X is K-CA
  MISC_FEATURE (21)..(21): is D-proline
THLETTFTGDGTPXTIRVTQPGQ

SEQ ID NO: 425 - protein, 23 aa, Artificial Sequence
  cyclized peptide
  MISC_FEATURE (22)..(22): is D-proline
CTHLETTFTGDGTPKTIRVTQPG

SEQ ID NO: 426 - protein, 20 aa, Artificial Sequence
  synthetic peptide
THLETTFTGDGTPKTIRVTQ

SEQ ID NO: 427 - protein, 23 aa, Artificial Sequence
  cyclized peptide
  MISC_FEATURE (1)..(1): X is Ac-T
  MISC_FEATURE (21)..(21): is D-proline
XHLETTFTGDGTPKTIRVTQPGC

SEQ ID NO: 428 - protein, 23 aa, Artificial Sequence
  cyclized peptide
  MISC_FEATURE (21)..(21): is D-proline
THLETTFTGDGTPKTIRVTQPGE

SEQ ID NO: 429 - protein, 23 aa, Artificial Sequence
  cyclized peptide
  MISC_FEATURE (21)..(21): is D-proline
THLETTFTGDGTPKTIRVTQPGD

SEQ ID NO: 430 - protein, 23 aa, Artificial Sequence
  cyclized peptide
  MISC_FEATURE (21)..(21): is D-proline
THLETTFTGDGTPKTIRVTQPGE

SEQ ID NO: 431 - protein, 5 aa, Artificial Sequence
  synthetic peptide
LETTF

SEQ ID NO: 432 - protein, 9 aa, Artificial Sequence
  synthetic peptide
KLSSIESDV

SEQ ID NO: 433 - protein, 10 aa, Artificial Sequence
  markush structure
  MISC_FEATURE (4)..(4): X is T, S, D or E
  MISC_FEATURE (5)..(5): X is F, F-2-Br, F-2-Cl or F-3-F
  MISC_FEATURE (6)..(6): X is W, Nal or absent
  MISC_FEATURE (8)..(8): X is D or N-Me-D
  MISC_FEATURE (9)..(9): X is G, A or P
  MISC_FEATURE (10)..(10): X is E or D
LETXXXGXXX

SEQ ID NO: 434 - DNA, 17 bp, Artificial Sequence
  primer
  misc_feature (1)..(17): K165A forward
ctgatcgcgggcccgaa

SEQ ID NO: 435 - DNA, 24 bp, Artificial Sequence
  primer
  misc_feature (1)..(24): K165A reverse
tttgatttccatcactttttccgc

SEQ ID NO: 436 - DNA, 16 bp, Artificial Sequence
  primer
  misc_feature (1)..(16): K168A forward
cccggcgggcctgggc

SEQ ID NO: 437 - DNA, 21 bp, Artificial Sequence
  primer
  misc_feature (1)..(21): K168A reverse
cctttgatcagtttgatttcc

SEQ ID NO: 438 - DNA, 22 bp, Artificial Sequence
  primer
  misc_feature (1)..(22): K193A forward
gtgaccgcgattattgaaggcg

SEQ ID NO: 439 - DNA, 21 bp, Artificial Sequence
  primer
  misc_feature (1)..(21): K193A reverse
ataaatgctgttatcgcccgg

SEQ ID NO: 440 - DNA, 20 bp, Artificial Sequence
  primer
  misc_feature (1)..(20): H225A forward
gatgtgatggcggaagatgc

SEQ ID NO: 441 - DNA, 19 bp, Artificial Sequence
  primer
  misc_feature (1)..(19): H225A reverse
ttccaggcccacgctgttc

SEQ ID NO: 442 - DNA, 20 bp, Artificial Sequence
  primer
  misc_feature (1)..(20): K233A forward
gcgctggcgaacacctatga

SEQ ID NO: 443 - DNA, 20 bp, Artificial Sequence
  primer
  misc_feature (1)..(20): K233A reverse
cgccaccgcatcttcatgca

SEQ ID NO: 444 - DNA, 20 bp, Artificial Sequence
  primer
  misc_feature (1)..(20): F172I forward
ctgggcattagcattgcggg

SEQ ID NO: 445 - DNA, 21 bp, Artificial Sequence
  primer
  misc_feature (1)..(21): F172I reverse
gcctttcgggcctttgatcag

SEQ ID NO: 446 - DNA, 20 bp, Artificial Sequence
  primer
  misc_feature (1)..(20): S173A forward
gctttgcgattgcgggcggt

SEQ ID NO: 447 - DNA, 22 bp, Artificial Sequence
  primer
  misc_feature (1)..(22): S173A reverse
ccaggcctttcgggcctttgat

SEQ ID NO: 448 - DNA, 33 bp, Artificial Sequence
  primer
  misc_feature (1)..(33): T192A forward
catttatgtggcgaaaattattgaaggcggtgc

SEQ ID NO: 449 - DNA, 27 bp, Artificial Sequence
  primer
  misc_feature (1)..(27): T192A reverse
ctgttatcgcccggaatatgctggttg

SEQ ID NO: 450 - DNA, 21 bp, Artificial Sequence
  primer
  misc_feature (1)..(21): V229A forward
catgaagatgcggcagcggcg

SEQ ID NO: 451 - DNA, 23 bp, Artificial Sequence
  primer
  misc_feature (1)..(23): V229A reverse
catcacatcttccaggcccacgc

SEQ ID NO: 452 - DNA, 22 bp, Artificial Sequence
  primer
  misc_feature (1)..(22): N180A forward
ggtgtgggcgcgcagcatattc

SEQ ID NO: 453 - DNA, 23 bp, Artificial Sequence
  primer
  misc_feature (1)..(23): N180A reverse
gcccgcaatgctaaagcccaggc

SEQ ID NO: 454 - DNA, 21 bp, Artificial Sequence
  primer
  misc_feature (1)..(21): E226A forward
gtgatgcatgcggatgcggtg

SEQ ID NO: 455 - DNA, 22 bp, Artificial Sequence
  primer
  misc_feature (1)..(22): E226A reverse
atcttccaggcccacgctgttc

SEQ ID NO: 456 - DNA, 20 bp, Artificial Sequence
  primer
  misc_feature (1)..(20): F172A forward
ctgggcgcgagcattgcggg

SEQ ID NO: 457 - DNA, 21 bp, Artificial Sequence
  primer
  misc_feature (1)..(21): F172A reverse
gcctttcgggcctttgatcag

(19) United States
(12) Patent Application Publication
(10) Pub. No.: US 2023/0220021 A1
(43) Pub. Date: Jul. 13, 2023

(54) CYCLIC PEPTIDE INHIBITORS OF PSD-95 AND USES THEREOF

(71) Applicant: University of Copenhagen, Copenhagen K (DK)

(72) Inventors: Javier Rodriguez Balboa, Copenhagen (DK); Kristian Strømgaard, Copenhagen (DK); Søren Østergaard, Copenhagen (DK)

(21) Appl. No.: 18/000,946

(22) PCT Filed: Jun. 11, 2021
(86) PCT No.: PCT/EP2021/065734

§ 371 Date: Dec. 7, 2022

(30) Foreign Application Priority Data
Jun. 11, 2020 (EP) 2017519.2

(51) Int. Cl. C07K 14/47; C12N 9/02

(52) U.S. Cl. CPC C07K 14/47; C12N 9/0075; C12Y 114/13039

(57) ABSTRACT

The present invention relates novel cyclic peptides which can act as inhibitors of protein-protein interactions, specifically by inhibiting the PDZ2 domain of PSD-95, as well as their use in treatment of excitotoxic-related diseases and neuropathic pain.

### TECHNICAL FIELD

The present invention relates novel cyclic peptides which can act as inhibitors of protein-protein interactions, specifically by inhibiting the PDZ1 and/or PDZ2 domain of PSD-95, as well as their use in treatment of excitotoxic-related diseases and neuropathic pain.

### BACKGROUND

Thirteen million people around the globe suffer from stroke annually, being the second major cause of death and disability. The ternary complex between the N-methyl-D-aspartate receptor (NMDAR), postsynaptic density protein-95 (PSD-95) and neuronal nitric oxide synthase (nNOS) plays an important role in the excitotoxicity mechanism of cell death (FIG. 1).

PSD-95 is a protein encoded in humans by the DLG4 (disks large homolog 4) gene. PSD-95 is a member of the membrane-associated guanylate kinase (MAGUK) family and is together with PSD-93 recruited into the same NMDA receptor and potassium channel clusters.

PSD-95 is almost exclusively located in the postsynaptic density of neurons, and is involved in anchoring synaptic proteins. Its direct and indirect binding partners include neuroligin, nNOS, NMDA receptors, AMPA receptors, and potassium channels.

PSD-95 includes three PDZ domains, an SH3 domain, and a guanylate kinase-like (GK) domain connected by linker regions. The PDZ1 and PDZ2 domains of PSD-95 interact with several proteins including the simultaneous binding of the NMDA receptor-type of ionotropic glutamate receptors and the nitric oxide (NO) producing enzyme nNOS.

NMDA receptors are the principal mediators of excitotoxicity, i.e. glutamate-mediated neurotoxicity, which is implicated in neurodegenerative diseases and acute brain injuries. Although antagonists of the NMDA receptor efficiently reduce excitotoxicity by preventing glutamate-mediated ionflux, they also prevent physiologically important processes. Thus, NMDA receptor antagonists have failed in clinical trials for e.g. stroke due to low tolerance and lack of efficacy. Instead, specific inhibition of excitotoxicity can be obtained by perturbing the intracellular nNOS/PSD-95/NMDA receptor complex using PSD-95 inhibitors.

Several pointers now suggests that some PDZ domains can also recognize internal binding motifs, a less explored PDZ binding mode. An example of such interaction is found between the PDZ2 domain of PSD-95 and the neuronal nitric oxide synthase (nNOS). At a molecular level, this interaction involves a 30-residue stretch within nNOS that adopts an extended β-hairpin fold without a free C-terminus.

Numerous attempts have been made to design nNOS or NMDA receptor inhibitors to induce neuroprotection after ischemic stroke. Still, side-effects, reduced efficacy, lack of selectivity and low blood-brain barrier permeability led to poor success in this regard. Consequently, there is still a need in the field for an efficient inhibitor of such protein-protein interactions.

### SUMMARY

In order to address the stated problem of providing inhibitors of protein-protein interactions by specifically targeting the PDZ domains of PSD-95, the present disclosure describes a novel class of cyclic peptides capable of non-canonical binding to the PDZ1 and/or PDZ2 domain of PSD-95, thereby inhibiting its protein-protein interaction with nNOS for treatment of ischemic stroke and neuropathic pain, methods of manufacture and use of said peptides.

In one aspect, the present invention relates to a polypeptide comprising the amino acid sequence of TX₁LETX₂X₃X₄GX₅X₆X₇PX₈TIRVX₉Q (SEQ ID NO: 1), wherein
X₁ is H, H-3Me or PyA-4;
X₂ is T, S, D or E;
X₃ is F, F-2-Br, F-2-Cl or F-3-F;
X₄ is W, NaI, or absent;
X₅ is D or N-Me-D;
X₆ is G, A or P;
X₇ is E or D;
X₈ is K or N-Me-K; and
X₉ is T or N-Me-T;

or a pharmaceutically acceptable salt thereof.

In one embodiment, the polypeptide is a cyclic polypeptide.

In one aspect, the present invention relates to a polypeptide, such as a cyclic polypeptide, as defined herein for use as a medicament.

In one aspect, the present invention relates to a polypeptide, such as a cyclic polypeptide, as defined herein for use in prevention and/or treatment of an excitotoxic-related disease in a subject.

In one aspect, the present invention relates to a polypeptide, such as a cyclic polypeptide, as defined herein for use in prevention and/or treatment of neuropathic pain in a subject.

In one aspect, the present invention concerns a method for manufacturing the polypeptide as defined herein, said method comprising the steps of:
a) preparing a peptide using Fmoc/tBu-based solid-phase peptide synthesis (SPPS), and
b) cyclization of said peptide via native chemical ligation (NCL).

This project has received funding from the European Union's Horizon 2020 research and innovation programme under the Marie Skłlodowska-Curie grant agreement No 675341.

### DESCRIPTION OF THE DRAWINGS

FIG. 1: Schematic representation of excitotoxicity during ischemic stroke showing the abnormal influx of Ca⁺² inside the post-synapse through the NMDA receptor, which is binding to PSD-95-PDZ1. Then nNOS, which is binding to PSD-95-PDZ2 through an internal hairpin motif, produces an excess of NO, causing excitotoxicity.

FIG. 2: ITC raw heat signature (upper panel) and binding isotherm (lower panel) for the cyclic nNOS β-hairpin peptide and the nNOS linear peptide with PSD-95-PDZ2 at 25° C. Data was collected in triplicates and the association constant values (Kₐ) values were converted to K_{d}. Values are presented as the mean of K_{d} values±SEM.

FIG. 3: Kᵢ values of different nNOS β-hairpin cyclic scaffolds measured against recombinantly expressed PSD-95-PDZ2 and the cyclic nNOS TAM RA probe in FP competition experiments. The different ring closures are represented on the right-side. Data was collected in triplicates and is presented as mean of Kᵢ values±SEM.

FIG. 4: Deep mutational scan heat map of the nNOS β-hairpin motif screened in SPOT arrays with TAMRA labelled PSD-95-PDZ2. Fluorescence values have been normalized to the cyclic nNOS β-hairpin peptide WT value. The residues have been classified according to the side chain properties and the native residues are show on top inside a scheme of the nNOS β-hairpin. WT residues are indicated with a fill pattern cell.

FIG. 5: A) Alanine scan correlation between the normalized FP inhibition constants and the normalized fluorescence values of the SPOT peptide array. B) Mutational analysis correlation between the normalized FP inhibition constants and the normalized fluorescence values of the SPOT peptide array.

FIG. 6: A) K values of the Ala scan of the cyclic nNOS β-hairpin peptide measured against recombinantly expressed PSD-95-PDZ2 and the cyclic nNOS TAMRA probe in FP competition experiments. Data was collected in triplicates and is presented as mean of Kᵢ values±SEM. B) Model of the cyclic nNOS β-hairpin/PSD-95-PDZ2 domain with relevant H-bonds and residues highlighted. C) H-bond interactions of E108 with T192 and S173 of PSD-95-PDZ2. D) H-bond side chain interactions between T109 and T119 of the cyclic nNOS β-hairpin peptide with H225 of PSD-95-PDZ2. E) F111 side chain projection inside the hydrophobic pocket of the PSD-95-PDZ2 domain. Not binding (N.B.).

FIG. 7: A) K values of the N-Me scan of the cyclic nNOS β-hairpin peptide measured against recombinantly expressed PSD-95-PDZ2 and the cyclic nNOS TAMRA probe in FP competition experiments. Data was collected in triplicates and is presented as mean of Kᵢ values±SEM. B) Backbone H-bonds and relevant residues highlighted in the model structure.

FIG. 8: A) Sequences of the cyclic nNOS β-finger and the GluN2B. Relevant positions are indicated on top. B) Fold change of the cyclic nNOS TAM RA probe (PSD-95-PDZ2 WT K_{d}=1.0±0.1 μM) measured against recombinantly expressed PSD-95-PDZ2 Ala mutants in FP saturation experiments. C) Fold change of the C-terminal GluN2B TAM RA probe (PSD-95-PDZ2 WT K_{d}=4.0±0.1 μM) measured against recombinantly expressed PSD-95-PDZ2 Ala mutants in FP saturation experiments. Relevant residues are highlighted in the model structure.

FIG. 9: Volcano plot comparing the enrichment of isolated proteins from homogenized neuronal tissue (adult mouse) in the membranal fraction. Proteins enriched by Dynabeads™ M-270 labelled with cyclic nNOS β-hairpin peptide are shown on the left side of the plot while proteins enriched by Dynabeads™ 270 labelled with GluN2B C-terminal peptide are shown on the right-side of the plot.

FIG. 10: Volcano plot comparing the enrichment of isolated proteins from homogenized neuronal tissue (adult mouse) in the cytosolic fraction. Proteins enriched by Dynabeads™ M-270 labelled with cyclic nNOS β-hairpin peptide are shown on the left side of the plot while proteins enriched by Dynabeads™ M-270 labelled with GluN2B C-terminal peptide are shown on the right-side of the plot.

FIG. 11: ITC raw heat signature (upper panel) and binding isotherm (lower panel) for A) the wild-type cyclic nNOS β-hairpin peptide, B) cyclic nNOS β-finger mimic peptide with mutation T112W and T116E, C) cyclic nNOS β-finger mimic peptide with mutation ΔT112 and T116E, D) cyclic nNOS β-finger mimic peptide with mutation H106H(3-Me), T112W and T116E, E) cyclic nNOS β-finger mimic peptide with mutation H106H(3-Me), ΔT112 and T116E. The peptide structure is represented with a ribbon diagram above every titration curve, with substitution side chains represented with stick diagrams.

FIG. 12: Half-life of cyclic nNOS β-hairpin peptide analogues as determined in a plasmin stability assay. Data presented as mean n=3.

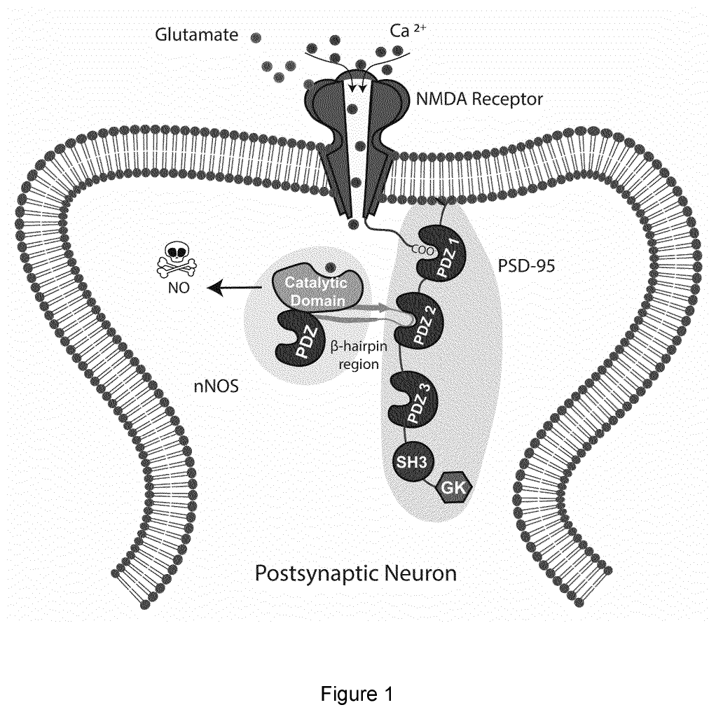

### DETAILED DESCRIPTION

The invention is as defined in the claims.

Definitions

Proteinogenic “amino acids” (AA) are named herein using either their 1-letter or 3-letter code according to the recommendations from IUPAC, see for example http://www.chem.qmul.ac.uk/iupac/AminoAcid/. Capital letter abbreviations indicate L-amino acids, whereas lower case letter abbreviations indicate D-amino acids. Unless otherwise stated, the amino acid is α-amino acid, i.e. an amido acid having both the amine and the carboxylic acid groups attached to the a-carbon atom.

The term “cell penetrating peptide” (CPP) refers to a peptide characterised by the ability to cross the plasma membrane of mammalian cells, and thereby ability to facilitate the intracellular delivery of cargo molecules, such as peptides, proteins or oligonucleotides to which it is linked.

The term “detectable moiety” refers to a moiety, which can be detected by analytical means. A detectable moiety may be selected from the group consisting of fluorophores, radiocontrasts, MRI contrast agents and radioisotopes.

The term “effective amount”, as used herein, refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects.

The term “K_{d}” refers to a dissociation constant and is a measure of the affinity of a molecule for another molecule. The lower the K_{d}, the higher the affinity of a peptide for its binding site.

The term “non-proteinogenic amino acids”, also referred to as non-canonical non-coded, non-standard, non-cognate, unnatural or non-natural amino acids, are amino acids, as used herein which are not encoded by the genetic code. A non-exhaustive list of non-proteinogenic amino acids include α-amino-n-butyric acid, norvaline, norleucine, isoleucine, alloisoleucine, tert-leucine, α-amino-n-heptanoic acid, pipecolic acid, α,β-diaminopropionic acid, α,γ-diaminobutyric acid, ornithine, allothreonine, homocysteine, homoserine, β-alanine, β-amino-n-butyric acid, β-aminoisobutyric acid, γ-aminobutyric acid, α-aminoisobutyric acid, isovaline, sarcosine, N-ethyl glycine, N-propyl glycine, N-isopropyl glycine, N-methyl alanine, N-ethyl alanine, N-methyl β-alanine, N-ethyl β-alanine, isoserine and α-hydroxy-γ-aminobutyric acid.

The term “polypeptide”, “peptide” or “protein” refers to a polymer of amino acid residues preferably joined exclusively by peptide bonds, whether produced naturally or synthetically. The term “polypeptide” as used herein covers proteins, peptides and polypeptides, wherein said proteins, peptides or polypeptides may or may not have been post-translationally modified. A peptide is usually shorter in length than a protein, and single-chained.

The term “PDZ” refers to Postsynaptic density protein-95 (PSD-95), Drosophila homologue discs large tumor suppressor (DIgA), Zonula occludens-1 protein (zo-1).

The term “PSD-95” refers to the protein PSD-95 (postsynaptic density protein 95), also known as SAP-90 (synapse-associated protein 90), which is a protein that in humans is encoded by the DLG4 (discs large homolog 4) gene, and may be human PSD-95 (Uniprot: P78352).

A “subject in need thereof” refers to an individual who may benefit from the present invention. In one embodiment, said subject in need thereof is an individual suffering from an excitotoxicity-related disease and/or neuropathic pain. The subject to be treated is preferably a mammal, in particular a human being. Treatment of animals, such as mice, rats, dogs, cats, cows, horses, sheep and pigs, is, however, also within the scope of the present invention.

The terms “treatment” and “treating” as used herein refer to the management and care of a patient for the purpose of combating a condition, disease or disorder. The term is intended to include the full spectrum of treatments for a given condition from which the patient is suffering, and refer equally to curative therapy, prophylactic or preventative therapy and ameliorating or palliative therapy, such as administration of the peptide or composition for the purpose of: alleviating or relieving symptoms or complications; delaying the progression of the condition, partially arresting the clinical manifestations, disease or disorder; curing or eliminating the condition, disease or disorder; amelioration or palliation of the condition or symptoms, and remission (whether partial or total), whether detectable or undetectable; and/or preventing or reducing the risk of acquiring the condition, disease or disorder, wherein “preventing” or “prevention” is to be understood to refer to the management and care of a patient for the purpose of hindering the development of the condition, disease or disorder, and includes the administration of the active compounds to prevent or reduce the risk of the onset of symptoms or complications. The term “palliation”, and variations thereof, as used herein, means that the extent and/or undesirable manifestations of a physiological condition or symptom are lessened and/or time course of the progression is slowed or lengthened, as compared to not administering compositions of the present invention.

A “treatment effect” or “therapeutic effect” is manifested if there is a change in the condition being treated, as measured by the criteria constituting the definition of the terms “treating” and “treatment.” There is a “change” in the condition being treated if there is at least 5% improvement, preferably 10% improvement, more preferably at least 25%, even more preferably at least 50%, such as at least 75%, and most preferably at least 100% improvement. The change can be based on improvements in the severity of the treated condition in an individual, or on a difference in the frequency of improved conditions in populations of individuals with and without treatment with the bioactive agent, or with the bioactive agent in combination with a pharmaceutical composition of the present invention.

Polypeptides

In one aspect, the present invention relates to a polypeptide comprising the amino acid sequence of TX₁LETX₂X₃X₄GX₅X₆X₇PX₈TIRVX₉Q (SEQ ID NO: 1), wherein
X₁ is H, H-3Me or PyA-4;
X₂ is T, S, D or E;
X₃ is F, F-2-Br, F-2-Cl or F-3-F;
X₄ is W, NaI, or absent;
X₅ is D or N-Me-D;
X₆ is G, A or P;
X₇ is E or D;
X₈ is K or N-Me-K; and
X₉ is T or N-Me-T;

or a pharmaceutically acceptable salt thereof.

In one embodiment, the polypeptide of SEQ ID NO: 1 is covalently linked to a cyclization moiety. In one embodiment, the cyclization moiety comprises the amino acid sequence of pGX₁₀, wherein X₁₀ is C, Q or E. Hence, in one embodiment, the polypeptide comprises or consists of the amino acid sequence TX₁LETX₂X₃X₄GX₅X₆X₇PX₈TIRVX₉QpGX₁₀ (SEQ ID NO: 2), wherein
X₁ is H, H-3Me or PyA-4;
X₂ is T, S, D or E;
X₃ is F, F-2-Br, F-2-Cl or F-3-F;
X₄ is W, NaI, or absent;
X₅ is D or N-Me-D;
X₆ is G, A or P;
X₇ is E or D;
X₈ is K or N-Me-K;
X₉ is T or N-Me-T; and
X₁₀ is C, Q or E;

or a pharmaceutically acceptable salt thereof.

In one embodiment, X₄ is absent. Hence, in one embodiment, the polypeptide comprises or consists of the amino acid sequence TX₁LETX₂X₃GX₅X₆X₇PX₈TIRVX₉Q (SEQ ID NO: 3), wherein
X₁ is H, H-3Me or PyA-4;
X₂ is T, S, D or E;
X₃ is F, F-2-Br, F-2-Cl or F-3-F;
X₅ is D or N-Me-D;
X₆ is G, A or P;
X₇ is E or D;
X₈ is K or N-Me-K; and
X₉ is T or N-Me-T;

or a pharmaceutically acceptable salt thereof.

In one embodiment, the polypeptide comprises or consists of the amino acid sequence TX₁LETX₂X₃GX₅X₆X₇PX₈TIRVX₉QpGX₁₀ (SEQ ID NO: 4), wherein
X₁ is H, H-3Me or PyA-4;
X₂ is T, S, D or E;
X₃ is F, F-2-Br, F-2-Cl or F-3-F;
X₅ is D or N-Me-D;
X₆is G, A or P;
X₇is E or D;
X₈ is K or N-Me-K;
X₉ is T or N-Me-T; and
X₁₀ is C, Q or E;

or a pharmaceutically acceptable salt thereof.

In one embodiment, X₂ is T and X₃ is F. Hence, in one embodiment, the polypeptide comprises or consists of the amino acid sequence TX₁LETTFX₄GX₅X₆X₇PX₈TIRVX₉QpGX₁₀ (SEQ ID NO: 5), wherein
X₁ is H, H-3Me or PyA-4;
X₄ is W, NaI, or absent;
X₅is D or N-Me-D;
X₆is G, A or P;
X₇is E or D;
X₈ is K or N-Me-K;
X₉ is T or N-Me-T; and
X₁₀ is C, Q or E;

or a pharmaceutically acceptable salt thereof.

In one embodiment, X₂ is T, X₃ is F and X₄ is absent. Hence, in one embodiment, the polypeptide comprises or consists of the amino acid sequence TX₁LETTFGX₅X₆X₇PX₈TIRVX₉QpGX₁₀ (SEQ ID NO: 6), wherein
X₁ is H, H-3Me or PyA-4;
X₅is D or N-Me-D;
X₆is G, A or P;
X₇is E or D;
X₈ is K or N-Me-K;
X₉ is T or N-Me-T; and
X₁₀ is C, Q or E;

or a pharmaceutically acceptable salt thereof.

In one embodiment, X₂ is T, X₃ is F, X₅ is D, and X₆ is G. Thus, in one embodiment, the polypeptide comprises or consists of the amino acid sequence TX₁LETTFX₄GDGX₇PX₈TIRVX₉Q (SEQ ID NO: 7), wherein
X₁ is H, or PyA-4;
X₄ is W or NaI;
X₇ is E or D;
X₈ is K or N-Me-K; and
X₉ is T or N-Me-T;

or a pharmaceutically acceptable salt thereof.

In one embodiment, X₁ is H. In one embodiment, X₁ is H-3Me. In one embodiment, X₁ is PyA-4.

In one embodiment, X₂ is T. In one embodiment, X₂ is S. In one embodiment, X₂ is D. In one embodiment, X₂ is E.

In one embodiment, X₃ is F. In one embodiment, X₃is F-2-Br. In one embodiment, X₃ is F-2-Cl. In one embodiment, X₃ is F-3-F.

In one embodiment, X₄ is W. In one embodiment, X₄ is NaI.

In one embodiment, X₅ is D. In one embodiment, X₅ is N-Me-D.

In one embodiment, X₆ is G. In one embodiment, X₆ is A. In one embodiment, X₆ is P.

In one embodiment, X₇ is E. In one embodiment, X₇ is D.

In one embodiment, X₈ is K. In one embodiment, X₈ is N-Me-K.

In one embodiment, X₉ is T. In one embodiment, X₉ is N-Me-T.

In one embodiment, X₁₀ is C. In one embodiment, X₁₀ is Q. In one embodiment, X₁₀ is E.

In one embodiment, X₁ is H, X₂ is T, X₃ is F, X₄ is W, X₅ is D, and X₆ is G. In one embodiment, X₁ is H, X₂ is T, X₃ is F, X₄ is W, X₅ is D, X₆ is G, and X₇ is E. In one embodiment, X₁ is H, X₂ is T, X₃ is F, X₄ is W, X₅ is D, X₆ is G, and X₇ is D. In one embodiment, X₁ is H, X₂ is T, X₃ is F, X₄ is NaI, X₅ is D, X₆ is G, and X₇ is E. In one embodiment, X₁ is H, X₂ is PyA-4, X₃ is F, X₄ is W, X₅ is D, X₆ is G, and X₇ is E.

In one embodiment, the polypeptide comprises an amino acid sequence selected from the group consisting of: THLETTFWGDGE (SEQ ID NO: 8), THLETTFWGDGD (SEQ ID NO: 9), THLETTF(NaI)GDGE (SEQ ID NO: 10), and T(PyA-4)LETTFWGDGE (SEQ ID NO: 11).

In one embodiment, the polypeptide is a cyclic polypeptide. In one embodiment, the polypeptide is a cyclic polypeptide comprising or consisting of the amino acid sequence TX₁LETTFX₄GDGEPKTIRVTQpGX₁₀ (SEQ ID NO: 13)
wherein
X₁ is H or H-3Me;
X₄ is W or absent;
X₁₀ is C, Q or E;

or a pharmaceutically acceptable salt thereof.

In one embodiment, the polypeptide is a cyclic polypeptide comprising or consisting of the amino acid sequence THLETTFWGDGEPKTIRVTQ (SEQ ID NO: 419).

In one embodiment, the polypeptide is a cyclic polypeptide comprising or consisting of the amino acid sequence THLETTFGDGEPKTIRVTQ (SEQ ID NO: 420).

In one embodiment, the polypeptide is a cyclic polypeptide comprising or consisting of the amino acid sequence TH(3-Me)LETTFWGDGEPKTIRVTQ (SEQ ID NO: 421).

In one embodiment, the polypeptide is a cyclic polypeptide comprising or consisting of the amino acid sequence TH(3-Me)LETTFGDGEPKTIRVTQ (SEQ ID NO: 422).

In one embodiment, the polypeptide is cyclo-(THLETTFWGDGEPKTIRVTQpGE) (SEQ ID NO: 423). In one embodiment, the polypeptide is cyclo-(THLETTFGDGEPKTIRVTQpGE) (SEQ ID NO: 424). In one embodiment, the polypeptide is cyclo-(TH(3-Me)LETTFWGDGEPKTIRVTQpGE) (SEQ ID NO: 425). In one embodiment, the polypeptide is cyclo-(TH(3-Me)LETTFGDGEPKTIRVTQpGE) (SEQ ID NO: 426).

In one embodiment, the polypeptide is cyclo-(THLETTFWGDGEPKTIRVTQpGQ) (SEQ ID NO: 14). In one embodiment, the polypeptide is cyclo-(THLETTFGDGEPKTIRVTQpGQ) (SEQ ID NO: 15). In one embodiment, the polypeptide is cyclo-(TH(3-Me)LETTFWGDGEPKTIRVTQpGQ) (SEQ ID NO: 16). In one embodiment, the polypeptide is cyclo-(TH(3-Me)LETTFGDGEPKTIRVTQpGQ) (SEQ ID NO: 17).

In one embodiment, the polypeptide comprises at least 20 amino acid residues, such as at least 21 amino acid residues, such as at least 22 amino acid residues, such as at least 23 amino acid residues, such as at least 24 amino acid residues, such as at least 25 amino acid residues, such as at least 26 amino acid residues, such as at least 27 amino acid residues, such as at least 28 amino acid residues, such as at least 29 amino acid residues, such as at least 30 amino acid residues, such as at least 31 amino acid residues, such as at least 32 amino acid residues, such as at least 33 amino acid residues, such as at least 34 amino acid residues, such as at least 35 amino acid residues, such as at least 36 amino acid residues, such as at least 37 amino acid residues.

In one embodiment, the polypeptide comprises no more than 50 amino acid residues, such as no more than 45 amino acid residues, such as no more than 40 amino acid residues, such as no more than 35 amino acid residues, such as no more than 30 amino acid residues, such as no more than 29 amino acid residues, such as no more than 28 amino acid residues, such as no more than 27 amino acid residues, such as no more than 26 amino acid residues, such as no more than 25 amino acid residues, such as no more than 24 amino acid residues, such as no more than 23 amino acid residues, such as no more than 22 amino acid residues, such as no more than 21 amino acid residues, such as no more than 20 amino acid residues.

In one embodiment, the polypeptide comprises in the range of 19 to 50 amino acid residues, such as in the range of 19 to 45 amino acid residues, such as in the range of 19 to 40 amino acid residues, such as in the range of 19 to 35 amino acid residues, such as in the range of 19 to 30 amino acid residues, such as in the range of 19 to 25 amino acid residues, such as in the range of 19 to 23 amino acid residues, such as in the range of 20 to 23 amino acid residues, such as in the range of 20 to 22 amino acid residues.

Cyclic Polypeptides

In a preferred embodiment, the polypeptide is cyclized to form a cyclic polypeptide. For example, a polypeptide may be cyclized by side chain-to-side chain, tail-to-side chain, side chain-to-head and head-to-tail. Common cyclization strategies include, but are not limited to, disulfide bridge between two cysteines (side chain-to-side chain), thioether bridge with e.g. a bromoacetic addition on the N-terminus and a cysteine (head-to-side chain) and lactamization either using coupling between a basic residue (Lys) and acid residues (Asp or Glu), or via native chemical ligation (NCL). Most of these strategies employ quasi-orthogonal protecting groups to Fmoc and tBu/Boc such as trityl (Trt) or allyloxycarbonyl (Alloc) on Lys, 4-monomethoxytrityl (Mmt) on Cys, allyl (All) or 2-phenylisopropyl (2-PhiPr) esters on Asp or Glu to selectively deprotect an amino group, thiol and carboxylate, respectively.

As used herein, the term “head-to-tail cyclized peptide” is used interchangeably with the term “backbone cyclized peptide”. In one embodiment, the cyclic peptide is a backbone cyclized peptide. In one embodiment, the cyclic peptide is formed by the formation of an amine bond between its N-terminus- and its C-terminus-parts, i.e. head-to tail cyclization.

In some embodiments, a rink amide resin is used in the preparation of the cyclic polypeptide, see Examples 1 and 4. Hence, when the polypeptide is cleaved off from the resin, the E amino acid residue at position X₁₀ is converted to a Q amino acid residue.

In one aspect, the present invention relates to a cyclic polypeptide comprising the amino acid sequence of LETX₂X₃X₄GX₅X₆X₇ (SEQ ID NO: 436), wherein
X₂ is T, S, D or E;
X₃ is F, F-2-Br, F-2-Cl or F-3-F;
X₄ is W, NaI, or absent;
X₅ is D or N-Me-D;
X₆ is G, A or P; and
X₇ is E or D;

or a pharmaceutically acceptable salt thereof.

In one embodiment, the cyclic polypeptide comprises or consist of the polypeptide as described herein. In one embodiment, the cyclic polypeptide comprises in the range of 19 to 50 amino acid residues, such as in the range of 20 to 22 amino acid residues.

In one embodiment, the cyclic peptide comprises or consistis of the amino acid sequence of TX₁LETX₂X₃X₄GX₅X₆X₇PX₈TIRVX₉Q (SEQ ID NO: 1), wherein
X₁ is H, H-3Me or PyA-4;
X₂ is T, S, D or E;
X₃ is F, F-2-Br, F-2-Cl or F-3-F;
X₄ is W, NaI, or absent;
X₅ is D or N-Me-D;
X₆ is G, A or P;
X₇ is E or D;
X₈ is K or N-Me-K; and
X₉ is T or N-Me-T;

or a pharmaceutically acceptable salt thereof.

In one embodiment, the cyclic polypeptide comprises an amino acid sequence selected from the group consisting of: THLETTFWGDGE (SEQ ID NO: 8), THLETTFWGDGD (SEQ ID NO: 9), THLETTF(NaI)GDGE (SEQ ID NO: 10), and T(PyA-4)LETTFWGDGE (SEQ ID NO: 11).

In one embodiment, the polypeptide comprises an amino acid sequence selected from the group consisting of SEQ ID NO: 14 to 136 as defined herein. In one embodiment, the polypeptide consists of an amino acid sequence selected from the group consisting of SEQ ID NO: 14 to 136. In one embodiment, the polypeptide comprises an amino acid sequence selected from the group consisting of SEQ ID NO: 139 to 433 as defined herein. In one embodiment, the polypeptide consists of an amino acid sequence selected from the group consisting of SEQ ID NO: 139 to 433. The expression “the group consisting of SEQ ID NO: 139 to 433” includes each and every sequence with a SEQ ID NO of 139 to 433. Analogiusly, the expression “the group consisting of SEQ ID NO: 1 to 5” includes SEQ ID NO: 1, SEQ ID NO: 2, SEQ ID NO: 3, SEQ ID NO: 4 and SEQ ID NO: 5.

Salts and Prodrugs

The polypeptide as defined herein can be in the form of a pharmaceutically acceptable salt or prodrug of said polypeptide. In one embodiment of the present invention, the polypeptide as defined herein can be formulated as a pharmaceutically acceptable addition salt or hydrate of said compound, such as but not limited to K⁺, Na⁺, as well as non-salt e.g. H⁺.

Affinity for PSD-95

In one embodiment, the polypeptide is capable of binding to PSD-95. In one embodiment, the polypeptide binds to PSD-95-PDZ2 with a K_{d} value of less than 100 μM, such as less than 75 μM, such as less than 50 μM, such as less than 25 μM, such as less than 20 μM, such as less than 15 μM, such as less than 10 μM, such as less than 5 μM, such as less than 4 μM, such as less than 3 μM, such as less than 2 μM, such as less than 1 μM. Said K_{d} value may be determined using a fluorescence polarization (FP) assay or an isothermal titration calorimetry (ITC) assay as described in Example 1.

In one embodiment, the polypeptide is capable of inhibiting binding of nNOS to the PDZ2 domain of PSD-95. In one embodiment, the compound has a Kᵢ value for inhibiting binding of nNOS to PDZ2 domain of PSD-95 of less than 100 μM, such as less than 75 μM, such as less than 50 μM, such as less than 10 μM, such as less than 5 μM, such as less than 2.5 μM, such as less than 1 μM. Said Kᵢ value may be determined using a fluorescence polarization (FP) competition assay. Said K_{d} value may be determined using a fluorescence polarization (FP) assay or an isothermal titration calorimetry (ITC) assay as described in Example 1.

Membrane Permeability

Since PSD-95 is located intracellularly, it is essential for any drug targeting PSD-95 to efficiently cross the cell membrane. To assess the cellular permeability and delivery to the cytosol of the compounds as defined herein, the cellular chloroalkane penetration assay (CAPA) may be used (Peraro et al. 2018). This assay takes advantage of a modified haloalkane dehalogenase designed to covalently bind chloroalkane (CA) molecules. A HeLa cell line expressing a fusion protein comprising a HaloTag, a green fluorescent protein (GFP) and a mitochondria-targeting peptide is used to report cytosolic delivery. The general format of the CAPA is a pulse-chase assay (Deprey & Kritzer, 2020). Cells expressing the HaloTag enzyme are incubated with CA-tagged peptides. When these CA-peptides penetrate the cell membrane and reach the cytosol, they will bind to and react with the HaloTag (pulse step). Following a washing step, the cells are incubated with a CA-tagged dye that quantitatively penetrates the cell membrane and reacts with remaining unreacted HaloTag sites (chase step). Flow cytometry is used to measure the fluorescence intensity of the cells and the measured fluorescence is inversely proportional to the amount of CA-peptides that reach the penetrated the cells and can thus be used to assess cytosolic delivery. The obtained data is commonly expressed as CP₅₀ values, the concentration at which 50% cell penetration is observed. The CP₅₀ value of a compound may be measured as described in example 16. Example 16 shows that the cellular uptake of described cyclic peptide exhibited suitable cellular uptake for medical applications.

In one embodiment, the compound has a CP₅₀ value of no more than 250 μM, such as no more than 200 μM, such as no more than 150 μM, such as no more than 100 μM, such as no more than 80 μM, such as no more than 70 μM, such as no more than 60 μM, such as no more than 50 μM, such as no more than 40 μM, such as no more than 30 μM, such as no more than 20 μM, such as no more than 15 μM, such as no more than 10 μM, such as no more than 5 μM. Preferably, the compound has a CP₅₀ value of no more than 60 μM.

Plasmin Stability

Ischemic stroke (also referred to as ‘brain ischemia’ or ‘cerebral ischemia’) is usually caused by a blockage in an artery that supplies blood to the brain. The blockage reduces the blood flow and oxygen to the brain, leading to damage or death of brain cells. The blockage of the blood vessels can be removed using a range of mechanical devices, or using “clot busting agents” which are delivered intravenously or intra-arterially. Among such clot busting agents is Tissue plasminogen factor (tPA), which generates plasmin from plasminogen. Examples of recombinant tPA's are alteplase, reteplase and tenecteplase, and other thrombolytic drugs that break down clots include streptokinase, urokinase and desmotaplase.

In one embodiment, the polypeptides of the present invention are administered to subjects receiving tPA or a recombinant tPA, which is the standard-of-care for AIS.

Thus, it is essential that the polypeptide is compatible with the administration of tPA, including the generation of plasmin, which is a serine protease.

The in vitro plasmin stability of polypeptides of the present invention were determined in example 15. In one embodiment, the compound has a half-life in the plasmin stability assay described in example 15 of at least 10 min in the presence of plasmin, such as at least 30 min, such as at least 1 h, such as at least 2 h, such as at least 3 h, such as at least 4 h, such as at least 5 h, such as at least 6 h, such as at least 7 h, such as at least 8 h, such as at least 9 h, such as at least 10 h, such as at least 15 h, such as at least 20 h, such as at least 30 h.

Polypeptide Modifications

In one embodiment, the polypeptide is further modified by glycosylation, PEGylation, amidation, esterification, acylation, acetylation and/or alkylation. In one embodiment, one or more of the amino acid residues in the polypeptide are alkylated, such as methylated. For example, X₅ is may be N-Me-D, X₈ may be N-Me-K and/or X₉ may be N-Me-T. In one embodiment, the polypeptide is further conjugated to a moiety. In one embodiment, said moiety is selected from the group consisting of PEG, monosaccharides, fluorophores, chromophores, radioactive compounds, and cell penetrating peptides. In one embodiment, the moiety is a detectable moiety. In one embodiment, the polypeptide of SEQ ID NO: 1 is covalently linked to a cyclization moiety. In one embodiment, the cyclization moiety comprises the amino acid sequence of pGX₁₀, wherein X₁₀ is C, Q or E. In one embodiment, the polypeptide is conjugated to a chloroalkane tag (CA), which has the structure of:

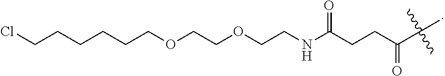

Polynucleotides, Vectors and Cells

In one aspect of the present invention there is provided a nucleic acid construct encoding for and being capable of expressing a peptide comprising an amino acid sequence as defined herein. By nucleic acid construct is understood a genetically engineered nucleic acid. The nucleic acid construct may be a non-replicating and linear nucleic acid, a circular expression vector or an autonomously replicating plasmid. In one aspect, the present invention concerns a polynucleotide encoding the corresponding linear sequence of the cyclic peptide as defined herein. In one aspect, the present invention concerns a vector comprising said polynucleotide. In one aspect, the present invention concerns a host cell comprising said polynucleotide or said vector. In one embodiment, the host cell is a bacterial cell. In one embodiment, the host cell is a mammalian cell. In one embodiment, the host cell is a human cell.

Method of Preparation of Polypeptides

The polypeptides according to the present invention may be prepared by any methods known in the art. Thus, the polypeptides may be prepared by standard peptide-preparation techniques, such as solution synthesis or Merrifield-type solid phase synthesis.

In one embodiment, a polypeptide according to the invention is synthetically made or produced. The methods for synthetic production of peptides are well known in the art. Detailed descriptions as well as practical advice for producing synthetic ploypeptides may be found in Synthetic Peptides: A User's Guide (Advances in Molecular Biology), Grant G. A. ed., Oxford University Press, 2002, or in: Pharmaceutical Formulation: Development of Peptides and Proteins, Frokjaer and Hovgaard eds., Taylor and Francis, 1999. In one embodiment, the polypeptide or polypeptide sequences of the invention are produced synthetically, in particular, by the Sequence Assisted Peptide Synthesis (SAPS) method, by solution synthesis, by Solid-phase peptide synthesis (SPPS) such as Merrifield-type solid phase synthesis, by recombinant techniques (production by host cells comprising a first nucleic acid sequence encoding the polypeptide operably associated with a second nucleic acid capable of directing expression in said host cells) or enzymatic synthesis. These are well-known to the skilled person.

After purification of the linear polypeptides, such as by reversed phase HPLC, the linear polypeptides are further processed to cyclic peptides. Techniques for cyclizing a polypeptide and for obtaining a cyclic polypeptide, for example by using a solid support, are well known by the man skilled in the art.

In one aspect, the present invention concerns a method of manufacturing a polypeptide as defined herein, the method comprising the step of recombinantly expressing or synthetically producing the polypeptide. In one aspect, the present invention concerns a method of manufacturing a cyclic polypeptide as defined herein, the method comprising the steps of recombinantly expressing or synthetically producing the corresponding linear polypeptide followed by cyclisation.

In one aspect, the present invention concerns a method for manufacturing the polypeptide as defined herein, said method comprising the steps of:
a) preparing a peptide using Fmoc/tBu-based solid-phase peptide synthesis (SPPS), and
b) cyclization of said peptide via native chemical ligation (NCL).

In one embodiement, SPPS and NCL are conducted as outlined in Example 1. In one embodiment, step b) involves oxidizing a C-terminal hydrazine group to an azide and reacting said azide with a thiol group of the N-terminal Cys, followed by transthioesterification to form an amide bond linkage.

In one embodiment, the method for manufacturing further comprises a step following step b) wherein a fluorophore is conjugated to the polypeptide.

In one aspect, the present invention concerns a method for manufacturing the polypeptide as defined herein, said method comprising the steps of:
a) Providing a cellulose membrane;
b) Coupling of PEG spacer and adding a mixture of Fmoc/Boc-Gly to the cellulose membrane provided in step a);
c) Capping the membrane prepared in step b) with acetic anhydride;
d) Adding quasi-orthogonal protected AA to the product of step c);
e) Preparing the remaining polypeptide using Fmoc/tBu-based solid-phase peptide synthesis (SPPS) on the AA of step d);
f) Removing the quasi-orthogonal protecting group from the polypeptide generated in step e) and cyclizing the polypeptide;
g) Cleaving side-chain protecting groups from the polypeptide generated in step f); and
h) Cleaving the polypeptide from the cellulose membrane.

In one embodiment, the polypeptide is prepared by SPOT peptide array synthesis as described in Example 1 and further cleaving the polypeptide from the cellulose membrane.

In one embodiment, the synthesis of cyclic polypeptide as defined herein is conducted on a resin, such as a cellulose membrane. The synthesis may then be initiated with the addition a mixture of Fmoc/Boc-Gly to decrease the membrane loading thus decreasing the concentration of the individual peptide spots, which will also lower the risk of non-specific binding to target protein. Subsequently, the membrane is capped with acetic anhydride, so the only functional parts present of the membranes (spots) are those primed with Fmoc-Gly mixture. After the Fmoc group removal, the capping efficacy may be qualitatively controlled by bromophenol blue (BPP). In continuation, after the Fmoc group removal, the AA (e.g. Cys, Glu or Asp) with the quasi-orthogonal protecting group is coupled. The rest of the AAs of the peptides are coupled following standard Fmoc routine. Upon the peptide synthesis completion, the orthogonal protecting group is removed to ‘free’ the functional (e.g. carboxyl group) group, which is cyclized to the deprotected N-terminal. Once the peptide is cyclized, the membrane is treated with TFA and a scavenger mixture in order to remove the temporary side chain protecting groups. Further, the generated peptide may be cleaved off from the resin.

Medical Use

In one aspect, the present invention relates to a polypeptide, a composition, a polynucleotide, vector, or a host cell as defined herein for use as a medicament. In one embodiment, the present invention relates to a cyclic polypeptide as defined herein for use as a medicament.

In one aspect, the present invention relates to a method of preventing and/or treating an excitotoxicity-related disease and/or neuropathic pain, said method comprising administering a therapeutically effective amount of the polypeptide, the composition, the polynucleotide, the vector, or the host cell as defined herein to a subject in need thereof.

In one aspect, the present invention relates to use of the polypeptide, the composition, the polynucleotide, the vector, or the host cell as defined herein for the manufacture of a medicament for the treatment and/or prevention of an excitotoxicity-related disease and/or neuropathic pain in a subject.

In one embodiment, the subject as referred to herein is a mammal, such as a human.

Excitotoxic-Related Diseases

The polypeptides of the present invention are PSD-95 inhibitors and are thus able to inhibit excitotoxicity. Hence, the compounds of the present invention are useful in treating a variety of diseases, particularly neurological diseases, and especially diseases mediated in part by excitotoxity. In one aspect, the present invention relates to a polypeptide, a composition, a polynucleotide, vector, or a host cell as defined herein for use in prevention and/or treatment of an excitotoxic-related disease in a subject. In one embodiment, the present invention relates to a cyclic polypeptide as defined herein for use in prevention and/or treatment of an excitotoxic-related disease in a subject.

The ternary complex between the N-methyl-D-aspartate receptor (NMDAR), Postsynaptic density protein-95 (PSD-95) and neuronal nitric oxide synthase (nNOS) plays an important role in the excitotoxicity mechanism of cell death. As the polypeptides of the present invention are capable of inhibiting binding of nNOS to PSD-95, and thus prevent/interrupt formation of excess NO causing excitotoxixity, the polypeptides may be useful in the treatment of excitotoxic-related diseases.

A large number of indications such as ischemia, trauma, epilepsy and chronic neurodegenerative disorders have been linked to excitotoxicity (Gardoni, F. et al., 2006, European Journal of Pharmacology, 545, 2-10). In one embodiment the excitotoxic-related disease is stroke, such as ischemic stroke. In one embodiment, the excitotoxic-related disease is ischemic or traumatic injury of the CNS, such as spinal cord injury and traumatic brain injury. In one embodiment, the excitotoxic-related disease is epilepsy. In one embodiment, the excitotoxic-related disease is a neurodegenerative disease of the CNS. In one embodiment, the neurodegenerative disease of the CNS is selected from the group consisting of Alzheimer's disease, Huntington's disease and Parkinson's disease.

In one aspect, the present invention relates to a polypeptide as defined herein for use in preventing, treating, reducing and/or delaying development of an excitotoxic-related disease. In one embodiment, the excitotoxic-related disease is stroke. In one embodiment, the excitotoxic-related disease is ischemic stroke. In one embodiment, the excitotoxic-related disease is cerebral ischemia. In one embodiment, the excitotoxic-related disease is acute ischemic stroke. In one embodiment, the excitotoxic-related disease is subarachnoid hemorrhage.

In one aspect, the present invention relates to use of a polypeptide as defined herein for the manufacture of a medicament for preventing, treating, reducing and/or delaying development of an excitotoxic-related disease.

In one aspect, the present invention relates to a method for preventing, treating, reducing and/or delaying development of an excitotoxic-related, said method comprising administering a therapeutically effective amount of polypeptide as defined herein.

In one aspect, the present invention relates to a polypeptide as defined herein for use in reducing and/or protecting against a damaging effect of excitotoxicity. In one embodiment, the polypeptide is for use in reducing the damaging effect of stroke. In one embodiment, the polypeptide is for use in treating a damaging effect of acute ischemic stroke. In one embodiment, the polypeptide is for use in treating a damaging effect of subarachnoid hemorrhage.

In one aspect, the present invention relates to a method for protecting against and/or reducing the damaging effect of excitotoxicity to the brain or spinal cord in a subject, said method comprising the step of administering an effective amount of a polypeptide as defined herein to the subject to protect against and/or reduce the damaging effect.

In one aspect, the present invention relates to a method of treating, reducing, or delaying development of a condition mediated by excitotoxicity comprising administering a polypeptide as defined herein to a human subject having or at risk of the condition.

In one aspect, the present invention relates to a method of treating or inhibiting or delaying at least one sign or symptom of a condition mediated by excitotoxicity in a subject, comprising administering a polypeptide as defined herein to the subject having the conditions, or a risk factor associated with the condition. In one embodiment, said condition is stroke or traumatic injury to the CNS. In one embodiment, the excitotoxic-related disease is ischemic or traumatic injury to/in/of the CNS.

In one aspect, the present invention relates to a method of reducing the damaging effect of stroke in a subject having stroke, comprising administering to the subject an effective amount of a polyppetide as defined herein to reduce the damaging effect of the stroke.

As used herein, “stroke” is a general term that refers to conditions caused by the occlusion or hemorrhage of one or more blood vessels supplying the brain, leading to cell death. “Ischemic stroke”, as used herein, refers to stroke caused by an occlusion of one or more blood vessels supplying the brain. Types of ischemic stroke include, e.g., embolic stroke, cardioembolic stroke, thrombotic stroke, large vessel thrombosis, lacunar infarction, artery-artery stroke and cryptogenic stroke. “Cerebral ischemia” is a condition in which a blockage in an artery restricts the delivery of oxygen-rich blood to the brain, resulting in damage to brain tissue. Cerebral ischemia is sometimes called brain ischemia or cerebrovascular ischemia.

“Hemorrhagic stroke”, as used herein, refers to stroke caused by hemorrhage of one or more blood vessels supplying the brain. Types of hemorrhagic stroke include, e.g., subdural stroke, intraparenchymal stroke, epidural stroke and subarachnoid stroke.

In one embodiment, the disease treatable by the compound of the present invention is ischemic or traumatic injury of the CNS. In one aspect, the present invention relates to a method of reducing the damaging effect of traumatic injury or ischemia to the brain or spinal cord in a subject, said method comprising treating said subject with a polypeptide as defined herein to effect said reduction.

In one aspect, the present invention relates to a method of inhibiting cerebral ischemia due to endovascular surgery, comprising administering to a subject undergoing endovascular surgery a polypeptide as defined herein in a regime effective to inhibit cerebral ischemia.

In one aspect, the present invention relates to a method of inhibiting ischemic damage from endovascular surgery to treat an aneurysm, diagnostic angiography or carotid stenting comprising administering an effective regime of a polypeptide as defined herein to a subject undergoing endovascular surgery to treat an aneurysm or diagnostic angiography.

In one aspect, the present invention relates to a polypeptide as defined herein for use in inhibiting ischemic damage from neurosurgery. In one embodiment, said neurosurgery is diagnostic angiography of the brain or endovascular surgery to treat an aneurysm.

In some embodiments, the polypeptide is administered in combination with reperfusion therapy. In one embodiment, the polypeptide and the reperfusion are administered simultaneously, sequentially or separately to the subject.

The term ‘reperfusion therapy’ as used herein refers to a medical treatment to restore blood flow, either through or around, blocked arteries. Reperfusion therapy includes medical agents and mechanical reperfusion. Said medical agents may be thrombolytics or fibrinolytics used in a process called thrombolysis. In some embodiments, reperfusion therapy is performed by administering a thrombolytic agent, such as a plasminogen activator, for example tPA. In one embodiment, the polypeptide as defined herein is administered in combination with a plasminogen activator, for example tPA.

In some embodiments, the reperfusion therapy is mechanical reperfusion including surgery. Surgeries performed may be minimally-invasive endovascular procedures. Among mechanical reperfusion devices, there are intra-arterial catheters, balloons, stents, and various clot retrieval devices.

In one embodiment, the polypeptide is administered in combination with a thrombolytic agent, and the compound and the thrombolytic agent are administered simultaneously, sequentially or separately to the subject.

In one aspect, the present invention relates to a method of treating a damaging effect of ischemia on the central nervous system, comprising
a) administering a polypeptide as defined herein to a subject having or at risk of ischemia, and
b) performing reperfusion therapy on the subject,

wherein the polypeptide and reperfusion therapy treat a damaging effect of the ischemia on the central nervous system of the subject.

In one aspect, the present invention relates to a polypeptide as defined herein for use in treating a damaging effect of ischemia on the central nervous system in a subject having or at risk of ischemia, wherein reperfusion therapy is performed on the subject, and the polypeptide and reperfusion therapy treat a damaging effect of the ischemia on the central nervous system of the subject.

In one embodiment, the method further comprising administering a thrombolytic agent simultaneously, sequentially or separately to the subject.

In one aspect, the present invention relates to a kit of parts comprising at least two separate unit dosage forms (A) and (B), wherein
(A) comprises a polypeptide as defined herein; and
(B) comprises a thrombolytic agent.

In one aspect, the kit of parts as defined herein is for use in the treatment of a damaging effect of ischemia on the central nervous system, wherein (A) and (B) are administered simultaneously, sequentially or separately to the subject.

In one aspect, the present invention relates to a polypeptide as defined herein for use in treating a damaging effect of subarachnoid hemorrhage. The term “subarachnoid hemorrhage” as used herein refers to a hemorrhage state in a subarachnoid cavity.

In one aspect, the present invention relates to a method of treating a subarachnoid hemorrhage in a subject, comprising administering a polypeptide as defined herein to a subject having a subarachnoid hemorrhage, wherein development of neurocognitive deficits in the subject is inhibited.

In one aspect, the present invention relates to a method of inhibiting development of a neurologic or neurocognitive deficit of subarachnoid hemorrhage in a subject, comprising administering a polypeptide as defined herein to a subject having a subarachnoid hemorrhage, wherein development of a neurologic or neurocognitive deficit in the subject is inhibited.

Neuropathic Pain

Other neurological diseases treatable by the polypeptides of the present invention not known to be associated with excitotoxicity include anxiety and pain. In one aspect, the present invention relates to a polypeptide, a composition, a polynucleotide, a vector, or a host cell as defined herein for use in prevention and/or treatment of neuropathic pain in a subject. In one embodiment, the present invention relates to a cyclic polypeptide as defined herein for use in prevention and/or treatment of neuropathic pain in a subject.

Neuropathic pain is a category of pain that includes several forms of chronic pain and which results from dysfunction of nervous rather than somatic tissue. Neuropathic pain, that is pain deriving from dysfunction of the central or peripheral nervous system, may also be a consequence of damage to peripheral nerves or to regions of the central nervous system, may result from disease, or may be idiopathic. Symptoms of neuropathic pain include sensations of burning, tingling, electricity, pins and needles, paresthesia, dysesthesia, stiffness, numbness in the extremities, feelings of bodily distortion, allodynia (pain evoked by stimulation that is normally innocuous), hyperalgesia (abnormal sensitivity to pain), hyperpathia (an exaggerated pain response persisting long after the pain stimuli cease), phantom pain, and spontaneous pain.

PSD-95 has been demonstrated to be involved in the central mechanisms of neuropathic pain (Tao, F. et al., 2003, Neuroscience, 731-739; Florio, S. K. et al., 2009, British Journal of Pharmacology, 158, 494-506). As the polypeptides of the present invention inhibit PSD-95, the polypeptides may be useful in the treatment of neuopathic pain.

Administration

According to the present invention, a peptide, or a composition comprising a peptide as defined herein, is administered to individuals in need of treatment in pharmaceutically effective doses or a therapeutically effective amount. The dosage requirements will vary with the particular drug composition employed the route of administration and the particular subject being treated, which depend on the severity and the sort of the disorder as well as on the weight and general state of the subject. It will also be recognized by one skilled in the art that the optimal quantity and spacing of individual dosages of a peptide compound will be determined by the nature and extent of the condition being treated, the form, route and site of administration, and the particular patient being treated, and that such optima can be determined by conventional techniques. It will also be appreciated by one of skill in the art that the optimal course of treatment, i.e., the number of doses of a compound given per day for a defined number of days, can be ascertained using conventional course of treatment determination tests.

Pharmaceutical Composition

Whilst it is possible for the polypeptides of the present invention to be administered as the raw peptide, it is preferred to present them in the form of a pharmaceutical formulation. Accordingly, the present invention further provides a pharmaceutical formulation, which comprises polypeptide of the present invention or a pharmaceutically acceptable salt thereof, and a pharmaceutically acceptable carrier therefore. Thus, in one aspect, the present invention concerns a composition, such as a pharmaceutical composition, comprising the polypeptide as defined herein. The pharmaceutical formulations may be prepared by conventional techniques, e.g. as described in Remington: The Science and Practice of Pharmacy 2005, Lippincott, Williams & Wilkins.

### EXAMPLES

### Example 1

### Materials and Methods

Solid phase peptide synthesis (SPPS). Peptides were synthesized by employing the the 9-Fluoromethyl (Fmoc)/tert-butyl (tBu) strategy.

Linear peptides were synthesized with preloaded Fmoc-Gly or Fmoc-Val-Wang resin (100-200 mesh). Reagents were prepared as solutions in N,N-Dimethyl-formamide (DMF). For cyclic peptide synthesis, rink Amide-ChemMatrix® resin was preloaded with the quasi-orthogonal building blocks for cyclization (Fmoc-Glu(PP)-OH, Fmoc-Glu-PP, Fmoc-Asp(PP)-OH and Fmoc-Cys(Mmt)-OH). Hence, a solution of 1.5 eq of the selected building block, 4 eq of N,N′-Diisopropylcarbodiimide (DIC) and 4 eq of Oxyma Pure (Novabiochem®) were stored 3 h at room temperature with continuous shaking. Afterwards, the excess of reagents was removed and the resin was capped with a solution of 20 eq of N,N-diisopropylethylamine (DI PEA) and 20 eq of acetic anhydride. Resins were dried under vacuum conditions for storage. SPPS was performed with 4 eq of Fmoc-AA-OH, 4 eq of DIC and 4 eq of Oxyma Pure during 1 h at room temperature. Deprotection of the Fmoc group was performed with 20% piperidine in DMF during 10 min. When the linear peptide synthesis on-resin was completed, the resin was dried under vacuum conditions for 3 h. Resins with peptides containing a quasi-orthogonal protective group were treated with 95% dichloromethane (DCM), 3% triisopropylsilane (TIPS) and 2% trifluoroacetic acid (TFA) for 20 min 4 times to ensure the removal of the quasi-orthogonal protecting group. Afterwards, the resin was washed 5 times with 5 mL of DCM and neutralized with 5% DIPEA in DCM. Then, the resin was washed 5 times with DMF, and the peptide bound to resin was cyclized with a solution of 4 eq of (7-azabenzotriazol-1-yloxy)tripyrrolidinophosphonium hexafluorophosphate (PyAOP, Iris Biotech) and 4 eq of DIPEA, in DMF for 3 h or overnight. The resin was then dried for 3 h.

CA-tagged peptides were prepared using quasi-orthogonal Fmoc-Lys(Alloc)-OH. Following successful cyclization, quasi-orthogonal protective groups were treated with 0.2 eq Pd(PPh₃)₄ and 20 eq PhSiH₃ in DCM for 2×15 min. Following complete removal of the Alloc protective group, the side chain amine of the deprotected Lys residue was functionalized with chloroalkane tag (CA). The CA tag was coupled to the nitrogen group of the Lys of cyclized peptides using a mixture of CA:PyBOP:DIPEA in DMF (3:3:10) for 16 h.

Linear and cyclic peptides were cleaved from the resin using a cleavage cocktail containing 95% TFA, 2.5% H₂O and 2.5% TIPS for 3 h. TFA removal and precipitation of the peptide was performed with cold ether. Peptides were then dissolved in acidified MQ H₂O (0.1% TFA) and purified using reverse phase high performance liquid chromatography (RP-HPLC), with a Waters prep 150LC system and a reverse phase column (Zorbax 300 SB-C18, 21.2 mm×250 mm) in a linear gradient from 5% to 35% B over 30 min. A binary solvent system [A: H₂O/TFA 99.9/0.1 and B: acetonitrile (MeCN)/TFA 99.9/0.1] was used. The final products were lyophilized. After purification, the peptides were analyzed once again by mass-spectrometry with a Waters Acquity UPLC system with a QDa mass detector module, using a linear gradient of 5 to 60% B over 6 min. [A: H₂O/TFA 99.9/0.1 and B: MeCN/TFA 99.9/0.1].

Native chemical ligation (NCL) cyclization. The hydrazide peptides for NCL were synthesized by preloading 2-Cl-trityl resin with 4 eq of 9-fluorenylmethyl carbazate, 4 eq of DIC and 4 eq of Oxyma Pure overnight. The rest of the peptide sequence was then synthesized and purified using the procedure described in SPPS section above. The final purified linear peptide hydrazide was then dissolved in a buffer containing 6 M guanidinium chloride (GnHCl) and 0.2 M phosphate buffered saline (PBS), pH 3.0 in a salt-ice bath. Then, 10 eq of sodium nitrite were added to the peptide solution to oxidize the peptide hydrazide. The solution was left reacting for 30 min. Afterwards, the solution was brought to pH 6.8 using a 0.1 M solution of NaOH at room temperature and 100 eq of 4-mercaptophenylacetic acid (MPAA) were eventually added to the mixture to form the peptide thioester. The solution was left reacting at room temperature for 2 h to allow the peptide cyclization completion. After 2 h, the solution was diluted with acidified H₂O and purified using reverse phase high performance liquid chromatography (RP-HPLC) with a Waters prep 150LC system and a reverse phase column (Zorbax 300 SB-C18, 21.2 mm×250 mm) in a linear gradient from 10% to 40% B during 30 min. using a binary solvent system [A: H2O/TFA 99.9/0.1 and B: MeCN/TFA 99.9/0.1]. The final product was lyophilized. After purification, the peptides were analyzed by mass-spectrometry with a Waters ACQUITY UPLC system with a QDa mass detector module and using a linear gradient of 5 to 60% B over 6 min. [A: H₂O/TFA 99.9/0.1 and B: MeCN/TFA 99.9/0.1].

Protein expression. The pRSET plasmids encoding the (7× His)-PSD-95-PDZ2 wild type and (7× His)-PSD-95-PDZ2-V178C sequences were obtained as described previously.³⁸ The DNA constructs encoding the PSD-95-PDZ2 mutants (K165A, K168A, F172A, F1721, S173A, N180A, T192A, K193A, H225A, E226A, V229A K233A) were produced using Phusion® site-directed mutagenesis kit on the pRSET plasmid of the wild type PSD-95-PDZ2 and with the primers listed in Table 1. The PSD-95-PDZ2 mutants were transformed and expressed as previously described,³⁸ in Escherichia coli B21 pLys cells at 37° C. and 0.5 mM isopropyl-D-thiogalactopyranoside (IPTG). After 2 hours of expression at 37° C., the cells were harvested and lysed using B-PER bacterial protein extraction reagent. Proteins were purified using a His-tag column equilibrated with wash buffer (50 nM NaPi, 20 mM imidazole) and eluted with elution buffer (50 mM NaPi, 50 mM NaCl, 250 mM imidazole). Afterwards, the proteins were further purified by size exclusion purification with an Akta Explorer 100 Air, with a HiLoad 16/600 Superdex 75 pg prepacked column, using a buffer containing 50 mM NaPi, 50 mM NaCl in a flow rate of 1 mL min⁻¹. Protein concentration was measured by NanoDrop 1000 and mass determination of proteins was analyzed using an Agilent 6410 triple quadrupole LC-MS with a Poroshell column, 300SB-C18 2.1×75 mm in a linear gradient of 5% to 60% B, using a binary solvent system [A: H₂O/MeCN/TFA, 94.9/5/0.1 and B: H₂O/MeCN/TFA, 5/94.9/0.1] with a flow of 0.75 mL min⁻¹. Protein mass was deconvoluted using Agilent Mass Hunter software. Final protein purity assessment was performed using a Waters ACQUITY UPLC with BEH C8 column, 1.7 μm 2.1×50 mm. The proteins were analyzed using the following gradient: from 5% to 60% B for 4 min. and from 60 to 100% B from 4 to 4.5 min.

TABLE 1
List of forward (f) and reverse (r) primers
used to generate PSD-95-PDZ2 mutants.
Mutations have been highlighted in
bold and underlined.

| | | | SEQ |
|---|---|---|---|
| | | | ID |
| Mutation | | | NO |
| K165A | Primer (f): | CTGATCGCGGGCCCGAA | 437 |
| | Primer (r): | TTTGATTTCCATCACTTT | 438 |
| | | TTCCGC | |
| K168A | Primer (f): | CCCGGCGGGCCTGGGC | 439 |
| | Primer (r): | CCTTTGATCAGTTTGATTTCC | 440 |
| K193A | Primer (f): | GTGACCGCGATTATTGAAGGCG | 441 |
| | Primer (r): | ATAAATGCTGTTATCGCCCGG | 442 |
| H225A | Primer (f): | GATGTGATGGCGGAAGATGC | 443 |
| | Primer (r): | TTCCAGGCCCACGCTGTTC | 444 |
| K233A | Primer (f): | GCGCTGGCGAACACCTATGA | 445 |
| | Primer (r): | CGCCACCGCATCTTCATGCA | 446 |
| F172I | Primer (f): | CTGGGCATTAGCATTGCGGG | 447 |
| | Primer (r): | GCCTTTCGGGCCTTTGATCAG | 448 |
| S173A | Primer (f): | GCTTTGCGATTGCGGGCGGT | 449 |
| | Primer (r): | CCAGGCCTTTCGGGCCTTTGAT | 450 |
| T192A | Primer (f): | CATTTATGTGGCGAAAATTAT | 451 |
| | | TGAAGG-CGGTGC | |
| | Primer (r): | CTGTTATCGCCCGGAATATGC | 452 |
| | | TGGTTG | |
| V229A | Primer (f): | CATGAAGATGCGGCAGCGGCG | 453 |
| | Primer (r): | CATCACATCTTCCAGGCCCACGC | 454 |
| N180A | Primer (f): | GGTGTGGGCGCGCAGCATATTC | 455 |
| | Primer (r): | GCCCGCAATGCTAAAGCCCAGGC | 456 |
| E226A | Primer (f): | GTGATGCATGCGGATGCGGTG | 457 |
| | Primer (r): | ATCTTCCAGGCCCACGCTGTTC | 458 |
| F172A | Primer (f): | CTGGGCGCGAGCATTGCGGG | 459 |
| | Primer (r): | GCCTTTCGGGCCTTTGATCAG | 460 |

Protein and peptide thiol labelling. The buffer of choice was PBS buffer, prepared by dissolving Gibco® PBS tablets in MQ H₂O·pH was adjusted to pH 6.7 using 0.1 M HCl. The buffer was degassed for 30 min. under N₂ flow. The protein or peptide was dissolved in 1 mL of degassed buffer and introduced in a 15 mL falcon with continuous stirring, closed with a septum and a constant flow of N₂. Meanwhile, 15 eq of tetramethylrhodamine-5-(and-6)C2 maleimide (TAMRA maleimide) dye were dissolved in 200 μL of DMSO and added to the to the mixture through the septum with a syringe. The reaction was left for 2 h protected from the light and with continuous stirring.

Labelled proteins were purified using desalted Sephadex G-25 in PD-10 (MWCO 3000 Da) desalting columns. Protein quality was assessed using an Agilent 6550 LC-MS Q-TOF. Protein total mass was then deconvoluted with the Mass Hunter software.

Labelled cyclic peptides were purified with a reverse phase column (Zorbax 300 SB-C18, 21.2 mm×250 mm) in a linear gradient of 5% to 45% B during 40 min., using a binary solvent system [A: H2O/TFA 99.9/0.1 and B: MeCN/TFA 99.9/0.1]. The final products were analyzed with a Waters Acquity UPLC system with a QDA mass detector module, using a linear gradient of 5 to 60% B over 6 min. [A: H₂O/TFA 99.9/0.1 and B: MeCN/TFA 99.9/0.1].

SPOT peptide array synthesis. To generate the cyclic nNOSβ-hairpin arrays, we used the Intavis MultiPep spotter (Intavis Bioanalytical instruments). The membranes employed in this assay were SynthoPlan APEG CE (standard amino-modified acid stable sellulose membrane with a PEG-spacer), 10×15 cm with a loading of 400 nmol cm⁻². Solutions of 0.3 M Fmoc-AA-OH and 0.3 M Oxyma Pure were prepared in N-methyl-2-pyrrolidone (NMP). The activator solution consisted of 0.3 M DIC and 0.3 M of 2,4,6-trimethylpyridine (Collidine) in NMP. The capping solution consisted of a solution of 1 M acetic anhydride and 0.05 M of 4-dimethylaminopyridine (DMAP) in NMP. The deprotection solution consisted of 20% piperidine in NMP. AA couplings were performed 1 h,4 times. Deprotection of the Fmoc groups was performed 15min,2 times. Membrane washing between couplings or deprotections was performed with NMP and ethanol. Elongation of peptides in cellulose membrane was started by coupling first the Fmoc-PEG(9)-OH (Iris Biotech) and the subsequent mixture of Fmoc-Gly-OH (25%) and Boc-Gly-OH (75%) to lower the total loading of the membrane. Afterwards, the resin was capped with the capping solution and extensively washed and soaked in bromophenol blue (BPB) as a quality control to reveal the SPOTS with a functional amino group. Then, we coupled the quasi-orthogonal group Fmoc-Glu-PP, and carried out the rest of the synthesis until peptide completion. The cellulose bound linear peptides were finally treated with a solution of 95% DCM, 3% TIPS and 2% TFA 3 times, 20 min. to remove the quasi-orthogonal group. Afterwards, the membrane was washed 5 times with DCM and neutralized with 5% DIPEA in DCM. The membrane was then washed 5 more times with DCM, 5 times with NMP and cyclized with 0.3M PyAOP and 0.3M of DIPEA in DMF for 3 h or overnight. Finally, the membrane was dried with DCM and the side-chain protecting groups were cleaved with the standard deprotection cocktail (95% TFA, 2.5% H₂O and 2.5% TIPS) or with reagent K [TFA 82.5%, phenol 5%, H₂O 5%, thioanisole 5% and 1,2-ethanedithiol (EDT) 2.5%] for 3 h.

SPOT membrane screening. Membranes were incubated with PBS pH 7.2+0.5% bovine serum albumin (BSA) for 1 h. After the incubation period, the membrane was dried and scanned with an Amershan Typhoon scanner, at 400V, Cy3 wavelength (532 nm). A TIF file of the screening and a file with the blank values were generated using the Image Quant software. Afterwards, a solution of 50 nM of TAMRA labelled PSD-95-PDZ2 domain was prepared in PBS pH 7.2+0.5% BSA and was added to the membrane. The membrane was left incubating in a Polymax 1040 rocking table for 1 h protected from the light. Passed the incubation time, the excess of labelled protein was removed by washing the membrane 3 times with PBS+0.5% BSA. Finally, the membrane fluorescence values were measured with the Amershan Typhoon scanner at 400V, Cy3 wavelength (532 nm). Using the Image Quant software, the TIF image of the screening and a file with the fluorescence values of the peptides with the TAMRA labelled PSD-95-PDZ2 were generated. The blank was then subtracted from the fluorescence values of the peptides screened against the TAMRA labelled PSD-95-PDZ2.

Fluorescence polarization (FP). FP assays were performed in a 384-well plate format. Fluorescence polarization was measured using a Safire 2 plate reader. The instrument Z-factor was optimized for each assay. The G-factor was calibrated to an initial milli polarization value of 20. The wavelength for the cyclic nNOS β-hairpin TAMRA probe was: ex: 530 nm and em: 580 nm. Every measurement was performed in NaPI 50 mM, 50 mM NaCl and 1% BSA at pH 7.2, 25° C. Fluorescence polarization saturation assays were performed by titrating 50 nM of cyclic nNOS β-hairpin TAMRA prove to a 1:1 dilution curve of the selected protein. The curves were done in triplicates. Then, the polarization was fitted into a one-site binding model using Prism software 8.0 (GraphPad), from which the K_{d} can be measured. Fluorescence polarization competition experiments were conducted by mixing a preformed protein/probe complex at fixed concentration (50 μM/50 nM) with varying unlabelled peptide concentrations ranging from 0.1-252 μM. The mili polarization (mP) values were plotted as a function of peptide concentration and fitted to a sigmoidal dose-response curve using Prism software 8.0 (GraphPad). The Kᵢ values were calculated according to Nikolovska-Coleska Z. et al.⁴⁰⁻⁴¹

Isothermal titration calorimetry (ITC). The ITC assays were performed in NaPi 50 nM, 50 nM NaCl, pH 7.2 buffer filtered with a Corning® bottle-top vacuum filter system of a pore size of 0.22 μM, that was used to dissolve the peptides. Proteins were dialyzed against this buffer using Amicon® Ultra-15 centrifugal filter units, with a MWCO 3000 Da.

This assay was performed on an ITC200. The instrument differential power (DP) was set to 10 and the syringe rotating speed to 600 RPM. The assay setup consisted of introducing the protein inside the cell and the peptide (titrant) on the syringe. Calorimetry was performed at 25° C. Each analysis was performed in triplicates. Furthermore, several runs of peptide into buffer were performed in order to remove injection residual heat. Runs were analyzed with Origin 7.0 software (OriginLab).

Crystallographic screenings. We co-incubated at 4° C. the PSD-95-PDZ2 protein with the cyclic nNOS β-hairpin peptide (WT) and the disubstituted variant (T112W T116E). The concentration of PSD-95-PDZ2 was always set to 15 mg mL⁻¹ while we screened different ratios of cyclic peptide (1.5, 5 or 10 eq). The screenings were performed by the sitting drop method using 96-well COC Protein Crystallization Microplate with 3:1, 1 μL Conical Flat Bottom. Formed crystals from the screening were soaked in polyethylene glycol 400 (PEG400) before flash-freezing in liquid nitrogen. Diffraction data was collected at the Swiss Light Source, beamline SLS PX X₀₆₅ and was processed with Aimless (CCP4 suite).⁴² Molecular replacement and structure refinement was solved by using PHASER (Phenix software).⁴³ The search model was prepared from the crystal structure of nNOS-PDZ/Syntrophin-1-PDZ (PDB:1QAV)⁴⁴ using the Chimera software (USCF Chimera).⁴⁵ Crystal structures were validated in the PDB OneDep validation server.

Circular dichroism (CD). All CD spectra was collected using Jasco J-1500 Circular Dichroism Spectrophotometer using 1-mm pathlength quartz cuvettes. Protein samples were around 20 μM concentration in 50 mM NaPi, 50 mM NaCl pH 7.2 buffer. Data was collected in millidegrees of ellipticity, and then converted to mean residue ellipticity.

In silico unnatural amino acid (UAA) selections. To dock the cyclic nNOS β-hairpin in the binding pocket of PSD-95-PDZ2, the complex of nNOS/Syntrophin-1 (PDB: 1QAV)⁴⁴ was superimposed to the structure of PSD-95-PDZ1-2 (PDB: 3GLS).⁴⁶ The two PDZ domains of PDS-95-PDZ-2 and Syntrophin were swapped. The nNOS peptide was cyclized with the 3D builder tools in Maestro 2018-4 and residues involved in cyclization were minimized. 1 μs molecular dynamics simulation at 300 K were carried out using Desmond 5.6 with the OPLS3e force field in conjunction with TIP3P water model. First, 200 ns of the trajectory were used as equilibration and discarded. The frames from the remaining 800 ns were clustered using gromos clustering in GROMACS 2018-3 with an RMSD cut-off of 0.08 nm. Representative structures of the 3 largest clusters were selected for the UAA scan.

The UAA scan was performed on the selected structures using the residue scanning protocol in Maestro with a 4.5 Å refinement distance cut-off and side-chain prediction with backbone minimization. We used a library containing all available Fmoc protected amino acids in MolPort (Mar. 1, 2019) where the stereochemistry of the C^{a} was specified. To determine protonation state, we used LigPrep at pH 7.4+/−2.0 before adding the residues. Final size of the library contained 542 different amino acids. Hits from the UAA scan with ΔΔG_{affinity} values<−5 kJ mol⁻¹ K⁻¹ and ΔΔG_{stability}<0 kJ mol⁻¹ K⁻¹ in at least 2 of the 3 representative structures were selected based on visual inspection.

Labelling of Dynabeads™ M-270 Amine with peptides. Peptides were coupled to Dynabeads™ M-270 Amine by a thioether bond. Dynabeads™ (10 μL of suspension per pull-down experiment) were washed with DMF (2×1 mL) in 1.5 mL safe-lock tubes. Afterwards, the Dynabeads™ were incubated for 1 h at room temperature with 0.1 M bromoacetic anhydride with 5% DIPEA in DMF. The Dynabeads™ were washed with DMF (2×1 mL) and incubated with the solubilized peptide in DMF (120 μg of peptide per 200 μL of Dynabeads™ suspension in 1 mL DMF) for 3 h at room temperature. The supernatant was removed, and the beads were washed with DMF (2×1 mL) before incubation with 0.1 M β-mercaptoethanol with 5% DIPEA in DMF (1 mL) for 1 h at room temperature. Afterwards, the supernatant was removed, and the beads were washed with DMF (2 times, 1 mL) and PBS buffer, pH 7.4 (3 times, 1 mL). The labelled peptide-Dynabeads™ were stored in PBS buffer (10 μL of PBS buffer for 10 μL of starting suspension) at 4° C.

Lysis of whole mouse brains. Adult mouse brains (avg. mass: 0.4 g) were homogenized with a 15 mL tissue grinder in homogenization buffer on ice [10 mM NaCl, HEPES (pH 7.3), 320 mM Sucrose with Complete™ EDTA-free protease inhibitor and PhosSTOP™ phosphatase inhibitor; 1 mL per brain]. The homogenate was centrifuged at 1000 g (MULTIFUGE 3 L-R) for 10 min. at 4° C. and the supernatant was transferred to a new tube. The supernatant was centrifuged at 18500 g for 45 min. at 4° C. The supernatant S-Frac (Note: “S-Frac” contains cytosolic proteins) was collected. Concentration was measured, diluted to a concentration of 2 mg mL⁻¹, aliquoted to 1 mL and stored at −80° C. The pellet was resuspended in 1 mL per 1 mL of removed supernatant, 50% homogenization buffer and 50% detergent buffer [100 mM NaCl, 50 mM Tris-Cl (pH 8), 2% (w/v) sodium desoxycholate] and incubated for 1 h at 4° C. The insoluble proteins were removed by centrifugation at 20817 g (Eppendorf Centrifuge 5427 R) at 4° C. for 45 min. The supernatant M-Frac (Note: “M-Frac” contains membrane-bound and transmembrane proteins as well as membrane-bound protein complexes) was collected and its concentration was measured, diluted to a concentration 2 mg mL⁻¹, aliquoted to 1 mL and stored at −80° C.

Affinity purification. 20 μg of peptide-labelled Dynabeads™ was added to 500 μL of PBS buffer. The supernatant was removed and incubated with 1 mL of 2 mg mL⁻¹ brain lysate (S-Frac or M-Frac) for 2 h at 4 to 10° C. The supernatant was removed and washed with washing buffer I [50 mM Tris-HCl (pH 7.4), 150 mM NaCl and 1% (v/v) Triton X-100 in H₂O; 2 times, 1 mL] and washing buffer II [50 mM Tris-HCl (pH 7.4), 150 mM NaCl and 0.1% (v/v) Triton X-100 in H₂O; 2 times, 1 mL]. Then, it was resuspended with 20 μL of 2×Laemmli Buffer to the tubes and incubated for 10 min. at 95° C.

Analysis of pull-down experiment by MS. The pull down/2×Laemmli buffer mixture was loaded on Invitrogen NuPAGE® 4-12% Bis-Tris Gel (1.0 mm×12 well). Afterwards, an SDS gel was run in lnvitrogen™ MOPS Buffer and in an lnvitrogen™ Novex Mini-Cell at 80 V until the sample entered the gel (approx. 5 min.). The gel was then washed with H₂O (50 mL) for 5 min. and subsequently incubated with Imperial™ Protein Stain (50 mL) overnight. The gel was washed with H₂O (3 times, 50 mL for 30 min. each). The bands were extracted and transferred separately into a 96 well plate. Two initial washes were performed by incubation with 0.1 M ammonium bicarbonate in H₂O/MeCN (50:50, 100 μL well⁻¹) for 10 min. each. Afterwards, the gels were first incubated with 0.1 M ammonium bicarbonate in H₂O (50 μL well⁻¹). After 5 min., MeCN (50 μL per well) was added and left incubating for 15 min. more. This washing procedure was repeated until the Imperial™ staining disappeared. Then, the gels were incubated with 10 mM DTT and dissolved in 0.1 M ammonium bicarbonate in H₂O (100 μL per well) and incubated at 56° C. for 45 min. on an Echoterm™ heating plate. After that time, the solution was replaced rapidly with 55 mM iodoacetamide dissolved in 0.25 M ammonium bicarbonate in H₂O (100 μL per well) and incubated for 30 min. protected from light. Afterwards, the gels were washed with 0.25 M ammonium bicarbonate in H₂O/MeCN (50:50, 100 μL per well) for 5 min. each. Later on, 12.5 ng of modified Trypsin, Porcine in 0.25 M ammonium bicarbonate in H₂O (75 μL per well; 12.5 ng μL⁻¹) was added. Then, the plate was firstly incubated for 15 min. on ice and subsequently incubated over night at 37° C. To perform the digestion, 5% of formic acid in H₂O (75 μL well⁻¹) was added and left incubating for 5 min. in a Bransonic™ ultrasonic bath. The supernatant was transferred to 0.5 mL safe-lock tubes. Afterwards, the gels were extracted two times with 5% formic acid in H₂O/MeCN (40:60, 75 μL per well). The combined fractions were lyophilized. The sample was resolubilized in 0.1% TFA 4% MeCN and analyzed by an UltiMate™ 3000 UHPLC system equipped with an Orbitrap Fusion™ Lumos™ mass spectrometer, a precolumn PepMap™ 100 (100 μm×2 cm, nanoViper, C18,5 μm, 100 Å) and a column PepMap™ RSLC C18 (2 μm, 100 Å, 75 μm×50 cm, 37° C.). The separation method was based on a binary buffer system [A: TFA/MeCN/H₂O, 0.5/2/97.5 and B: formic acid/MeCN/H₂O, 0.1/20/79.9] in a flow rate of 0.3 mL min⁻¹. Peptides were eluted from the analytical column by a two-steps linear gradient: 4-25% MeCN/H₂O; 0.1% formic acid for 40 min. and 25-50% MeCN/H₂O; 0.1% formic acid for 10 min.

### Example 2

### Development of a Cyclic nNOS β-Hairpin Mimic Peptide

First, a cyclic nNOS β-hairpin peptide, cyclo-(C105THLETTFTGDGTPKTIRVTQ¹²⁴pG) (SEQ ID NO: 428), was synthesized using native chemical ligation (NCL) as described above. Then, the peptide was labelled with TAMRA maleimide using the free thiol group from the cysteine. The cyclized, fluorophore conjugated peptide was then tested in FP saturation assays as described above against the three recombinantly expressed PSD-95-PDZ1, 2 and -3 domains. The results indicated that the cyclic nNOS β-hairpin mimic peptide binds to PSD-95-PDZ1 and 2 with a K_{d} of 3.4±0.3 μM and 1.0±0.1 μM, respectively, while exhibiting only weak binding affinity to PSD-95-PDZ3 (K_{d}=52.0±10.5 μM).

Hence, the cyclic peptide cyclo-(CTHLETTFTGDGTPKTIRVTQpG) (SEQ ID NO: 428), wherein the sequence of THLETTFTGDGTPKTIRVTQ (SEQ ID NO: 429) corresponds to positions 105 to 124 of native nNOS, labelled with TAMRA binds to PSD-95-PDZ1 and 2.

### Example 3

### Cyclic nNOS β-Hairpin Mimic Peptide has Superior Affinity for PSD-95-PDZ2 Compared to Linear Peptide

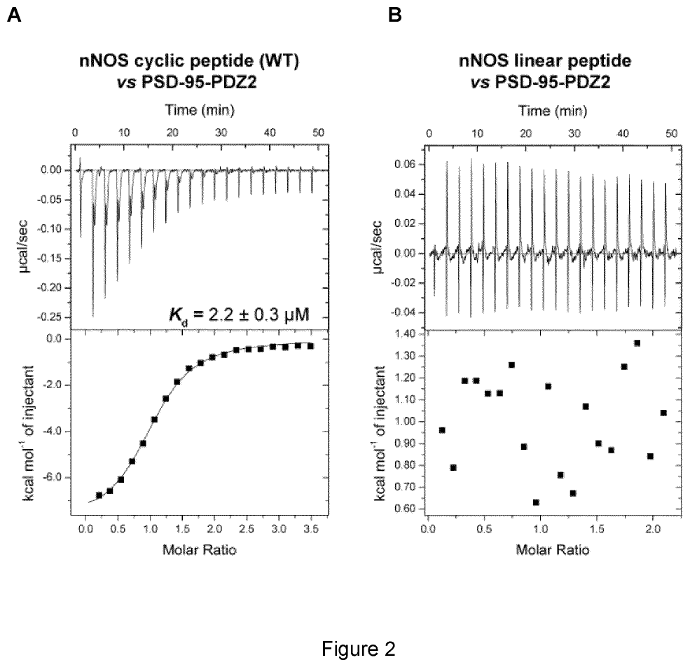

To explore if the cyclization of the nNOS β-hairpin mimic peptide is necessary for retaining the binding affinity to PDZ2, a linear version of nNOS peptide with a free C terminal was synthesized. ITC experiments were the conducted as described above with both the cyclic and linear nNOS peptide analogues (FIG. 2). The results showed that the cyclic nNOS β-hairpin peptide was binding to PSD-95-PDZ2 displaying affinity of 2.2±0.3 μM which was the same range as measure by FP. In contrast, the linear peptide did not show binding at any tested concentrations (up to 100 μM).

### Example 4

### Various Strategies for Cyclization of nNOS β-Hairpin Mimic Peptide

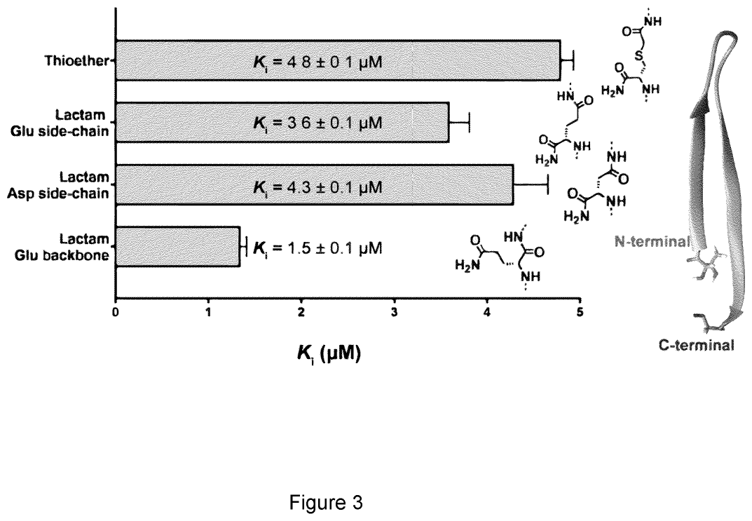

Four different strategies for cyclization of the nNOS β-hairpin mimic peptide were investigated, including employing a side chain like the thioether bridged analogue, a lactam Glu side chain, a lactam Asp side chain and a Glu backbone cyclized peptide. The cyclic nNOS β-hairpin variants of the nNOS β-hairpin mimic peptides were synthesized on resin and evaluated using FP competition assay as described above. Interestingly, three of the tested cyclization strategies resulted in slightly decreased binding affinities to the PSD-95-PDZ2 domain, see Table E1 and FIG. 3.

TABLE E1
Ki values, SEM and purity of different cyclic nNOS β-hairpin mimic scaffolds
measured against the recombinantly expressed PSD-95-PDZ2 and the cyclic
nNOS β-hairpin mimic TAMRA probe. * Peptides were synthesized on Rink Amide
ChemMatrix® resin, thus, the quasi-orthogonal cyclization building block (C, E or D) is
modified after cleavage. For simplicity, unmodified starting amino acid has been kept.
Purity: >95%.

| SEQ | | | | |
|---|---|---|---|---|
| ID | | | Kᵢ | |
| NO: | Peptide sequence | Type | (uM) | SEM |
| 428 | Cyclo-(C¹⁰⁵THLETTFTGDGTPKTIRVTQ¹²⁴pG) | NCL | 1.01 | 0.07 |
| 430 | Cyclo-(Ac¹⁰⁵THLETTFTGDGTPKTIRVTQ¹²⁴pGC)* | Thioether | 4.80 | 0.08 |
| 431 | Cyclo-(¹⁰⁵THLETTFTGDGTPKTIRVTQ¹²⁴pG(Eγ))* | Glu side- | 3.60 | 0.12 |
| | | chain | | |
| | | lactam | | |
| 432 | Cyclo-(¹⁰⁵THLETTFTGDGTPKTIRVTQ¹²⁴pG(Dγ))* | Asp side- | 4.29 | 0.21 |
| | | cahin | | |
| | | lactam | | |
| 433 | Cyclo-(¹⁰⁵THLETTFTGDGTPKTIRVTQ¹²⁴pG(Eα))* | Glu | 1.50 | 0.04 |
| | | backbone | | |
| | | lactam | | |

The strategies employing a side chain like the thioether bridged analogue, the lactam Glu side chain and the Asp side chain all reduced peptide binding affinity by 2- to-3-fold when compared to the NCL peptide. The Glu backbone cyclized peptide exhibited binding affinity in the same range as the NCL peptide (Kᵢ=1.5±0.2 μM).

### Example 5

### Deep Mutational Scan of the β-Hairpin Region in SPOT Arrays

The importance of every AA of the nNOS β-hairpin region (¹⁰⁵THLETTFTGDGTPKTIRVTQ¹²⁴, SEQ ID NO: 429) was probed using SPOT peptide arrays.

Based on the obtained results in Example 4, the lactam Glu backbone was chosen as the wild type (WT) scaffold. The resin used was Rink Amide CM resin, and hence, the Glu residue that used for cyclization was converted to a Gln after cleavage from the resin. The initial AA has been left on the sequences (Eα) for simplification.

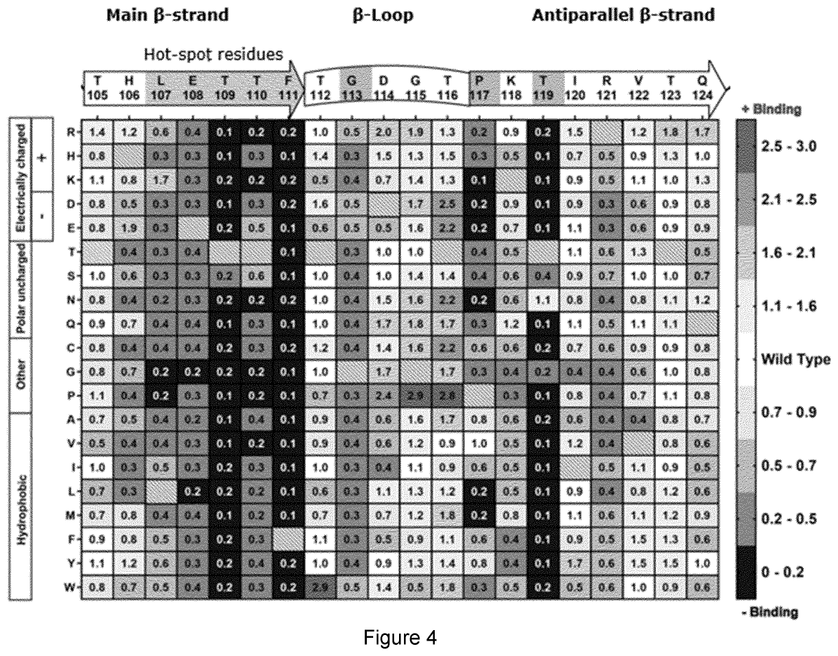

A deep mutational scan was performed on the WT scaffold, exchanging each AA to the remaining 19 L-AAs. Each array contained three copies of the individual peptides as technical replicates. Additionally, three controls were included in the design: the cyclic nNOS β-hairpin WT control (positive control), a linear nNOS peptide (cyclization control) and a cyclic nNOS β-hairpin with a F111V substitution (negative control previously tested in FP competition assays). For array screening, a mutation in PSD-95-PDZ2 residue V178 (PSD-95-PDZ2-V178C) was introduced, which was subsequently labelled with TAMRA maleimide. The synthesized peptide arrays were then screened with the TAMRA labelled PSD-95-PDZ2-V178C domain, and the resulting fluorescence intensities of the individual peptides were normalized to the WT peptide values. The final data are represented as a heat map of normalized fluorescence intensities of the peptide variants from the deep mutational scan (FIG. 4).

Based on the SPOT-obtained fluorescence intensity, the 57 most promising cyclic nNOS β-hairpin peptide analogs were re-synthesized and characterized (Table 2 below).

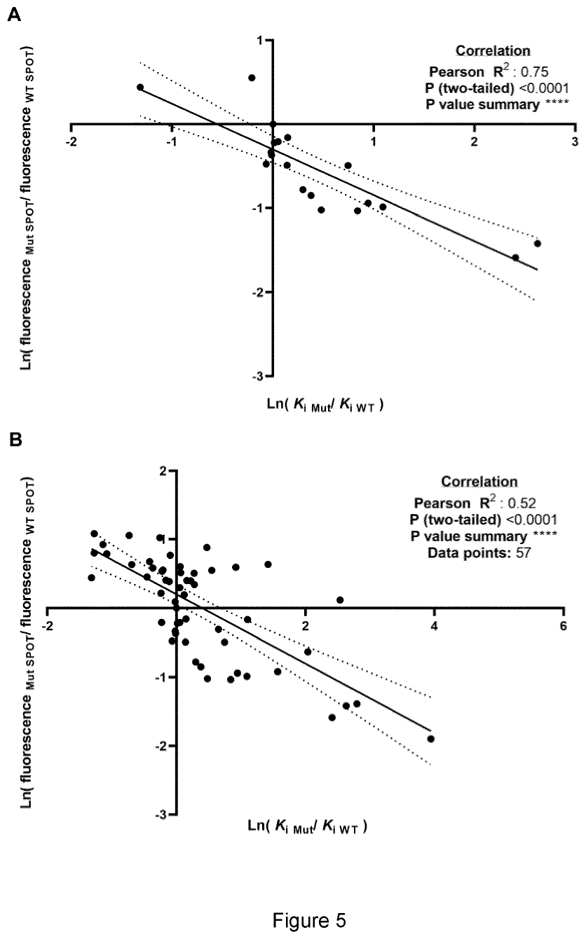

The FP-derived K values of the individual peptides were normalized to WT peptide K data (FIG. 5A-B). Subsequently, this data was correlated to the normalized SPOT fluorescence values. FP binding and SPOT fluorescence values correlated well for the alanine scan with a 75% Pearson R². However, when including all tested different substitutions (57 peptides) representing distinct modifications at different positions the Pearson R² dropped to 52%. The observed drop may be explained by differences in the cyclization yield when cyclizing obtained on the support. For example, nature of the introduced AA side chain (bulkiness, positively/negatively charged . . . ), bulkiness of the protecting group of the incorporated AA and/or mutation position in the hairpin structure (e.g. in the loop region), might influence cyclization yields. Since the SPOT array results is based on screening and fluorescence intensity of crude peptides, unforeseen impurities/truncations might lead to false positives or negatives (mutations with higher/lower cyclization yield than WT). Therefore, when comparing the SPOT-derived binding data with the Kᵢ values of purified peptides some discrepancies are expected.

TABLE 2
FP competition Kᵢ values, SEM, relative Kᵢ normalized
SPOT fluorescence and purity of the substituted cyclic
nNOS β-hairpin peptides selected from the positional
scan. FP inhibition constants and SPOT fluorescence
counts are normalized to the respective WT value.

| | | | | | Normalized | |
|---|---|---|---|---|---|---|
| SEQ | | | | Relative | SPOT | |
| ID | | Kᵢ | | Kᵢ | fluorescence | Purity |
| NO | Mutation | (μM) | SEM | (K_{i mut}/K_{i WT}) | (Mut/WT) | % |
| 18 | T112W | 0.72 | 0.02 | 0.48 | 2.74 | >95 |
| 19 | T112D | 0.95 | 0.09 | 0.63 | 1.03 | >95 |
| 20 | T122H | 1.98 | 0.08 | 1.32 | 1.49 | >95 |
| 21 | F111V | 77.40 | 4.80 | 51.60 | 0.05 | >95 |
| 22 | L107K | 1.20 | 0.10 | 0.80 | 0.77 | >95 |
| 23 | H106E | 6.19 | 0.09 | 4.13 | 0.87 | >95 |
| 24 | T105Y | 1.47 | 0.06 | 0.98 | 1.32 | >95 |
| 25 | T105G | 4.50 | 0.06 | 3.00 | 1.17 | >95 |
| 26 | H106Y | 1.18 | 0.05 | 0.79 | 1.47 | >95 |
| 27 | H106F | 1.19 | 0.04 | 0.79 | 1.17 | >95 |
| 28 | D114K | 2.87 | 0.05 | 1.91 | 2.62 | >95 |
| 29 | D114P | 2.40 | 0.05 | 1.60 | 5.28 | >95 |
| 30 | G115P | 0.42 | 0.04 | 0.28 | 2.33 | >95 |
| 31 | T116P | 1.16 | 0.01 | 0.77 | 2.60 | >95 |
| 32 | T116D | 0.48 | 0.02 | 0.32 | 1.85 | >95 |
| 33 | T116E | 0.42 | 0.01 | 0.28 | 1.37 | >95 |
| 34 | D114R | 0.99 | 0.03 | 0.66 | 5.51 | >95 |
| 35 | G115R | 0.75 | 0.10 | 0.50 | 2.74 | >95 |
| 36 | T116W | 1.04 | 0.03 | 0.69 | 2.43 | >95 |
| 37 | T116M | 3.76 | 0.04 | 2.51 | 2.00 | >95 |
| 38 | T116C | 1.36 | 0.03 | 0.91 | 1.93 | >95 |
| 39 | T119N | 18.89 | 6.40 | 12.59 | 0.94 | >95 |
| 40 | T116N | 0.51 | 0.01 | 0.34 | 2.14 | >95 |
| 41 | I120R | 1.76 | 0.29 | 1.17 | 2.00 | >95 |
| 42 | I120Y | 1.60 | 0.26 | 1.07 | 2.02 | >95 |
| 43 | V122R | 1.69 | 0.13 | 1.13 | 1.43 | >95 |
| 44 | T123R | 1.59 | 0.03 | 1.06 | 2.30 | >95 |
| 45 | Q124R | 1.97 | 0.09 | 1.31 | 3.50 | >95 |
| 46 | V122F | 1.34 | 0.08 | 0.89 | 1.81 | >95 |
| 47 | V122Y | 1.27 | 0.05 | 0.85 | 1.48 | >95 |
| 48 | T123F | 1.58 | 0.08 | 1.05 | 1.61 | >95 |
| 49 | T123Y | 1.87 | 0.06 | 1.25 | 1.65 | >95 |
| 50 | T119S | 7.19 | 0.32 | 4.79 | 0.34 | >95 |
| 51 | D114G | 2.59 | 0.17 | 1.73 | 2.54 | >95 |
| 52 | T109S | 24.51 | 7.89 | 2.51 | 2.00 | >95 |
| 53 | T110E | 11.47 | 0.64 | 0.34 | 2.14 | >95 |
| 54 | T110D | 1.42 | 0.06 | 0.95 | 0.16 | >95 |

In conclusion, ¹⁰⁷LETTF¹¹¹ (SEQ ID NO: 434), G¹¹³, P¹¹⁷ and T¹¹⁹ were identified as hot-spot residues, and the T112W, G115A/P and T116E/D were identified as the most promising substitutions of the cyclic nNOS β-hairpin mimic peptide.

### Example 6

### Alanine Scanning and Mutational Study of Hot-Spot Residues for the Cyclic nNOS β-Hairpin Mimic Peptide

In order to validate the results from the deep mutational scan in Example 5 and to gain further insight into the non-canonical binding mechanism, an alanine scan was performed as well as additional selective substitutions on the most relevant residues identified from the scan. The peptides synthesizes and FP assay were conducted as described above.

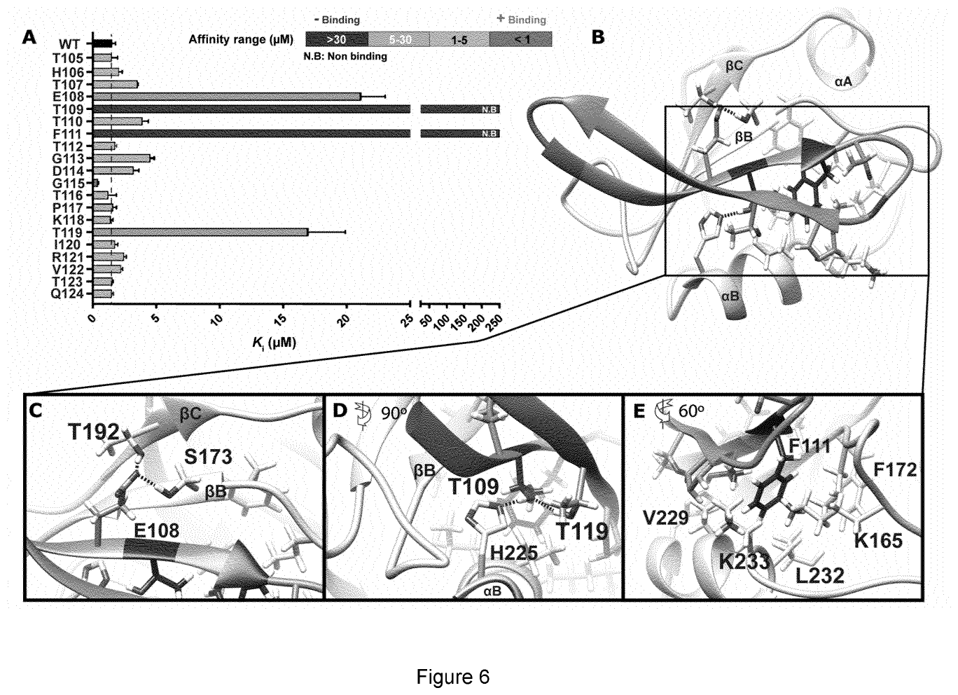

The FP (competition) results of the performed Ala scan (see Table 3 below) are aligned with the results from the deep positional scan in FIG. 6. It is demonstrated that E108, T109, F111 and T119 are the most important side chain interactions to bind to PSD-95-PDZ2. E108 is binding to AAs in the PSD-95-PDZ2 βB and βC such as T192 or S173 (FIG. 6C). T109 is part of the internal binding motif (-T-x-F-) and intermolecularly interacts through an H-bond with the side chain of T119 located in the antiparallel β-strand of the nNOS β-hairpin (FIG. 6D). This allows T109 to be on the right orientation to interact through its side chain with H225 in the PSD-95-PDZ2 aB and explains why T119A slightly decreases in affinity (Kᵢ=17.00±1.67 μM) while T109A abolishes binding of the cyclic nNOS β-hairpin peptide/PSD-95-PDZ2.

TABLE 3
FP competition Kᵢ values, SEM, relative Kᵢ normalized SPOT
fluorescence and purity of the Ala substituted cyclic nNOS
β-hairpin peptides. FP inhibition constants and SPOT fluorescence
counts are normalized to the respective WT value.

| | | | | | Normalized | |
|---|---|---|---|---|---|---|
| SEQ | | | | Relative | SPOT | |
| ID | | Kᵢ | | Kᵢ | fluorescence | Purity |
| NO | Mutation | (μM) | SEM | (K_{i mut}/K_{i WT}) | (Mut/WT) | % |
| 55 | T105A | 1.50 | 0.26 | 1.00 | 0.97 | >95 |
| 56 | H106A | 2.06 | 0.15 | 1.37 | 0.38 | >95 |
| 57 | L107A | 3.54 | 0.02 | 2.36 | 0.34 | >95 |
| 58 | E108A | 21.17 | 1.08 | 14.11 | 0.15 | >95 |
| 59 | T109A | N.B. | — | — | 0.05 | >95 |
| 60 | T110A | 3.93 | 0.26 | 2.62 | 0.66 | >95 |
| 61 | F111A | N.B. | — | — | 0.03 | >95 |
| 62 | T112A | 1.77 | 0.07 | 1.18 | 0.97 | >95 |
| 63 | G113A | 4.55 | 0.17 | 3.03 | 0.68 | >95 |
| 64 | D114A | 3.22 | 0.24 | 2.15 | 1.80 | >95 |
| 65 | G115A | 0.41 | 0.02 | 0.27 | 1.34 | >95 |
| 66 | T116A | 1.24 | 0.36 | 0.83 | 1.30 | >95 |
| 67 | P117A | 1.61 | 0.15 | 1.07 | 0.93 | >95 |
| 68 | K118A | 1.43 | 0.09 | 0.95 | 0.39 | >95 |
| 69 | T119A | 17.00 | 1.67 | 11.33 | 0.09 | >95 |
| 70 | I120A | 1.76 | 0.12 | 1.17 | 0.06 | >95 |
| 71 | R121A | 2.47 | 0.10 | 1.65 | 0.15 | >95 |
| 72 | V122A | 2.23 | 0.07 | 1.49 | 0.09 | >95 |
| 73 | T123A | 1.55 | 0.03 | 1.03 | 0.61 | >95 |
| 74 | Q124A | 1.51 | 0.06 | 1.01 | 0.42 | >95 |

Furthermore, the intramolecular T109-T119 interaction was explored by introducing several substitutions such as Ser, Asn or the Thr stereoisomer Allo-Thr. Ser and Asn substitutions in position T109 lowered the affinity of the cyclic peptide 7 and 19-fold respectively while Ser substitution in T119 lowered the affinity 25-fold. Interestingly, the Allo-Thr substitution in position T109 was able to abolish the interaction (Table 4 below). Finally, the side chain of F111 is facing inside the PSD-95-PDZ2 hydrophobic pocket formed by multiple residues (F172 in the carboxylate loop and V229 and L232 in the αB) (FIG. 6E).

TABLE 4
FP competition Kᵢ values, SEM and purity of the cyclic nNOS β-hairpin
peptide containing deletions or Allo-Thr substitutions.

| SEQ | | Kᵢ | | Purity |
|---|---|---|---|---|
| ID NO | Mutation or deletion | (μM) | SEM | % |
| 75 | ΔT112 | 0.69 | 0.05 | >95 |
| 76 | ΔT112, ΔG113, ΔD114, | N.B. | — | >95 |
| | ΔG115 | | | |
| 77 | ΔT116 | 1.43 | 0.06 | >95 |
| 78 | ΔD114 | 0.70 | 0.03 | >95 |
| 79 | ΔG113 | 0.86 | 0.09 | >95 |
| 80 | AlloT109 | N.B. | — | >95 |
| 81 | AlloT110 | 2.29 | 0.03 | >95 |
| 82 | AlloT105 | 2.65 | 0.13 | >95 |
| 83 | AlloT119 | 8.99 | 0.75 | >95 |

It is concluded that residues E108, T109, T111 and T119 are most important for the binding of the cyclic nNOS β-hairpin mimic peptide.

### Example 7

### N-Me Scan of the of the Cyclic nNOS β-Hairpin Mimic Peptide

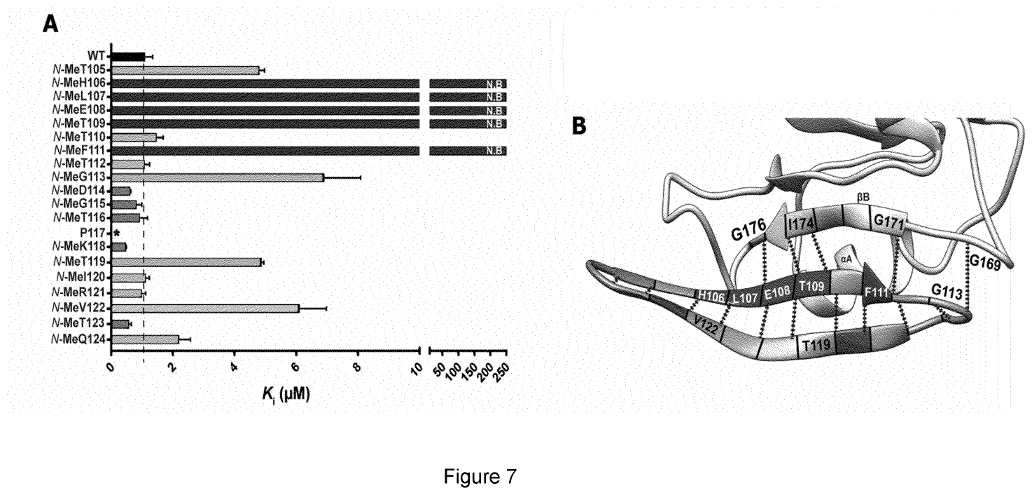

N-Me analogues of the cyclic nNOS β-hairpin mimic peptide were tested in FP competition assays as described above (FIG. 7A and Table 5 below). N-methylation of T105 (Kᵢ=4.8±0.1 μM) decreased the binding affinity by 5-fold, while N-Me variants of H106 to E108 abolished the interaction. This is likely because of inter-strand H-bonds stabilizing the peptide's hairpin fold (FIG. 7B).

TABLE 5
FP competition Kᵢ values, SEM and purity of the N—Me
scan of the cyclic nNOS β-hairpin peptide.

| SEQ | | Kᵢ | | Purity |
|---|---|---|---|---|
| ID NO | Mutation | (μM) | SEM | % |
| 84 | N—Me-T105 | 4.80 | 0.14 | >95 |
| 85 | N—Me-H106 | N.B. | 0.10 | >95 |
| 86 | N—Me-L107 | N.B. | — | >95 |
| 87 | N—Me-E108 | N.B. | — | >95 |
| 88 | N—Me-T109 | N.B. | — | >95 |
| 89 | N—Me-T110 | 1.47 | — | >95 |
| 90 | N—Me-F111 | N.B. | — | >95 |
| 91 | N—Me-T112 | 1.08 | — | >95 |
| 92 | N—Me-G113 | 6.19 | 0.09 | >95 |
| 93 | N—Me-D114 | 0.62 | 0.68 | >95 |
| 94 | N—Me-G115 | 0.82 | 0.70 | >95 |
| 95 | N—Me-T116 | 0.93 | 0.68 | >95 |
| 96 | N—Me-K118 | 0.48 | 0.14 | >95 |
| 97 | N—Me-T119 | 4.88 | 0.05 | >95 |
| 98 | N—Me-I120 | 1.12 | 0.04 | >95 |
| 99 | N—Me-R121 | 0.99 | 0.06 | >95 |
| 100 | N—Me-V122 | 6.17 | 0.07 | >95 |
| 101 | N—Me-T123 | 0.58 | 0.05 | >95 |
| 102 | N—Me-Q124 | 2.20 | 0.04 | >95 |

N-Me-T109 also abolished the interaction of the internal binding motif (-T-x-F-). N-Me-F111 also abolished the interaction as a relevant backbone H-bond with G171 on the βB of PSD-95-PDZ2 is removed. The impact of N-Me-G113 (Kᵢ=6.2±0.1 μM) binding affinity was less pronounced since H-bond might due to the steric hindrance of the methyl group on the Gly residue, which would reduce its flexibility. N-methylation of T119 (Kᵢ=4.9±0.1 μM) decreased the binding affinity potentially due to the steric hindrance introduced as T119 does not engage in a backbone H-bond (FIG. 7B). In contrast, N-Me-V122 (Kᵢ=6.2±0.1 μM) disrupting an inter-strand H-bond with H106, however, to a moderate extent. Interestingly, for some residues in the antiparallel β-strand of the cyclic nNOS β-hairpin peptide, N-methylation increased the binding affinity: N-Me-K118 (Kᵢ=0.5±0.1 μM) and N-Me-T123 (Kᵢ=0.6±0.1 μM) led to a 2-fold improved binding affinity.

### Example 8

### Alanine Scanning Mutagenesis of the PSD-95-PDZ2

In this study, the binding mode of the non-canonical β-hairpin peptide was compared with the canonical C-terminal tail of the ionotropic glutamate-type NMDAR subunit GluN2B (KLSSIESDV-COOH, SEQ ID NO: 435). Hence, a series of PSD-95-PDZ2 Ala mutants were expressed. Mutations were introduced at positions K165A, K168A, F172A, S173A, N180A, T192A, K193A, H225A, E226A, V229A and K233A.

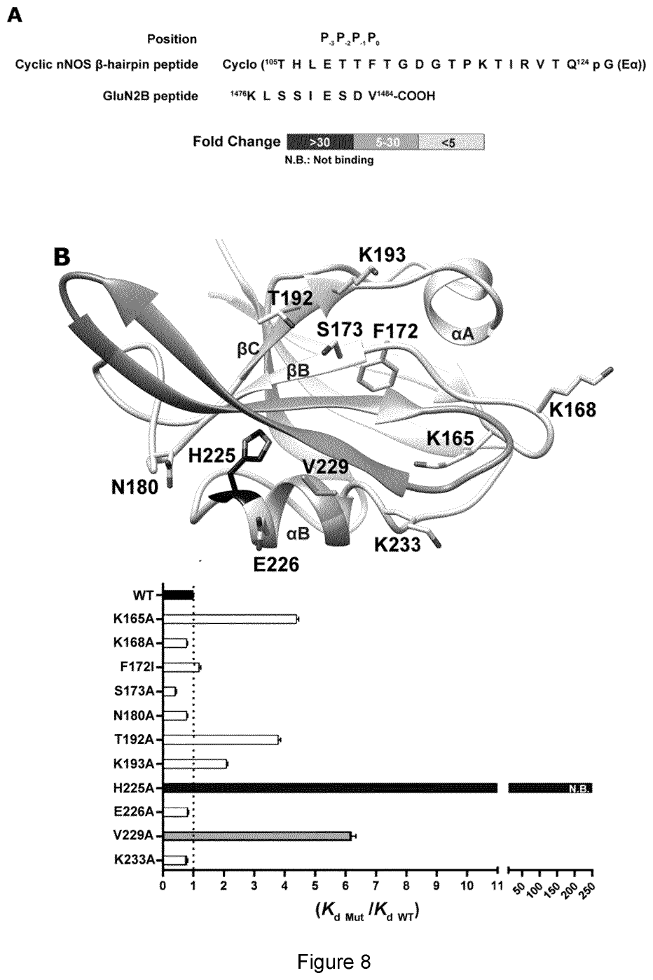

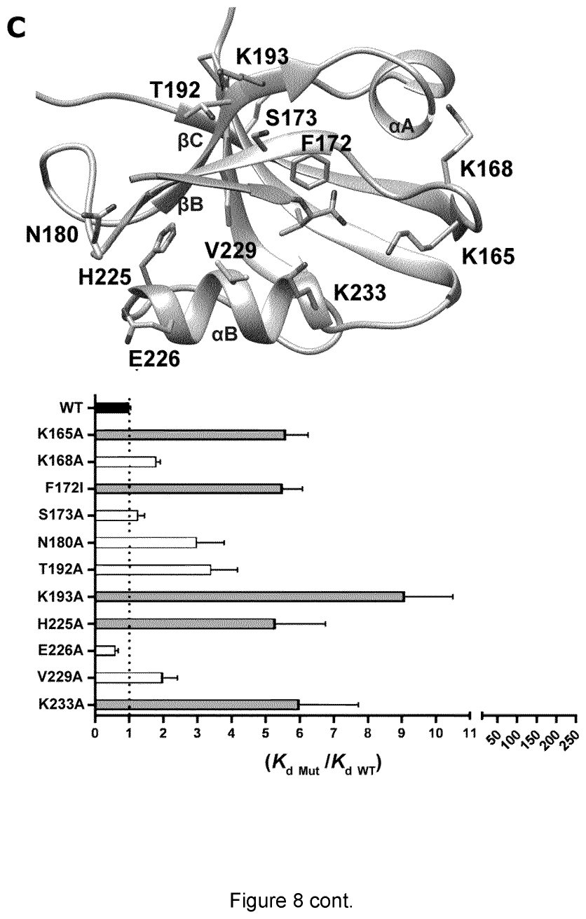

To compare the two binding modes, TAMRA labelled C-terminal GluN2B peptide and a TAMRA labelled cyclic nNOS β-hairpin mimic peptide were used as probes in FP saturation assays. Obtained binding affinities (K_{d} values) for each PSD-95-PDZ2 Ala mutants were normalized to PSD-95-PDZ2 WT values to obtain the fold change for each mutation (FIG. 8).

Interestingly, the cyclic peptide showed a completely different binding profile to the canonical GluN2B canonical peptide. For example, the H225A mutation completely abolished the cyclic nNOS β-hairpin interaction with PSD-95-PDZ2. In contrast, the canonical GluN2B peptide only showed a 5-fold decrease in affinity for the same mutation.

The V229A mutation also had a detrimental effect on the binding of the cyclic nNOS β-hairpin peptide (6-fold loss in affinity) in comparison to the GluN2B canonical peptide (2-fold).

The K165 mutation decreased binding affinity for the cyclic nNOS β-hairpin peptide (4-fold loss in affinity) as well as for the GluN2B canonical peptide (5-fold).

### Example 9

### Pull-Down Selectivity Comparison of Cyclic nNOS β-Hairpin Peptide to the C-Terminal Region of GluN2B

An affinity-based pull down assay was performed as described in Example 1 in order to compare the selectivity of the cyclic nNOS β-hairpin peptide to the C-terminal region of GluN2B (KLSSIESDV-COOH, SEQ ID NO: 435). Both compounds were immobilized on Dynabeads™ M-270 Amine beads and incubated with mouse (mus musculus) brain lysate. The lysate was separated in two fractions, membranal and cytosolic, by gradient centrifugation. The enriched proteins were analysed qualitatively and quantitatively by liquid chromatography coupled to tandem mass spectrometry (LC-MS/MS) after digestion with trypsin.

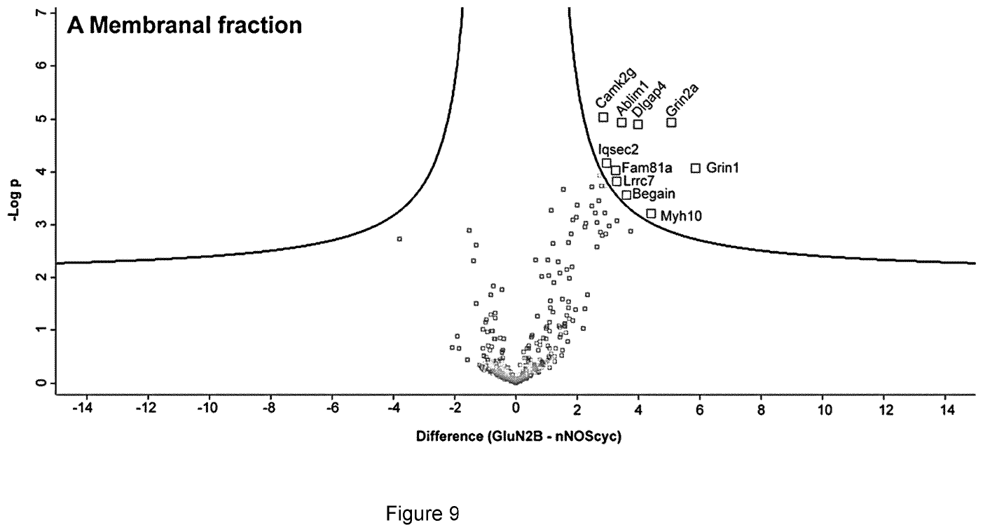

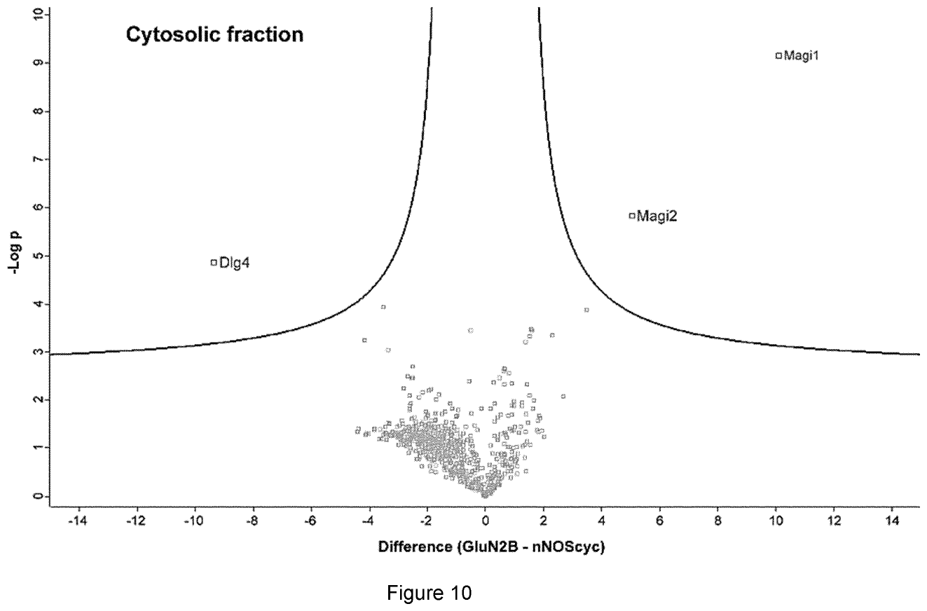

The results are visualized in Volcano plots (FIGS. 9 and 10), in which the proteins on negative X-axis are significantly enriched by the cyclic nNOS β-hairpin peptide over the canonical binding GluN2B peptide. In the membranal fraction (FIG. 9) several proteins got enriched by GluN2B, which themselves do not contain a PDZ domain. PDZ-containing proteins are not significantly enriched by either cyclic nNOS β-hairpin peptide or GluN2B.

In the cytosolic fraction (FIG. 10), in which PSD-95 is supposed to be present in a lower concentration due to the N-terminal palmitoylations, the linear peptide enriches significantly the multi-PDZ domain proteins membrane-associated guanylate kinase inverted MAGUK (Magi1 and Magi2). Interestingly, cyclic nNOS β-hairpin peptide significantly enriched PSD-95 (disc large homolog 4, DIg4), which could be due to very selective interaction to PSD-95, whereas the linear GluN2B peptide interacts non-selectively.

### Example 10

### Phosphorylation of the nNOS β-Hairpin Motif

The effect of phosphorylation is evaluated using fluorescent polarization competition experiments.

Materials and Methods

All Thr residues were individually phosphorylated, employing standard Fmoc SPPS methodology and using Fmoc-Thr(PO(OBzl)OH)-OH as a building block. Following SPPS, peptides were measured against recombinantly expressed PSD-95-PDZ2 and the cyclic nNOS TAMRA probe in fluorescent polarization competition experiments, as described in Example 1. Data was collected in triplicates and is presented as mean of Kᵢ values±SEM

Results

The cyclic nNOS β-hairpin mimic peptide were phosphorylated at positions corresponding to residues 105, 109, 110, 112, 116, 119 and 123 of the wild type nNOS. It was found that modifications made to residues 109, 110 and 119 caused disruptive effects, while modifications made to residue 116 caused a synergistic binding effect. An overview is found in Table 6 below.

TABLE 6
FP competition Kᵢ values, SEM and purity of the
phosphorylated cyclic nNOS β-hairpin analogues.

| SEQ | | Kᵢ | | Purity |
|---|---|---|---|---|
| ID NO | Mutation | (μM) | SEM | % |
| 103 | pT105 | 2.93 | 0.36 | >95 |
| 104 | pT109 | N.B. | — | >95 |
| 105 | pt110 | N.B. | — | >95 |
| 106 | pT112 | 2.68 | 0.01 | >95 |
| 107 | pT116 | 0.21 | 0.01 | >95 |
| 108 | pT119 | N.B. | — | >95 |
| 109 | pT123 | 1.15 | 0.04 | >95 |

These findings are in line with previous observations and modifications where T116D/E substituations also increased binding affinity to a similar extent, suggesting a preference for a negative charge in this position of the cyclic peptide.

### Example 11

### N-Methylations of a His Residue in the nNOS β-Hairpin Motif

The effect of N-methylation on His is evaluated using fluorescent polarization competition experiments.

Materials and Methods

The two possible imidazole nitrogens of His were pre-methylated prior to peptide synthesis employing standard Fmoc SPPS methodology. The starting building blocks were Fmoc-His(τ-Me)-OH [His(1-Me)] or Fmoc-His(π-Me)-OH [His(3-Me)]. Following SPPS, peptides were measured against recombinantly expressed PSD-95-PDZ2 and the cyclic nNOS TAMRA probe in fluorescent polarization competition experiments, as described in Example 1. Data was collected in triplicates and is presented as mean of Kᵢ values±SEM.

Results

The influence of methylation on the imidazole nitrogens of His were evaluated, and is presented in Table 7 below.

The results showed that methylation of N1 (N^{π}) slightly decreased the affinity (Kᵢ=2.9±0.1 μM) relative to the cyclic template whereas methylation of N3 (N^{τ}) led to a significant increase in affinity it with a Kᵢ of 0.40±0.05 μM.

TABLE 7
FP competition Kᵢ values, SEM and purity of the His side-
chain methylated cyclic nNOS β-hairpin analogues.

| SEQ | | Kᵢ | | Purity |
|---|---|---|---|---|
| ID NO | Mutation | (μM) | SEM | % |
| 110 | His(3-Me) | 0.40 | 0.01 | >95 |
| 111 | His(1-Me) | 2.90 | 0.10 | >95 |

A possible hypothesis is that the hydrogen in His N3 position has a polar nature and it is positioned facing a hydrophobic surface formed between L107 and V122 to H106. Methylation of this position would provide a stabilization effect of the hydrophobic surface and stabilize the peptide conformation, thus the increase in affinity.

### Example 12

### D-AA Scan of the nNOS β-Hairpin Mimic Peptide

The effect of D-amino acid substitution is evaluated using fluorescent polarization competition experiments.

Materials and Methods

L-amino acids were substituted for D-analogues (except for Gly) resulting in 18 analogues that were synthesized using commercially available Fmoc-D-AA-OH building blocks and standard Fmoc SPPS methodology. Following SPPS, peptides were measured against recombinantly expressed PSD-95-PDZ2 and the cyclic nNOS TAMRA probe in fluorescent polarization competition experiments, as described in Example 1. Data was collected in triplicates and is presented as mean of Kᵢ values±SEM.

Results

An overview of the results is presented in Table 8 below.

TABLE 8
Kᵢ values of the cyclic nNOS β-hairpin peptide
D-AA analogues measured against recombinantly expressed
PSD-95-PDZ2 and the cyclic nNOS TAMRA labelled
cyclic peptide in FP competition experiments.

| SEQ | | Kᵢ | | Purity |
|---|---|---|---|---|
| ID NO | Mutation | (μM) | SEM | % |
| 112 | T105t | 14.63 | 6.96 | >95 |
| 113 | H106h | 13.14 | 1.72 | >95 |
| 114 | L107l | N.B. | — | >95 |
| 115 | E108e | N.B. | — | >95 |
| 116 | T109t | N.B. | — | >95 |
| 117 | T110t | N.B. | — | >95 |
| 118 | F111f | N.B. | — | >95 |
| 119 | T112t | N.B. | — | >95 |
| 120 | D114d | 0.59 | 0.04 | >95 |
| 121 | T116t | 2.00 | 0.02 | >95 |
| 122 | P117p | 6.70 | 0.11 | >95 |
| 123 | K118k | 23.20 | 8.90 | >95 |
| 124 | T119t | N.B. | — | >95 |
| 125 | I120i | N.B. | — | >95 |
| 126 | R121r | 9.00 | 3.00 | >95 |
| 127 | V122v | N.B. | — | >95 |
| 128 | T123t | 1.83 | 0.07 | >95 |
| 129 | Q124q | 11.12 | 0.69 | >95 |

The results showed that introducing a D-AA in virtually any position of the cyclic peptide is detrimental for the binding affinity. The fact that a slight increase (Kᵢ=0.59±0.04 μM) in binding affinity is observed for the D114d substitution, might be explained by the fact that several Lys residues are found in the vicinity of D114, and D-Asp is slightly better positioned to bind one of these Lys residues.

### Example 13

### Development of High Affinity Cyclic Peptide Inhibitors of PSD-95-PDZ2

Following the successful single-point mutations presented in Examples 10-12, multiple mutations were analyzed for additive and/or synergistic effects.

Materials and Methods

Peptides were synthesized as previously described using standard SPPS methodology. Following SPPS, peptides were measured against recombinantly expressed PSD-95-PDZ2 and the cyclic nNOS TAMRA probe in fluorescent polarization competition experiments, as described in Example 1. Data was collected in triplicates and is presented as mean of Kᵢ values±SEM. An overview is presented in Table 9. Prospective cyclic peptide candidates were further analyzed using isothermal titration calorimetry (ITC) according to the procedure described in Example 1.

Results

Two highly potent cyclic peptides encompassing multi-point mutations were identified based on fluorescent polarization competition experiments, one being T112W, T116E substituted (Kᵢ=0.11±0.02 μM), the other being ΔT112, T116E substituted (Kᵢ=0.16±0.02 μM). These two were then further evaluated by isothermal titration calorimetry (ITC).

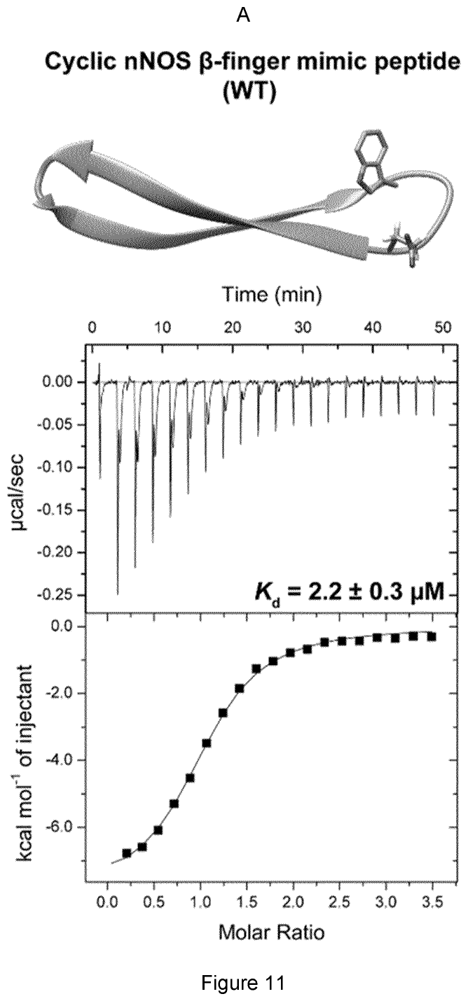

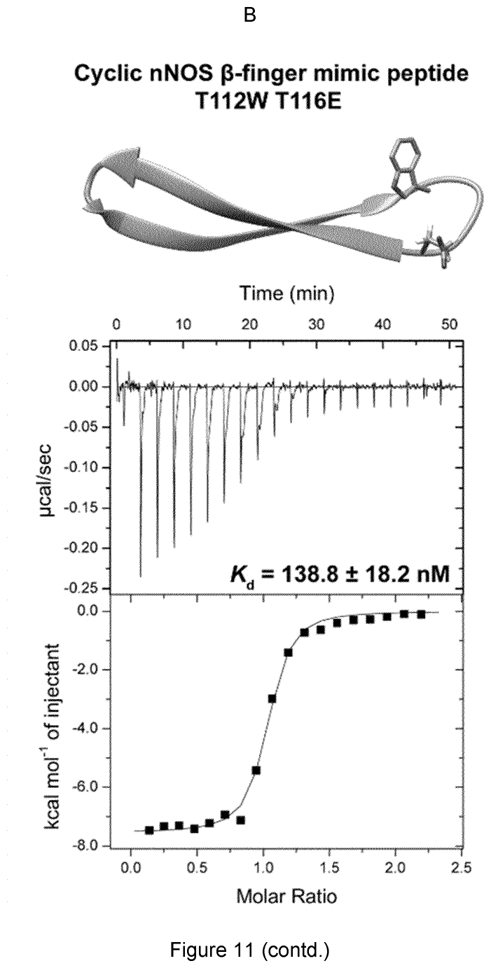

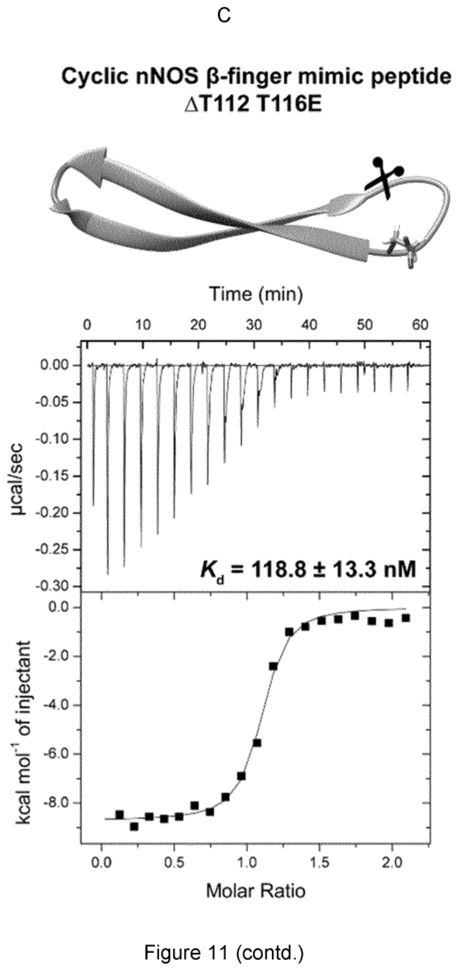

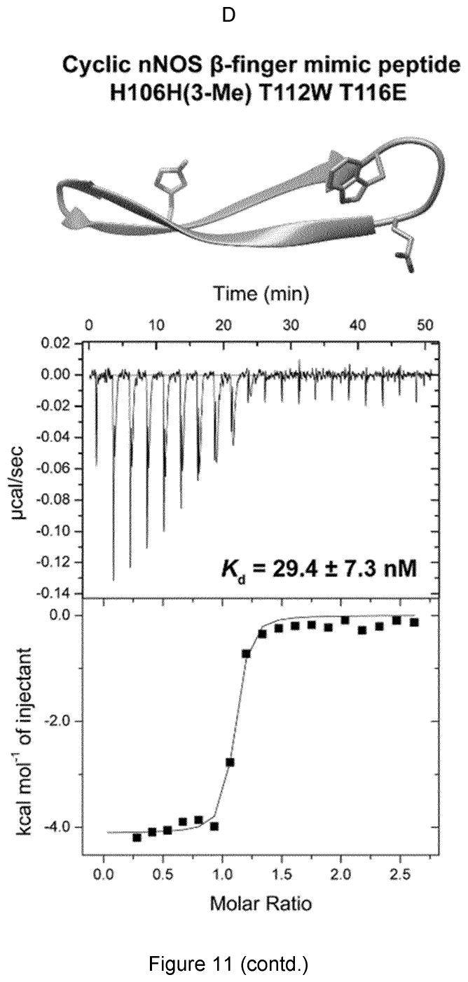

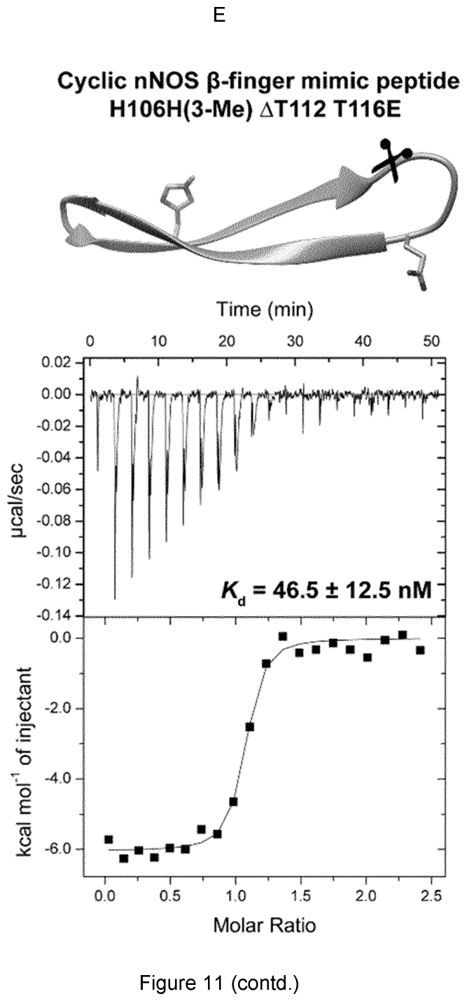

Compared to the wild-type cyclic nNOS mimc peptide (FIG. 2A), the ITC measurements (Table 10 below) aligned with fluorescent polarization assays and showed K_{d} values of 138.8±0.2 nM for T112W, T116E (FIG. 11B) and 118.8±13.1 nM for ΔT112, T116E (FIG. 110), thus showing 16- and 18-fold improved binding affinity, respectively compared to the wild-type peptide.

TABLE 9
FP competition Kᵢ values, SEM and purity of different
di and trisubstituted cyclic nNOS β-hairpin
peptide analogues for affinity enhancement.

| SEQ | | Kᵢ | | Purity |
|---|---|---|---|---|
| ID NO | Mutations | (μM) | SEM | % |
| 130 | T112W T116D | 0.65 | 0.02 | >95 |
| 14 | T112W T116E | 0.11 | 0.02 | >95 |
| 131 | T112W G115P | 0.95 | 0.02 | >95 |
| 132 | G115P T116E | 22.52 | 3.67 | >95 |
| 15 | ΔT112 T116E | 0.16 | 0.06 | >95 |
| 133 | T112W G115P | N.B. | — | >95 |
| | T116E | | | |
| 134 | T112W G115A | 0.26 | 0.04 | >95 |
| | T116E | | | |
| 135 | T112W D114d | 0.19 | 0.02 | >95 |
| | T116E | | | |
| 136 | ΔT112 D114d | 0.36 | 0.07 | >95 |
| | T116E | | | |

Additional substitutions introducing His(3-Me) into either of these two peptide candidates, showed an additive effect resulting in further improvement on binding affinities (Table 10), thus the cyclic peptide with three substitutions, H106H(3-Me), T112W and T116E, showed a remarkably improved affinity with a K_{d} value of 29.4±7.3 nM (FIG. 11D), thus being 75-fold more potent than the parent scaffold. Similarly, the same scaffold comprising the two mutations and a deletion, H106H(3-Me), ΔT112 and T116E had a K_{d}=46.5±12.5 nM (FIG. 11E) and a 47-fold increase in affinity relative to the wild-type peptide.

TABLE 10
ITC K_{d} values and thermodynamic signatures of the cyclic nNOS β-hairpin peptide
(WT) and the best di and trisubstituted variants at (25° C.) with PSD-95-PDZ2.

| | | | | | −ΔG = ΔH − |
|---|---|---|---|---|---|
| SEQ | | K_{d} | ΔH | −ΔS*T | ΔS*T |
| ID NO | Mutation | (nM) | (cal mol⁻¹) | (cal mol⁻¹) | (cal mol⁻¹) |
| 12 | Cyclic WT | 2152.4 ± 268.9 | −7741.8 ± 683.7 | 349.9 ± 308.4 | −7479.3 ± 485.2 |
| 14 | T112W, T116E | 138.8 ± 18.2 | −7208.0 ± 838.1 | −2148.7 ± 759.0 | −9356.7 ± 80.0 |
| 15 | ΔT112, T116E | 118.2 ± 13.1 | −8524.5 ± 387.6 | −928.7 ± 420.4 | −9453.2 ± 64.4 |
| 16 | H106H(3-Me), | 29.4 ± 7.3 | −6637.8 ± 122.9 | −3659.8 ± 239.0 | −10297.5 ± 152.0 |
| | T112W, T116E | | | | |
| 17 | H106H(3-Me), | 46.5 ± 12.5 | −6108.3 ± 124.7 | −3915.7 ± 260.5 | −10024.0 ± 182.3 |
| | ΔT112, T116E | | | | |

### Example 14

### Non-Proteinogenic SPOT Array Screening of the Cyclic nNOS β-Hairpin Mimic Peptide

Non-proteinogenic amino acids were scanned for potential synergistic effect realized upon incorporation into the cyclic β-hairpin peptide sequence. Such effects can include increased binding affinity, improved stability and more.

Materials and Methods

Peptides comprising amino acids corresponding to residues 105-116 of the wild-type cyclic nNOS peptide were chosen as the scaffold for the study. An identical segment containing two mutations (T112W and T116E) were also included as an already optimized scaffold peptide. The 105-116 residues were chosen as the basis for the mutations as these were deemed most likely to impact increase in affinity. Peptides were synthesized in spot arrays as previously described in Example 1. The array was afterwards incubated with TAMRA-labelled PSD-95-PDZ2.

Results

The fluorescence data is presented below in detail in Tables 11 and 12. In summary, substitutions in F111 with halogenated analogues such as Phe-2-F, Phe-2-Cl, Phe-3-F and Phe-2-Br seems to increase the binding affinity, evidenced by a larger fluorescence output. For residue 106, positive aromatic residues such as PyA-4 or Phe-3-CH₂NH₂ increased the binding affinity. Methylation of H in residue 106 was also found to provide additive effects as previously described in Example 11. This trend was observed for both the wild-type and the optimized cyclic peptide scaffolds. For the wild-type scaffold, substitutions in residue 115 with Pro, Arg or mono- and dimethylated Arg also seemed to favour an increase in binding affinity.

TABLE 11
Fluorescence raw data (10⁶) and statistical parameters
of each non-proteogenic amino acid in the SPOT array
incubated with TAMRA labelled PSD-95-PDZ2.

| SEQ | | | | | | |
|---|---|---|---|---|---|---|
| ID | | Fluor. | Fluor. | Fluor. | | |
| NO | Mutation | Pep. 1 | Pep. 2 | Pep. 3 | Average | Std.dev |
| 12 | Cyclic WT | 3.41 | 3.68 | 3.24 | 3.44 | 0.2 |
| 137 | Linear WT | 0.84 | 2.16 | 1.58 | 1.52 | 0.7 |
| 138 | F111V | 0.28 | 0.62 | 0.91 | 0.60 | 0.3 |
| 139 | T116E | 7.57 | 8.45 | 7.85 | 10.70 | 1.9 |
| 140 | T116e | 2.07 | 4.31 | 3.91 | 14.87 | 4.3 |
| 141 | T116D | 8.23 | 7.12 | 8.88 | 7.96 | 0.4 |
| 142 | T116d | 3.65 | 4.01 | 4.63 | 3.43 | 1.2 |
| 143 | T116HomoSer | 5.02 | 4.37 | 4.89 | 8.08 | 0.9 |
| 144 | T116AlloThr | 5.08 | 4.01 | 4.88 | 4.10 | 0.5 |
| 145 | T116Glu(OAll) | 4.15 | 3.09 | 2.77 | 4.76 | 0.3 |
| 146 | T116Cys.Acid | 6.27 | 4.74 | 5.74 | 4.66 | 0.6 |
| 147 | T116Api | 5.13 | 4.66 | 3.64 | 3.34 | 0.7 |
| 148 | T116HomoGln | 6.25 | 4.05 | 5.07 | 5.58 | 0.8 |
| 149 | G115R(Me) | 7.93 | 4.03 | 4.37 | 4.48 | 0.8 |
| 150 | G115 R(Me)₂ | 6.11 | 4.50 | 4.75 | 5.12 | 1.1 |
| 151 | G115R | 10.31 | 5.93 | 6.50 | 5.44 | 2.2 |
| 152 | G115A | 4.49 | 3.67 | 4.66 | 5.12 | 0.9 |
| 153 | G115P | 10.19 | 5.48 | 8.95 | 7.58 | 2.4 |
| 154 | G115a | 2.54 | 1.84 | 2.35 | 4.28 | 0.5 |
| 155 | G115p | 1.79 | 2.15 | 2.41 | 8.21 | 2.4 |
| 156 | D114d | 4.08 | 2.80 | 3.42 | 2.25 | 0.4 |
| 157 | D114E | 1.66 | 2.18 | 2.27 | 2.12 | 0.3 |
| 158 | D114e | 4.13 | 3.07 | 3.81 | 3.43 | 0.6 |
| 159 | G113D-Cit | 2.49 | 2.64 | 1.98 | 2.04 | 0.3 |
| 160 | G113Cit | 1.60 | 1.62 | 1.61 | 3.67 | 0.5 |
| 161 | G113HomoCit | 1.53 | 1.84 | 1.53 | 2.37 | 0.3 |
| 162 | G113HomoGln | 3.74 | 5.56 | 4.18 | 1.61 | 0.0 |
| 163 | T112W | 6.40 | 5.19 | 4.47 | 1.63 | 0.2 |
| 164 | T112w | 3.01 | 1.70 | 1.24 | 4.49 | 1.0 |
| 165 | T112AlloThr | 1.28 | 2.24 | 2.11 | 5.35 | 1.0 |
| 166 | T112Phe-4-Me | 4.43 | 4.70 | 2.85 | 1.99 | 0.9 |
| 167 | T112D-Phe- | 2.05 | 1.13 | 1.53 | 1.88 | 0.5 |
| | (3,4Cl) | | | | | |
| 168 | T112Phe-4- | 3.27 | 5.12 | 3.23 | 4.00 | 1.0 |
| | NH₂ | | | | | |
| 169 | T112BIP | 4.30 | 3.34 | 3.43 | 1.57 | 0.5 |
| 170 | T112D- | 0.70 | 1.25 | 1.09 | 3.87 | 1.1 |
| | HomoCha | | | | | |
| 171 | T112HomoCha | 1.80 | 2.47 | 2.15 | 3.69 | 0.5 |
| 172 | T112Cha | 1.55 | 2.07 | 1.81 | 1.01 | 0.3 |
| 173 | T112D-Cha | 0.74 | 1.14 | 1.09 | 2.14 | 0.3 |
| 174 | T112Y | 2.07 | 3.05 | 4.29 | 1.81 | 0.3 |
| 175 | T112y | 1.20 | 1.65 | 1.28 | 0.99 | 0.2 |
| 176 | T112 D-Tyr-2- | 0.77 | 1.24 | 1.24 | 3.14 | 1.1 |
| | (Et)Cha | | | | | |
| 177 | T112D-Cit | 0.95 | 1.30 | 1.09 | 1.38 | 0.2 |
| 178 | T112Cit | 3.57 | 3.28 | 3.06 | 1.09 | 0.3 |
| 179 | T112D-Dap | 1.10 | 1.70 | 1.79 | 1.12 | 0.2 |
| 180 | T112Dap | 3.11 | 2.60 | 3.33 | 3.30 | 0.3 |
| 181 | T112Nal2 | 2.47 | 3.72 | 3.59 | 3.26 | 0.69 |
| 182 | T112IndaG | 3.09 | 2.20 | 2.62 | 2.64 | 3.09 |
| 183 | T112Trp-2-Me | 5.22 | 4.29 | 5.31 | 4.94 | 5.22 |
| 184 | T112Aic | 2.22 | 1.33 | 1.35 | 1.63 | 2.22 |
| 185 | T112F | 3.75 | 2.83 | 3.11 | 3.23 | 3.75 |
| 186 | T112f | 1.17 | 1.18 | 1.01 | 1.12 | 1.17 |
| 187 | T112H | 4.30 | 3.04 | 4.08 | 3.81 | 4.30 |
| 188 | T112HomoPhe | 2.56 | 2.00 | 1.71 | 2.09 | 2.56 |
| 189 | T112Naphtoic | 1.03 | 1.15 | 1.36 | 1.18 | 1.03 |
| | acid | | | | | |
| 190 | F111Cha | 0.74 | 0.83 | 0.78 | 0.78 | 0.74 |
| 191 | F111-D-Cha | 0.53 | 0.80 | 0.82 | 0.72 | 0.53 |
| 192 | F111-D- | 0.40 | 0.63 | 0.91 | 0.65 | 0.40 |
| | HomoCha | | | | | |
| 193 | F111HomoCha | 0.61 | 0.64 | 0.97 | 0.74 | 0.61 |
| 194 | F111HomoPhe | 1.16 | 1.26 | 1.10 | 1.17 | 1.16 |
| 195 | F111Phg | 1.89 | 2.97 | 2.53 | 2.46 | 1.89 |
| 196 | F111Nal1 | 4.29 | 3.73 | 3.03 | 3.69 | 4.29 |
| 197 | F111Nal2 | 0.70 | 0.94 | 0.59 | 0.74 | 0.70 |
| 198 | F111StyAla | 0.49 | 1.19 | 0.96 | 0.88 | 0.49 |
| 199 | F111Phe4-Me | 0.66 | 0.65 | 0.56 | 0.62 | 0.66 |
| 200 | F111Phe-2-Me | 0.79 | 1.00 | 1.17 | 0.99 | 0.79 |
| 201 | F111Phe-4-tBu | 0.69 | 0.91 | 0.74 | 0.78 | 0.69 |
| 202 | F111Phe-4-F | 3.67 | 3.87 | 2.73 | 3.42 | 3.67 |
| 203 | F111Phe-3-F | 4.59 | 5.69 | 3.42 | 4.57 | 4.59 |
| 204 | F111Phe-2-F | 1.97 | 2.17 | 2.22 | 2.12 | 1.97 |
| 205 | F111Phe-4-Cl | 0.51 | 0.77 | 0.77 | 0.68 | 0.51 |
| 206 | F111Phe-3-Cl | 2.19 | 2.95 | 2.14 | 2.43 | 2.19 |
| 207 | F111Phe-2-Cl | 4.14 | 6.30 | 5.22 | 5.22 | 4.14 |
| 208 | F111Phe-4-Br | 0.49 | 0.81 | 1.03 | 0.78 | 0.49 |
| 209 | F111Phe-3-Br | 0.57 | 1.45 | 1.18 | 1.07 | 0.57 |
| 210 | F111Phe-2-Br | 6.88 | 10.05 | 8.16 | 8.36 | 6.88 |
| 211 | F111f | 0.46 | 0.76 | 0.77 | 0.66 | 0.46 |
| 212 | F111BIP | 0.46 | 0.92 | 0.95 | 0.78 | 0.46 |
| 213 | F111Phe-4-Me | 0.64 | 0.89 | 0.90 | 0.81 | 0.64 |
| 214 | T110E | 1.99 | 2.57 | 1.55 | 2.04 | 1.99 |
| 215 | T110D | 1.09 | 1.42 | 1.18 | 1.23 | 1.09 |
| 216 | T110CysAc. | 0.45 | 0.59 | 0.90 | 0.65 | 0.45 |
| 217 | T110Api | 2.98 | 2.87 | 2.18 | 2.68 | 2.98 |
| 218 | T110e | 0.55 | 0.78 | 0.78 | 0.71 | 0.55 |
| 219 | T110d | 0.25 | 0.63 | 0.76 | 0.55 | 0.25 |
| 220 | T110t | 0.70 | 0.79 | 0.67 | 0.72 | 0.70 |
| 221 | T110AlloThr | 3.41 | 2.51 | 2.05 | 2.66 | 3.41 |
| 222 | T109AlloThr | 0.78 | 0.58 | 0.74 | 0.70 | 0.78 |
| 223 | T109t | 0.46 | 0.81 | 0.80 | 0.69 | 0.46 |
| 224 | E108Glu(OAll) | 1.28 | 1.20 | 1.15 | 1.21 | 1.28 |
| 225 | E108Orn | 0.74 | 0.94 | 1.05 | 0.91 | 0.74 |
| 226 | E108CysAcid | 1.21 | 1.11 | 1.27 | 1.2 | 1.21 |
| 227 | E108D | 0.79 | 0.93 | 0.69 | 0.81 | 0.79 |
| 228 | E108e | 0.88 | 0.89 | 0.81 | 0.86 | 0.88 |
| 229 | E108d | 0.26 | 0.97 | 0.87 | 0.70 | 0.26 |
| 230 | E108Q | 2.15 | 1.33 | 1.70 | 1.73 | 2.15 |
| 231 | E108HomoGln | 1.45 | 1.49 | 1.39 | 1.44 | 1.45 |
| 232 | E108HomoSer | 0.85 | 1.03 | 1.19 | 1.02 | 0.85 |
| 233 | L107HomoCha | 1.47 | 1.37 | 1.93 | 1.59 | 1.47 |
| 234 | L107Cha | 3.49 | 3.17 | 1.90 | 2.86 | 3.49 |
| 235 | L107-D-Orn | 0.74 | 0.81 | 1.04 | 0.86 | 0.74 |
| 236 | L107HomoLeu | 2.66 | 2.67 | 2.02 | 2.45 | 2.66 |
| 237 | L107NLe | 3.96 | 3.25 | 3.60 | 3.60 | 3.96 |
| 238 | L107CycLeu | 0.63 | 0.91 | 0.53 | 0.69 | 0.63 |
| 239 | L107Aib | 0.44 | 0.78 | 0.76 | 0.66 | 0.44 |
| 240 | L107Abu | 4.09 | 2.83 | 2.37 | 3.1 | 4.09 |
| 241 | L107V | 2.07 | 3.19 | 1.95 | 2.4 | 2.07 |
| 242 | L107NVa | 3.47 | 2.57 | 2.15 | 2.73 | 3.47 |
| 243 | L107Tle | 2.73 | 4.15 | 3.04 | 3.31 | 2.73 |
| 244 | L107v | 0.64 | 0.94 | 0.7 | 0.76 | 0.64 |
| 245 | L107-D- | 0.46 | 1.21 | 1.08 | 0.92 | 0.46 |
| | HomoCha | | | | | |
| 246 | L107I | 0.96 | 2.10 | 1.33 | 1.46 | 0.96 |
| 247 | H106Phe-4- | 3.74 | 7.31 | 2.43 | 4.49 | 3.74 |
| | CH₂NH₂ | | | | | |
| 248 | H106Phe-3- | 4.60 | 6.25 | 5.56 | 5.47 | 4.6 |
| | CH₂NH₂ | | | | | |
| 249 | H106Phe-4- | 3.00 | 5.27 | 3.35 | 3.87 | 3 |
| | NH₂ | | | | | |
| 250 | H106Phe-4-N₃ | 2.54 | 3.85 | 3.04 | 3.14 | 2.54 |
| 251 | H106PyA-3 | 2.66 | 4.37 | 2.88 | 3.3 | 2.66 |
| 252 | H106PyA-4 | 5.02 | 6.87 | 3.61 | 5.17 | 5.02 |
| 253 | H106Phe- | 1.77 | 2.55 | 2.53 | 2.28 | 1.77 |
| | FurAla | | | | | |
| 254 | H106Cha | 0.67 | 1.27 | 0.83 | 0.92 | 0.67 |
| 255 | H106Thi | 2.58 | 2.67 | 2.77 | 2.67 | 2.58 |
| 256 | H106His(1-Me) | 1.31 | 1.76 | 1.39 | 1.49 | 1.31 |
| 257 | H106His(3-Me) | 4.41 | 2.68 | 2.61 | 3.23 | 4.41 |
| 258 | H106K | 5.49 | 5.04 | 3.2 | 4.58 | 5.49 |
| 259 | H106HomoCit | 1.88 | 1.18 | 1.56 | 1.54 | 1.88 |
| 260 | H106Orn | 6.66 | 5.34 | 3.76 | 5.25 | 6.66 |
| 261 | H106HomoLys | 2.33 | 1.75 | 2.09 | 2.06 | 2.33 |
| 262 | H106R | 9.24 | 5.86 | 6.27 | 7.12 | 9.24 |
| 263 | H106Y | 4.82 | 4.8 | 4.5 | 4.71 | 4.82 |
| 265 | H106Agp | 0.59 | 0.90 | 0.92 | 0.81 | 0.59 |
| 266 | H106HomoArg | 3.63 | 3.51 | 3.86 | 3.67 | 3.63 |
| 267 | T105Phe-4-Cl | 2.31 | 1.79 | 2.23 | 2.11 | 2.31 |
| 268 | T105Phe-3-Cl | 1.72 | 2.01 | 1.67 | 1.80 | 1.72 |
| 269 | T105Phe-2-Cl | 2.92 | 2.06 | 2.17 | 2.38 | 2.92 |
| 270 | T105-D-Phe-4- | 1.12 | 1.23 | 0.89 | 1.08 | 1.12 |
| | NH₂ | | | | | |
| 271 | T105HomoPhe | 2.13 | 2.08 | 2.36 | 2.19 | 2.13 |
| 272 | T105K | 3.8 | 2.96 | 2.23 | 2.99 | 3.80 |
| 273 | T105h | 3.32 | 3.03 | 3.18 | 3.18 | 3.32 |
| 274 | T105Phe-4- | 2.44 | 3.17 | 3.08 | 2.9 | 2.44 |
| | NH₂ | | | | | |
| 275 | T105AlloThr | 4.07 | 3.89 | 3.20 | 3.72 | 4.07 |
| 276 | T105t | 2.27 | 2.35 | 2.76 | 2.46 | 2.27 |
| 277 | T105R | 4.53 | 4.89 | 4.32 | 4.58 | 4.53 |
| 278 | T105F | 2.32 | 2.01 | 2.28 | 2.20 | 2.32 |
| 279 | T105Y | 2.59 | 3.53 | 2.19 | 2.77 | 2.59 |

TABLE 12
Fluorescence raw data (10⁶) of the non-proteogenic
AA scan for the Hit1(T112W T116E) peptide scaffold.

| SEQ | | | | | | |
|---|---|---|---|---|---|---|
| ID | | Fluor. | Fluor. | Fluor. | | |
| NO | Mutation | Pep. 1 | Pep. 2 | Pep. 3 | Average | Std.dev |
| 12 | Cyclic WT | 3.41 | 3.68 | 3.24 | 3.44 | 0.22 |
| 14 | T112W T116E | 9.88 | 12.86 | 9.36 | 10.70 | 1.89 |
| 138 | F111V | 0.28 | 0.62 | 0.91 | 0.60 | 0.31 |
| 137 | Linear WT | 0.84 | 2.16 | 1.58 | 1.52 | 0.66 |
| 280 | T116HomoSer | 4.90 | 7.34 | 4.81 | 5.68 | 1.43 |
| 281 | T116HomoGln | 5.00 | 6.38 | 4.93 | 5.44 | 0.82 |
| 282 | T116E(OAll) | 2.63 | 5.67 | 3.54 | 3.95 | 1.56 |
| 284 | T116e | 6.63 | 8.47 | 4.77 | 6.62 | 1.85 |
| 285 | T116D | 8.66 | 12.43 | 7.98 | 9.69 | 2.4 |
| 286 | T116d | 6.40 | 6.40 | 4.93 | 5.91 | 0.85 |
| 287 | T116Cys.Acid | 6.51 | 9.01 | 6.21 | 7.24 | 1.54 |
| 288 | T116Api | 6.62 | 9.06 | 6.00 | 7.23 | 1.62 |
| 289 | T116AlloThr | 5.40 | 7.41 | 5.46 | 6.09 | 1.14 |
| 290 | T112Y | 9.38 | 9.06 | 8.23 | 8.89 | 0.6 |
| 291 | T112y | 1.94 | 1.86 | 1.82 | 1.88 | 0.06 |
| 293 | T112w | 2.63 | 1.94 | 2.03 | 2.2 | 0.38 |
| 294 | T112Naphtoic | 1.41 | 1.95 | 1.39 | 1.59 | 0.32 |
| 295 | T112Nal2 | 7.56 | 6.76 | 7.27 | 7.2 | 0.41 |
| 296 | T112Nal1 | 6.35 | 14.59 | 9.89 | 10.28 | 4.13 |
| 297 | T112IndaG | 4.27 | 5.59 | 3.66 | 4.5 | 0.99 |
| 298 | T112HomoCha | 5.21 | 4.14 | 4.04 | 4.46 | 0.65 |
| 299 | T112Phe-4-NH | 10.2 | 8.55 | 9.3 | 9.35 | 0.82 |
| 300 | T112Dap | 7.15 | 5.67 | 5.16 | 5.99 | 1.03 |
| 301 | T112HomoPhe | 3.99 | 4.24 | 3.60 | 3.94 | 0.33 |
| 302 | T112H | 6.32 | 8.65 | 6.72 | 7.23 | 1.24 |
| 303 | T112F | 6.32 | 6.54 | 6.58 | 6.48 | 0.14 |
| 304 | T112f | 1.49 | 1.72 | 1.35 | 1.52 | 0.19 |
| 305 | T112-D- | 0.98 | 1.30 | 1.15 | 1.14 | 0.16 |
| | HomoCha | | | | | |
| 306 | T112-D-Dap | 2.18 | 2.40 | 1.86 | 2.15 | 0.27 |
| 307 | T112-D-Cit | 2.90 | 2.38 | 1.88 | 2.38 | 0.51 |
| 308 | T112-D-Cha | 1.34 | 1.19 | 0.99 | 1.17 | 0.18 |
| 309 | T112-D-Tyr(Et) | 1.04 | 1.49 | 1.53 | 1.35 | 0.27 |
| 310 | T112Cit | 5.67 | 8.59 | 6.31 | 6.85 | 1.54 |
| 311 | T112Cha | 7.04 | 4.63 | 4.21 | 5.29 | 1.53 |
| 312 | T112BIP | 10.38 | 7.56 | 8.38 | 8.77 | 1.45 |
| 313 | T112AlloThr | 6.12 | 4.37 | 3.48 | 4.66 | 1.34 |
| 314 | T112Aic | 3.09 | 3.43 | 2.7 | 3.07 | 0.37 |
| 315 | T112-Phe-2- | 3.2 | 8.73 | 7.92 | 6.62 | 2.99 |
| | Me | | | | | |
| 316 | T112-D-Phe- | 1.65 | 0.83 | 1.14 | 1.21 | 0.41 |
| | (3,4Cl) | | | | | |
| 317 | T112Trp-2-Me | 6.3 | 8.79 | 9.84 | 8.31 | 1.82 |
| 318 | T110t | 0.97 | 1.22 | 0.75 | 0.98 | 0.24 |
| 319 | T110E | 11.16 | 14.69 | 11.08 | 12.31 | 2.06 |
| 320 | T110e | 0.64 | 1.23 | 0.77 | 0.88 | 0.31 |
| 321 | T110D | 4.99 | 4.74 | 5.77 | 5.17 | 0.54 |
| 322 | T110d | 0.85 | 1.00 | 1.12 | 0.99 | 0.14 |
| 323 | T110CysAcid | 1.92 | 2.46 | 1.63 | 2.00 | 0.42 |
| 324 | T110Api | 16.5 | 12.64 | 16.99 | 15.38 | 2.38 |
| 325 | T110AlloThr | 7.79 | 7.23 | 9.29 | 8.10 | 1.07 |
| 326 | T109t | 0.97 | 1.05 | 0.9 | 0.97 | 0.07 |
| 327 | T109AlloThr | 0.96 | 0.76 | 0.9 | 0.87 | 0.10 |
| 328 | T105Y | 8.80 | 8.35 | 6.95 | 8.03 | 0.96 |
| 329 | T105t | 7.20 | 6.61 | 7.28 | 7.03 | 0.37 |
| 330 | T105R | 9.18 | 8.64 | 6.31 | 8.05 | 1.52 |
| 331 | T105Phe-4Cl | 5.31 | 3.62 | 3.90 | 4.28 | 0.91 |
| 332 | T105Phe-3Cl | 6.31 | 5.56 | 3.49 | 5.12 | 1.46 |
| 333 | T105Phe-2Cl | 6.91 | 6.2 | 4.36 | 5.82 | 1.32 |
| 334 | T105Phe-4- | 8.30 | 7.40 | 7.01 | 7.57 | 0.66 |
| | NH₂ | | | | | |
| 335 | T105K | 7.37 | 4.94 | 6.59 | 6.30 | 1.24 |
| 336 | T105HomoPhe | 6.47 | 5.22 | 4.07 | 5.26 | 1.2 |
| 337 | T105h | 9.59 | 5.17 | 6.83 | 7.20 | 2.24 |
| 338 | T105F | 7.52 | 6.13 | 5.90 | 6.52 | 0.88 |
| 339 | T105-D-Phe-4- | 2.55 | 1.87 | 1.63 | 2.02 | 0.48 |
| | NH₂ | | | | | |
| 340 | T105AlloThr | 11.64 | 8.32 | 10.12 | 10.03 | 1.66 |
| 341 | L107V | 6.41 | 7.83 | 6.50 | 6.91 | 0.79 |
| 342 | L107v | 1.30 | 1.27 | 0.98 | 1.18 | 0.17 |
| 343 | L107Tle | 8.82 | 8.24 | 6.8 | 7.96 | 1.04 |
| 344 | L107Nval | 11.16 | 10.46 | 9.94 | 10.52 | 0.61 |
| 345 | L107Nleu | 5.11 | 8.55 | 7.06 | 6.91 | 1.73 |
| 346 | L107I | 6.26 | 3.67 | 5.56 | 5.16 | 1.34 |
| 347 | L107HomoLeu | 8.41 | 6.45 | 7.34 | 7.40 | 0.98 |
| 348 | L107HomoCha | 3.42 | 3.89 | 3.69 | 3.67 | 0.23 |
| 349 | L107-D-Orn | 0.81 | 1.04 | 1.07 | 0.98 | 0.14 |
| 350 | L107-D- | 1.62 | 1.69 | 0.92 | 1.41 | 0.43 |
| | HomoCha | | | | | |
| 351 | L107CycLeu | 0.99 | 1.12 | 0.99 | 1.03 | 0.07 |
| 352 | L107Cha | 8.39 | 5.50 | 6.15 | 6.68 | 1.52 |
| 353 | L107Aib | 0.81 | 0.70 | 0.72 | 0.74 | 0.06 |
| 354 | L107Abu | 10.91 | 10.14 | 7.53 | 9.53 | 1.77 |
| 355 | H106Y | 11.27 | 16.44 | 15.6 | 14.44 | 2.77 |
| 356 | H106Thi | 7.99 | 10.21 | 8.77 | 8.99 | 1.13 |
| 357 | H106R | 12.17 | 20.19 | 17.95 | 16.77 | 4.14 |
| 358 | H106Orn | 10.51 | 18.07 | 18.24 | 15.61 | 4.41 |
| 359 | H106Phe-4- | 8.05 | 11.73 | 10.58 | 10.12 | 1.88 |
| | NH₂ | | | | | |
| 360 | H106K | 10.06 | 15.29 | 13.61 | 12.98 | 2.67 |
| 361 | H106K | 10.4 | 14.29 | 12.89 | 12.53 | 1.97 |
| 362 | H106HomoLys | 5.97 | 9.26 | 7.94 | 7.72 | 1.66 |
| 363 | H106HomoCit | 4.57 | 6.23 | 6.52 | 5.77 | 1.05 |
| 364 | H106HomoArg | 8.21 | 10.77 | 6.82 | 8.60 | 2.00 |
| 365 | H106His(3-Me) | 9.95 | 13.6 | 13.94 | 12.5 | 2.21 |
| 366 | H106His(1-Me) | 3.90 | 5.44 | 5.03 | 4.79 | 0.80 |
| 367 | H106Phe- | 4.65 | 6.64 | 5.67 | 5.65 | 1.00 |
| | FurAla | | | | | |
| 368 | H106PyA-4 | 15.43 | 18.52 | 13.61 | 15.85 | 2.48 |
| 369 | H106Phe-4-N₃ | 8.42 | 11.51 | 7.32 | 9.08 | 2.18 |
| 370 | G115R(Me₂) | 8.05 | 9.20 | 5.61 | 7.62 | 1.83 |
| 371 | H106Phe-4- | 12.72 | 14.78 | 12.63 | 13.38 | 1.22 |
| | CH₂N₂ | | | | | |
| 372 | G115R(Me) | 7.75 | 10.48 | 5.24 | 7.82 | 2.62 |
| 373 | G115R | 8.93 | 12.59 | 8.98 | 10.17 | 2.10 |
| 374 | G115P | 10.76 | 11.96 | 7.81 | 10.17 | 2.13 |
| 375 | G115p | 10.65 | 11.01 | 7.95 | 9.87 | 1.68 |
| 376 | G115A | 9.54 | 12.55 | 9.00 | 10.36 | 1.91 |
| 377 | G115a | 9.29 | 9.30 | 6.90 | 8.50 | 1.38 |
| 378 | G113HomoGln | 6.39 | 3.62 | 3.86 | 4.62 | 1.54 |
| 379 | G113HomoCit | 2.78 | 3.13 | 3.09 | 3.00 | 0.19 |
| 380 | G113-D-Cit | 15.26 | 9.15 | 11.05 | 11.82 | 3.13 |
| 381 | G113Cit | 6.38 | 3.62 | 4.39 | 4.80 | 1.43 |
| 382 | F111StyAla | 1.29 | 1.54 | 1.13 | 1.32 | 0.21 |
| 383 | F111Phg | 48.37 | 56.9 | 54.44 | 53.24 | 4.39 |
| 384 | F111Phe-4-F | 8.53 | 8.64 | 7.99 | 8.39 | 0.35 |
| 385 | F111Phe-4-Cl | 1.43 | 1.37 | 1.07 | 1.29 | 0.19 |
| 386 | F111Phe-4-Br | 1.02 | 1.02 | 0.61 | 0.88 | 0.24 |
| 387 | F111Phe-3-F | 18.66 | 15.33 | 10.89 | 14.96 | 3.90 |
| 388 | F111Phe-3-Cl | 7.84 | 7.03 | 6.31 | 7.06 | 0.76 |
| 389 | F111Phe-3-Br | 4.46 | 3.43 | 3.71 | 3.87 | 0.53 |
| 390 | F111Phe-2-F | 6.92 | 5.92 | 5.66 | 6.17 | 0.66 |
| 391 | F111Phe-2-Cl | 18.5 | 16.75 | 12.98 | 16.08 | 2.82 |
| 392 | F111Phe-2-Br | 15.33 | 18.55 | 15.56 | 16.48 | 1.80 |
| 393 | F111Nal2 | 1.31 | 0.98 | 1.39 | 1.23 | 0.22 |
| 394 | F111Nal1 | 7.25 | 5.31 | 5.40 | 5.99 | 1.10 |
| 395 | F111L- | 0.74 | 0.93 | 1.07 | 0.91 | 0.17 |
| | HomoCha | | | | | |
| 396 | F111HomoPhe | 1.81 | 1.58 | 1.54 | 1.64 | 0.14 |
| 397 | F111Phe(4- | 1.18 | 1.22 | 1.07 | 1.16 | 0.08 |
| | tBu) | | | | | |
| 398 | F111f | 1.05 | 1.04 | 0.93 | 1.01 | 0.07 |
| 399 | F111-D- | 1.08 | 0.97 | 0.98 | 1.01 | 0.06 |
| | Homocha | | | | | |
| 400 | F111-D-Cha | 1.06 | 0.83 | 1.01 | 0.97 | 0.12 |
| 401 | F111Cha | 1.27 | 1.46 | 1.28 | 1.34 | 0.10 |
| 402 | F111BIP | 0.74 | 1.94 | 1.40 | 1.36 | 0.60 |
| 403 | F111Phe-4-Me | 1.09 | 0.83 | 1.08 | 1.00 | 0.15 |
| 404 | F111Phe-3-Me | 0.85 | 0.84 | 1.26 | 0.98 | 0.24 |
| 405 | F111Phe-2-Me | 1.26 | 1.43 | 0.85 | 1.18 | 0.30 |
| 406 | E108R | 4.50 | 3.88 | 3.42 | 3.94 | 0.54 |
| 407 | E108Q | 3.78 | 3.73 | 2.41 | 3.31 | 0.78 |
| 408 | E108Orn | 1.42 | 1.79 | 1.77 | 1.66 | 0.21 |
| 409 | E108HomoSer | 4.68 | 4.67 | 2.76 | 4.04 | 1.11 |
| 410 | E108HomoGln | 4.18 | 4.37 | 2.99 | 3.85 | 0.74 |
| 411 | E108Glu(Oall) | 2.17 | 2.27 | 1.95 | 2.13 | 0.16 |
| 412 | E108e | 0.77 | 1.28 | 0.80 | 0.95 | 0.29 |
| 413 | E108D | 1.73 | 2.06 | 2.02 | 1.94 | 0.18 |
| 414 | E108d | 1.07 | 0.85 | 1.10 | 1.01 | 0.14 |
| 415 | E108CysAcid | 3.03 | 3.91 | 2.99 | 3.31 | 0.52 |
| 416 | D114E | 15.43 | 8.69 | 7.82 | 10.65 | 4.17 |
| 417 | D114e | 9.55 | 7.32 | 6.64 | 7.83 | 1.52 |
| 418 | D114d | 5.94 | 6.23 | 4.00 | 5.39 | 1.21 |

### Example 15

### In Vitro Plasmin Stability of Ligands

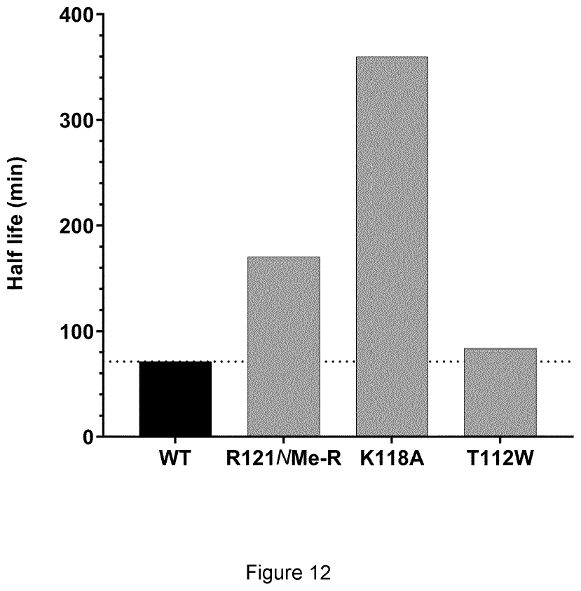

The in vitro plasmin stability of peptides with SEQ ID NOs: 12, 68, 99 and 163 were determined by incubating 100 μM of ligand in phosphate buffered saline (PBS) supplemented with plasmin (10 μg/mL) at 37° C. for 0 to 360 minutes. At selected time points during the incubation, the ligands were extracted from 80 μL of assay matrix by treatment with 80 μL of 50% acetonitrile (ACN). The samples were filtered and analysed by UPLC to determine the amount of ligand remaining. LC-MS analysis was performed to confirm ligand integrity and identify cleavage sites. The results are presented in table 13 and FIG. 12.

TABLE 13
In vitro plasmin stability, half-life times pre-
sented as mean, cyclic nNOS β-hairpin peptides
(FIG. 12).

| SEQ | | |
|---|---|---|
| ID | | T1/2 |
| NO | Sequence | [min] |
| 12 | cyclo-(THLETTFTGDGTPKTIRVTQpG(Qα)) | 71 |
| 68 | cyclo-(THLETTFTGDGTPATIRVTQpG(Qα)) | 360 |
| 99 | cyclo-(THLETTFTGDGTPKTI [symbol] -Me-RVTQpG(Qα)) | 170 |
| 163 | cyclo-(THLETTFWGDGTPKTIRVTQpG(Qα)) | 84 |

This example describes how to determine the in vitro plasmin stability of cyclic nNOS β-hairpin peptides. In conclusion, degradation prone residues R121 and K118 can be substituted to significantly improve plasmin stability. Furthermore, substitutions that significantly improve the compounds affinity to PSD-95 (T112VV) do not compromise stability.

### Example 16

### Determination of Membrane Permeability and Cellular Uptake of Ligands by CAPA

The membrane permeability of cyclo-(THLETTFTGDGTP(K-CA)TIRVTQpG(Qα)) (SEQ ID NO: 427) was determined in HeLa cells stably expressing HaloGFP exclusively located in the cytosol. Cells were seeded at a density of 40.000 cells/well one day prior to the experiment. After the growth media was aspirated and replaced by 100 μL of Opti-MEM, 25 μL of a prepared serial dilution of the ligand in Opti-MEM was added to the cells (constant DMSO concentration), and the plate was incubated for 4 h at 37 ° C. and 5% CO2. The contents of the wells were aspirated, and cells were washed with fresh Opti-MEM for 15 min. After aspiration of the wash, the cells were incubated with TAMRA-CA (5 μM) for 15 min. After aspiration of the chase solution, cells were washed with Opti MEM for 30 min. Following removal of the wash, cells were trypsinized, resuspended in PBS (2% FBS), and analyzed using a benchtop flow cytometer. Using no ligand and no TAMRA-CA control well, the obtained fluorescence intensity data was normalized and plotted as dose-response curves.

This example describes how to determine the membrane permeability and cellular uptake of CA-tagged cyclic nNOS β-hairpin peptides. The membrane permeability and cellular uptake value (CP₅₀) values represent half-maximum red fluorescence which behaves inverse to cell penetration of the ligand. The CP₅₀ value for cyclo-(THLETTFTGDGTP(K-CA)TIRVTQpG(Qα)) (SEQ ID NO: 427) was 31.1±2.1 μM. In conclusion, the presented cyclic peptide exhibited suitable cellular uptake for medical applications.

Sequences

| SEQ ID | | |
|---|---|---|
| NO | Peptide sequence | Comment |
| 1 | TX₁LETX₂X₃X₄GX₅X₆X₇PX₈TIRVX₉Q | X₁ is H, H-3Me or |
| | | PyA-4; |
| | | X₂ is T, S, D or E; |
| | | X₃ is F, F-2-Br, F-2-Cl |
| | | or F-3-F; |
| | | X₄ is W, Nal, or |
| | | absent; |
| | | X₅ is D or N-Me-D; |
| | | X₆ is G, A or P; |
| | | X₇ is E or D; |
| | | X₈ is K or N-Me-K; |
| | | X₉ is T or N-Me-T; |
| 2 | TX₁LETX₂X₃X₄GX₅X₆X₇PX₈TIRVX₉QpGX₁₀ | X₁ is H, H-3Me or |
| | | PyA-4; |
| | | X₂ is T, S, D or E; |
| | | X₃ is F, F-2-Br, F-2-Cl |
| | | or F-3-F; |
| | | X₄ is W, Nal, or |
| | | absent; |
| | | X₅ is D or N-Me-D; |
| | | X₆ is G, A or P; |
| | | X₇ is E or D; |
| | | X₈ is K or N-Me-K; |
| | | X₉ is T or N-Me-T; |
| | | X₁₀ is C, Q or E; |
| 3 | TX₁LETX₂X₃GX₅X₆X₇PX₈TIRVX₉Q | X₁ is H, H-3Me or |
| | | PyA-4; |
| | | X₂ is T, S, D or E; |
| | | X₃ is F, F-2-Br, F-2-Cl |
| | | or F-3-F; |
| | | X₅ is D or N-Me-D; |
| | | X₆ is G, A or P; |
| | | X₇ is E or D; |
| | | X₈ is K or N-Me-K; |
| | | X₉ is T or N-Me-T; |
| 4 | TX₁LETX₂X₃GX₅X₆X₇PX₈TIRVX₉QpGX₁₀ | X₁ is H, H-3Me or |
| | | PyA-4; |
| | | X₂ is T, S, D or E; |
| | | X₃ is F, F-2-Br, F-2-Cl |
| | | or F-3-F; |
| | | X₅ is D or N-Me-D; |
| | | X₆ is G, A or P; |
| | | X₇ is E or D; |
| | | X₈ is K or N-Me-K; |
| | | X₉ is T or N-Me-T; |
| | | X₁₀ is C, Q or E; |
| 5 | TX₁LETTFX₄GX₅X₆X₇PX₈TIRVX₉QpGX₁₀ | X₁ is H, H-3Me or |
| | | PyA-4; |
| | | X₄ is W, Nal, or |
| | | absent; |
| | | X₅ is D or N-Me-D; |
| | | X₆ is G, A or P; |
| | | X₇ is E or D; |
| | | X₈ is K or NV-Me-K; |
| | | X₉ is T or N-Me-T; |
| | | X₁₀ is C, Q or E; |
| 6 | TX₁LETTFGX₅X₆X₇PX₈TIRVX₉QpGX₁₀ | X₁ is H, H-3Me or |
| | | PyA-4; |
| | | X₅ is D or N-Me-D; |
| | | X₆ is G, A or P; |
| | | X₇ is E or D; |
| | | X₈ is K or N-Me-K; |
| | | X₉ is T or N-Me-T; |
| | | X₁₀ is C, Q or E; |
| 7 | TX₁LETTFX₄GDGX₇PX₈TIRVX₉Q | X₁ is H, or PyA-4; |
| | | X₄ is W or Nai; |
| | | X₇ is E or D; |
| | | X₈ is K or N-Me-K; |
| | | and |
| | | X₉ is T or N-Me-T; |
| 8 | THLETTFWGDGE | |
| 9 | THLETTFWGDGD | |
| 10 | THLETTF(Nal)GDGE | |
| 11 | T(PyA-4)LETTFWGDGE | |
| 12 | Cyclo-(THLETTFTGDGTPKTIRVTQpG(Qα)) | Wild type mimic |
| 13 | TX₁LETTFX₄GDGEPKTIRVTQpGX₁₀ | X₁ is Hor H-3Me; |
| | | X₄ is W or absent; |
| | | X₁₀ is C, Q or E; |
| 14 | Cyclo-(THLETTFWGDGEPKTIRVTQPG(Qα)) | Hit1 |
| 15 | Cyclo-(THLETTFGDGEPKTIRVTQpG(Qα)) | |
| 16 | Cyclo-(TH(3-Me)LETTFWGDGEPKTIRVTQpG(Qα)) | |
| 17 | Cyclo-(TH(3-Me)LETTFGDGEPKTIRVTQpG(Qα)) | |
| 18 | Cyclo-(THLETTFWGDGTPKTIRVTQpG(Qα)) | |
| 19 | Cyclo-(THLETTFDGDGTPKTIRVTQpG(Qα)) | |
| 20 | Cyclo-(THLETTFHGDGTPKTIRVTQpG(Qα)) | |
| 21 | Cyclo-(THLETTVTGDGTPKTIRVTQpG(Qα)) | |
| 22 | Cyclo-(THKETTFTGDGTPKTIRVTQpG(Qα)) | |
| 23 | Cyclo-(TELETTFTGDGTPKTIRVTQpG(Qα)) | |
| 24 | Cyclo-(YHLETTFTGDGTPKTIRVTQpG(Qα)) | |
| 25 | Cyclo-(GHLETTFTGDGTPKTIRVTQpG(Qα)) | |
| 26 | Cyclo-(TYLETTFTGDGTPKTIRVTQpG(Qα)) | |
| 27 | Cyclo-(TFLETTFTGDGTPKTIRVTQpG(Qα)) | |
| 28 | Cyclo-(THLETTFTGKGTPKTIRVTQpG(Qα)) | |
| 29 | Cyclo-(THLETTFTGPGTPKTIRVTQpG(Qα)) | |
| 30 | Cyclo-(THLETTFTGDPTPKTIRVTQpG(Qα)) | |
| 31 | Cyclo-(THLETTFTGDGPPKTIRVTQpG(Qα)) | |
| 32 | Cyclo-(THLETTFTGDGDPKTIRVTQpG(Qα)) | |
| 33 | Cyclo-(THLETTFTGDGEPKTIRVTQpG(Qα)) | |
| 34 | Cyclo-(THLETTFTGRGTPKTIRVTQpG(Qα)) | |
| 35 | Cyclo-(THLETTFTGDRTPKTIRVTQpG(Qα)) | |
| 36 | Cyclo-(THLETTFTGDGWPKTIRVTQpG(Qα)) | |
| 37 | Cyclo-(THLETTFMGDGTPKTIRVTQpG(Qα)) | |
| 38 | Cyclo-(THLETTFTGDGCPKTIRVTQpG(Qα)) | |
| 39 | Cyclo-(THLETTFTGDGTPKNIRVTQpG(Qα)) | |
| 40 | Cyclo-(THLETTFTGDGNPKTIRVTQpG(Qα)) | |
| 41 | Cyclo-(THLETTFTGDGTPKTRRVTQpG(Qα)) | |
| 42 | Cyclo-(THLETTFTGDGTPKTYRVTQpG(Qα)) | |
| 43 | Cyclo-(THLETTFTGDGTPKTIRRTQpG(Qα)) | |
| 44 | Cyclo-(THLETTFTGDGTPKTIRVRQpG(Qα)) | |
| 45 | Cyclo-(THLETTFTGDGTPKTIRVRRpG(Qα)) | |
| 46 | Cyclo-(THLETTFTGDGTPKTIRFTQpG(Qα)) | |
| 47 | Cyclo-(THLETTFTGDGTPKTIRYTQpG(Qα)) | |
| 48 | Cyclo-(THLETTFTGDGTPKTIRVFQpG(Qα)) | |
| 49 | Cyclo-(THLETTFTGDGTPKTIRVYQpG(Qα)) | |
| 50 | Cyclo-(THLETTFTGDGTPKSIRVTQpG(Qα)) | |
| 51 | Cyclo-(THLETTFTGGGTPKTIRVTQpG(Qα)) | |
| 52 | Cyclo-(THLESTFTGDGTPKTIRVTQpG(Qα)) | |
| 53 | Cyclo-(THLETEFTGDGTPKTIRVTQpG(Qα)) | |
| 54 | Cyclo-(THLETDFTGDGTPKTIRVTQpG(Qα)) | |
| 55 | Cyclo-(AHLETTFTGDGTPKTIRVTQpG(Qα)) | |
| 56 | Cyclo-(TALETTFTGDGTPKTIRVTQpG(Qα)) | |
| 57 | Cyclo-(THAETTFTGDGTPKTIRVTQpG(Qα)) | |
| 58 | Cyclo-(THLATTFTGDGTPKTIRVTQpG(Qα)) | |
| 59 | Cyclo-(THLEATFTGDGTPKTIRVTQpG(Qα)) | |
| 60 | Cyclo-(THLETAFTGDGTPKTIRVTQpG(Qα)) | |
| 61 | Cyclo-(THLETTATGDGTPKTIRVTQpG(Qα)) | |
| 62 | Cyclo-(THLETTFAGDGTPKTIRVTQpG(Qα)) | |
| 63 | Cyclo-(THLETTFTADGTPKTIRVTQpG(Qα)) | |
| 64 | Cyclo-(THLETTFTGAGTPKTIRVTQpG(Qα)) | |
| 65 | Cyclo-(THLETTFTGDATPKTIRVTQpG(Qα)) | |
| 66 | Cyclo-(THLETTFTGDGAPKTIRVTQpG(Qα)) | |
| 67 | Cyclo-(THLETTFTGDGTAKTIRVTQpG(Qα)) | |
| 68 | Cyclo-(THLETTFTGDGTPATIRVTQpG(Qα)) | |
| 69 | Cyclo-(THLETTFTGDGTPKAIRVTQpG(Qα)) | |
| 70 | Cyclo-(THLETTFTGDGTPKTARVTQpG(Qα)) | |
| 71 | Cyclo-(THLETTFTGDGTPKTIAVTQpG(Qα)) | |
| 72 | Cyclo-(THLETTFTGDGTPKTIRATQpG(Qα)) | |
| 73 | Cyclo-(THLETTFTGDGTPKTIRVAQpG(Qα)) | |
| 74 | Cyclo-(THLETTFTGDGTPKTIRVTApG(Qα)) | |
| 75 | Cyclo-(THLETTFGDGTPKTIRVTQpG(Qα)) | ΔT112 |
| 76 | Cyclo-(THLETTFTPKTIRVTQpG(Qα)) | ΔT112, ΔG113, |
| | | ΔD114, ΔG115 |
| 77 | Cyclo-(THLETTFTGDGPKTIRVTQpG(Qα)) | ΔT116 |
| 78 | Cyclo-(THLETTFTGGTPKTIRVTQpG(Qα)) | ΔD114 |
| 79 | Cyclo-(THLETTFTGDTPKTIRVTQpG(Qα)) | ΔG113 |
| 80 | Cyclo-(THLEAlloTTFTGDGTPKTIRVTQpG(Qα)) | |
| 81 | Cyclo-(THLETAlloTFTGDGTPKTIRVTQpG(Qα)) | |
| 82 | Cyclo-(AlloTHLETTFTGDGTPKTIRVTQpG(Qα)) | |
| 83 | Cyclo-(THLETTFTGDGTPKAlloTIRVTQpG(Qα)) | |
| 84 | Cyclo-( [symbol] -Me-THLETTFTGDGTPKTIRVTQpG(Qα)) | |
| 85 | Cyclo-( [symbol] -Me-HLETTFTGDGTPKTIRVTQpG(Qα)) | |
| 86 | Cyclo-(T [symbol] -Me-LETTFTGDGTPKTIRVTQpG(Qα)) | |
| 87 | Cyclo-(TH [symbol] -Me-ETTFTGDGTPKTIRVTQpG(Qα)) | |
| 88 | Cyclo-(THL [symbol] -Me-TTFTGDGTPKTIRVTQpG(Qα)) | |
| 89 | Cyclo-(THLET [symbol] -Me-TFTGDGTPKTIRVTQPG(Qα)) | |
| 90 | Cyclo-(THLETT [symbol] -Me-FTGDGTPKTIRVTQpG(Qα)) | |
| 91 | Cyclo-(THLETTF [symbol] -Me-TGDGTPKTIRVTQPG(Qα)) | |
| 92 | Cyclo-(THLETTFT [symbol] -Me-GDGTPKTIRVTQpG(Qα)) | |
| 93 | Cyclo-(THLETTFTG [symbol] -Me-DGTPKTIRVTQpG(Qα)) | |
| 94 | Cyclo-(THLETTFTGD [symbol] -Me-GTPKTIRVTQpG(Qα)) | |
| 95 | Cyclo-(THLETTFTGDG [symbol] -Me-TPKTIRVTQpG(Qα)) | |
| 96 | Cyclo-(THLETTFTGDGTP [symbol] -Me-KTIRVTQpG(Qα)) | |
| 97 | Cyclo-(THLETTFTGDGTPK [symbol] -Me-TIRVTQpG(Qα)) | |
| 98 | Cyclo-(THLETTFTGDGTPKT [symbol] -Me-IRVTQpG(Qα)) | |
| 99 | Cyclo-(THLETTFTGDGTPKTI [symbol] -Me-RVTQpG(Qα)) | |
| 100 | Cyclo-(THLETTFTGDGTPKTIR [symbol] -Me-VTQpG(Qα)) | |
| 101 | Cyclo-(THLETTFTGDGTPKTIRV [symbol] -Me-TQpG(Qα)) | |
| 102 | Cyclo-(THLETTFTGDGTPKTIRVT [symbol] -Me-QpG(Qα)) | |
| 103 | Cyclo-( [symbol] THLETTFTGDGTPKTIRVTQpG(Qα)) | |
| 104 | Cyclo-(THLE [symbol] TFTGDGTPKTIRVTQpG(Qα)) | |
| 105 | Cyclo-(THLET [symbol] TFTGDGTPKTIRVTQpG(Qα)) | |
| 106 | Cyclo-(THLETTF [symbol] TGDGTPKTIRVTQpG(Qα)) | |
| 107 | Cyclo-(THLETTFTGDG [symbol] TPKTIRVTQpG(Qα)) | |
| 108 | Cyclo-(THLETTFTGDGTPK [symbol] TIRVTQpG(Qα)) | |
| 109 | Cyclo-(THLETTFTGDGTPKTIRV [symbol] TQpG(Qα)) | |
| 110 | Cyclo-(TH(3-Me)LETTFTGDGTPKTIRVTQpG(Qα)) | |
| 111 | Cyclo-(TH(1-Me)LETTFTGDGTPKTIRVTQpG(Qα)) | |
| 112 | Cyclo-(tHLETTFTGDGTPKTIRVTQpG(Qα)) | |
| 113 | Cyclo-(ThLETTFTGDGTPKTIRVTQpG(Qα)) | |
| 114 | Cyclo-(THIETTFTGDGTPKTIRVQpG(Qα)) | |
| 115 | Cyclo-(THLeTTFTGDGTPKTIRVTQpG(Qα)) | |
| 116 | Cyclo-(THLEtTFTGDGTPKTIRVTQpG(Qα)) | |
| 117 | Cyclo-(THLETtFTGDGTPKTIRVTQpG(Qα)) | |
| 118 | Cyclo-(THLETTfTGDGTPKTIRVTQpG(Qα)) | |
| 119 | Cyclo-(THLETTFtGDGTPKTIRVTQpG(Qα)) | |
| 120 | Cyclo-(THLETTFTGdGTPKTIRVTQpG(Qα)) | |
| 121 | Cyclo-(THLETTFTGDGtPKTIRVTQpG(Qα)) | |
| 122 | Cyclo-(THLETTFTGDGTpKTIRVTQpG(Qα)) | |
| 123 | Cyclo-(THLETTFTGDGTPkTIRVTQpG(Qα)) | |
| 124 | Cyclo-(THLETTFTGDGTPKtIRVTQpG(Qα)) | |
| 125 | Cyclo-(THLETTFTGDGTPKTiRVTQpG(Qα)) | |
| 126 | Cyclo-(THLETTFTGDGTPKTIrVTQpG(Qα)) | |
| 127 | Cyclo-(THLETTFTGDGTPKTIRvTQpG(Qα)) | |
| 128 | Cyclo-(THLETTFTGDGTPKTIRVtQpG(Qα)) | |
| 129 | Cyclo-(THLETTFTGDGTPKTIRVTqpG(Qα)) | |
| 130 | Cyclo-(THLETTFWGDGDPKTIRVTQpG(QG)) | |
| 131 | Cyclo-(THLETTFWGDPTPKTIRVTQpG(QG)) | |
| 132 | Cyclo-(THLETTFTGDPEPKTIRVTQpG(Qα)) | |
| 133 | Cyclo-(THLETTFWGDPEPKTIRVTQpG(Qα)) | |
| 134 | Cyclo-(THLETTFWGDAEPKTIRVTQpG(Qα)) | |
| 135 | Cyclo-(THLETTFWGdGEPKTIRVTQPG(Qα)) | |
| 136 | Cyclo-(THLETTFGdGEPKTIRVTQpG(Qα)) | ΔT112 D114d T116E |
| 137 | THLETTFTGDGTPKTIRVTQpGQE | Un-cyclized wild type |
| | | mimic |
| 138 | cyclo-(THLETTVTGDGTPKTIRVTQpG(Qα)) | Negative control |
| 139 | cyclo-(THLETTFTGDGEPKTIRVTQpG(Qα)) | |
| 140 | cyclo-(THLETTFTGDGePKTIRVTQpG(Qα)) | |
| 141 | cyclo-(THLETTFTGDGDPKTIRVTQpG(Qα)) | |
| 142 | cyclo-(THLETTFTGDGdPKTIRVTQpG(Qα)) | |
| 143 | cyclo-(THLETTFTGDGHomoSerPKTIRVTQpG(Qα)) | |
| 144 | cyclo-(THLETTFTGDGAlloThrPKTIRVTQpG(Qα)) | |
| 145 | cyclo-(THLETTFTGDGGlu(OAll)PKTIRVTQpG(Qα)) | |
| 146 | cyclo-(THLETTFTGDGCys.AcidPKTIRVTQpG(Qα)) | |
| 147 | cyclo-(THLETTFTGDGApiPKTIRVTQpG(Qα)) | |
| 148 | cyclo-(THLETTFTGDGHomoGlnPKTIRVTQpG(Qα)) | |
| 149 | cyclo-(THLETTFTGDR(Me)TPKTIRVTQpG(Qα)) | |
| 150 | cyclo-(THLETTFTGDR(Me2)TPKTIRVTQpG(Qα)) | |
| 151 | cyclo-(THLETTFTGDRTPKTIRVTQpG(Qα)) | |
| 152 | cyclo-(THLETTFTGDATPKTIRVTQpG(Qα)) | |
| 153 | cyclo-(THLETTFTGDPTPKTIRVTQpG(Qα)) | |
| 154 | cyclo-(THLETTFTGDaTPKTIRVTQpG(Qα)) | |
| 155 | cyclo-(THLETTFTGDpTPKTIRVTQpG(Qα)) | |
| 156 | cyclo-(THLETTFTGdGTPKTIRVTQpG(Qα)) | |
| 157 | cyclo-(THLETTFTGEGTPKTIRVTQpG(Qα)) | |
| 158 | cyclo-(THLETTFTGeGTPKTIRVTQpG(Qα)) | |
| 159 | cyclo-(THLETTFTD-CitDGTPKTIRVTQpG(Qα)) | |
| 160 | cyclo-(THLETTFTCitDGTPKTIRVTQpG(Qα)) | |
| 161 | cyclo-(THLETTFTHomoCitDGTPKTIRVTQpG(Qα)) | |
| 162 | cyclo-(THLETTFTHomoCitDGTPKTIRVTQpG(Qα)) | |
| 163 | cyclo-(THLETTFWGDGTPKTIRVTQpG(Qα)) | |
| 164 | cyclo-(THLETTFwGDGTPKTIRVTQpG(Qα)) | |
| 165 | cyclo-(THLETTFAlloThrGDGTPKTIRVTQpG(Qα)) | |
| 166 | cyclo-(THLETTFPhe-4-MeGDGTPKTIRVTQpG(Qα)) | |
| 167 | cyclo-(THLETTFD-Phe- | |
| | (3,4Cl)GDGTPKTIRVTQpG(Qα)) | |
| 168 | cyclo-(THLETTFPhe-4-NH2GDGTPKTIRVTQpG(Qα)) | |
| 169 | cyclo-(THLETTFBIPGDGTPKTIRVTQpG(Qα)) | |
| 170 | cyclo-(THLETTFD- | |
| | HomoChaGDGTPKTIRVTQpG(Qα)) | |
| 171 | cyclo-(THLETTFHomoChaGDGTPKTIRVTQpG(Qα)) | |
| 172 | cyclo-(THLETTFChaGDGTPKTIRVTQpG(Qα)) | |
| 173 | cyclo-(THLETTFD-ChaGDGTPKTIRVTQpG(Qα)) | |
| 174 | cyclo-(THLETTFYGDGTPKTIRVTQpG(Qα)) | |
| 175 | cyclo-(THLETTFyGDGTPKTIRVTQpG(Qα)) | |
| 176 | cyclo-(THLETTFD-Tyr-2- | |
| | (Et)ChaGDGTPKTIRVTQpG(Qα)) | |
| 177 | cyclo-(THLETTFD-CitGDGTPKTIRVTQpG(Qα)) | |
| 178 | cyclo-(THLETTFCitGDGTPKTIRVTQpG(Qα)) | |
| 179 | cyclo-(THLETTFD-DapGDGTPKTIRVTQpG(Qα)) | |
| 180 | cyclo-(THLETTFDapGDGTPKTIRVTQpG(Qα)) | |
| 181 | Cyclo-(THLETTFNal2GDGTPKTIRVTQpG(Qα)) | |
| 182 | Cyclo-(THLETTFIndaGGDGTPKTIRVTQpG(Qα)) | |
| 183 | Cyclo-(THLETTFTrp-2-MeGDGTPKTIRVTQpG(Qα)) | |
| 184 | Cyclo-(THLETTFAicGDGTPKTIRVTQpG(Qα)) | |
| 185 | Cyclo-(THLETTFFGDGTPKTIRVTQpG(Qα)) | |
| 186 | Cyclo-(THLETTFfGDGTPKTIRVTQpG(Qα)) | |
| 187 | Cyclo-(THLETTFHGDGTPKTIRVTQpG(Qα)) | |
| 188 | Cyclo-(THLETTFHomoPheGDGTPKTIRVTQpG(Qα)) | |
| 189 | Cyclo-(THLETTFNapthoic | |
| | AcidGDGTPKTIRVTQpG(Qα)) | |
| 190 | Cyclo-(THLETTChaTGDGTPKTIRVTQpG(Qα)) | |
| 191 | Cyclo-(THLETTD-ChaTGDGTPKTIRVTQpG(Qα)) | |
| 192 | Cyclo-(THLETTD- | |
| | HomoChaGDGTPKTIRVTQpG(Qα)) | |
| 193 | Cyclo-(THLETTHomoChaTGDGTPKTIRVTQpG(Qα)) | |
| 194 | Cyclo-(THLETTD- | |
| | HomoPheTGDGTPKTIRVTQpG(Qα)) | |
| 195 | Cyclo-(THLETTPhgTGDGTPKTIRVTQpG(Qα)) | |
| 196 | Cyclo-(THLETTNal1TGDGTPKTIRVTQpG(Qα)) | |
| 197 | Cyclo-(THLETTNal2TGDGTPKTIRVTQpG(Qα)) | |
| 198 | Cyclo-(THLETTStyAlaTGDGTPKTIRVTQpG(Qα)) | |
| 199 | Cyclo-(THLETTPhe-4-MeTGDGTPKTIRVTQpG(Qα)) | |
| 200 | Cyclo-(THLETTPhe-2-MeTGDGTPKTIRVTQpG(Qα)) | |
| 201 | Cyclo-(THLETTPhe-4-tBuTGDGTPKTIRVTQpG(Qα)) | |
| 202 | Cyclo-(THLETTPhe-4-FTGDGTPKTIRVTQpG(Qα)) | |
| 203 | Cyclo-(THLETTPhe-3-FTGDGTPKTIRVTQpG(Qα)) | |
| 204 | Cyclo-(THLETTPhe-2-FTGDGTPKTIRVTQpG(Qα)) | |
| 205 | Cyclo-(THLETTPhe-4-ClTGDGTPKTIRVTQpG(Qα)) | |
| 206 | Cyclo-(THLETTPhe-3-ClTGDGTPKTIRVTQpG(Qα)) | |
| 207 | Cyclo-(THLETTPhe-2-ClTGDGTPKTIRVTQpG(Qα)) | |
| 208 | Cyclo-(THLETTPhe-4-BrTGDGTPKTIRVTQpG(Qα)) | |
| 209 | Cyclo-(THLETTPhe-3-BrTGDGTPKTIRVTQpG(Qα)) | |
| 210 | Cyclo-(THLETTPhe-2-BrTGDGTPKTIRVTQpG(Qα)) | |
| 211 | Cyclo-(THLETTfTGDGTPKTIRVTQpG(Qα)) | |
| 212 | Cyclo-(THLETTBIPTGDGTPKTIRVTQpG(Qα)) | |
| 213 | Cyclo-(THLETTPhe-4-MeTGDGTPKTIRVTQpG(Qα)) | |
| 214 | Cyclo-(THLETEFTGDGTPKTIRVTQpG(Qα)) | |
| 215 | Cyclo-(THLETDFTGDGTPKTIRVTQpG(Qα)) | |
| 216 | Cyclo-(THLETCys.AcidFTGDGTPKTIRVTQpG(Qα)) | |
| 217 | Cyclo-(THLETApiFTGDGTPKTIRVTQpG(Qα)) | |
| 218 | Cyclo-(THLETeFTGDGTPKTIRVTQpG(Qα)) | |
| 219 | Cyclo-(THLETdFTGDGTPKTIRVTQpG(Qα)) | |
| 220 | Cyclo-(THLETtFTGDGTPKTIRVTQpG(Qα)) | |
| 221 | Cyclo-(THLETAlloThrFTGDGTPKTIRVTQpG(Qα)) | |
| 222 | Cyclo-(THLEAlloThrTFTGDGTPKTIRVTQpG(Qα)) | |
| 223 | Cyclo-(THLEtTFTGDGTPKTIRVTQpG(Qα)) | |
| 224 | Cyclo-(THLGlu(OAll)TTFTGDGTPKTIRVTQpG(Qα)) | |
| 225 | Cyclo-(THLOrnTTFTGDGTPKTIRVTQpG(Qα)) | |
| 226 | Cyclo-(THLCys_AcidTTFTGDGTPKTIRVTQpG(Qα)) | |
| 227 | Cyclo-(THLDTTFTGDGTPKTIRVTQpG(Qα)) | |
| 228 | Cyclo-(THLeTTFTGDGTPKTIRVTQpG(Qα)) | |
| 229 | Cyclo-(THLdTTFTGDGTPKTIRVTQpG(Qα)) | |
| 230 | Cyclo-(THLQTTFTGDGTPKTIRVTQpG(Qα)) | |
| 231 | Cyclo-(THLHomoGlnTTFTGDGTPKTIRVTQpG(Qα)) | |
| 232 | Cyclo-(THLHomoSerTTFTGDGTPKTIRVTQpG(Qα)) | |
| 233 | Cyclo-(THHomoChaETTFTGDGTPKTIRVTQpG(Qα)) | |
| 234 | Cyclo-(THChaETTFTGDGTPKTIRVTQpG(Qα)) | |
| 235 | Cyclo-(THD-OrnETTFTGDGTPKTIRVTQpG(Qα)) | |
| 236 | Cyclo-(THHomoLeuETTFTGDGTPKTIRVTQpG(Qα)) | |
| 237 | Cyclo-(THNLeuETTFTGDGTPKTIRVTQpG(Qα)) | |
| 238 | Cyclo-(THCycLeuETTFTGDGTPKTIRVTQpG(Qα)) | |
| 239 | Cyclo-(THAibETTFTGDGTPKTIRVTQpG(Qα)) | |
| 240 | Cyclo-(THAbuETTFTGDGTPKTIRVTQpG(Qα)) | |
| 241 | Cyclo-(THVETTFTGDGTPKTIRVTQpG(Qα)) | |
| 242 | Cyclo-(THNVaETTFTGDGTPKTIRVTQpG(Qα)) | |
| 243 | Cyclo-(THTleETTFTGDGTPKTIRVTQpG(Qα)) | |
| 244 | Cyclo-(THvETTFTGDGTPKTIRVTQpG(Qα)) | |
| 245 | Cyclo-(THD- | |
| | HomoChaETTFTGDGTPKTIRVTQpG(Qα)) | |
| 246 | Cyclo-(THIETTFTGDGTPKTIRVTQpG(Qα)) | |
| 247 | Cyclo-(TPhe-4- | |
| | CH2NH2LETTFTGDGTPKTIRVTQpG(Qα)) | |
| 248 | Cyclo-(TPhe-3- | |
| | CH2NH2LETTFTGDGTPKTIRVTQpG(Qα)) | |
| 249 | Cyclo-(TPhe-4-NH2LETTFTGDGTPKTIRVTQpG(Qα)) | |
| 250 | Cyclo-(TPhe-4-N3LETTFTGDGTPKTIRVTQpG(Qα)) | |
| 251 | Cyclo-(TPyA-3LETTFTGDGTPKTIRVTQpG(Qα)) | |
| 252 | Cyclo-(TPyA-4LETTFTGDGTPKTIRVTQpG(Qα)) | |
| 253 | Cyclo-(TFurAlaLETTFTGDGTPKTIRVTQpG(Qα)) | |
| 254 | Cyclo-(TChaLETTFTGDGTPKTIRVTQpG(Qα)) | |
| 255 | Cyclo-(TThiLETTFTGDGTPKTIRVTQpG(Qα)) | |
| 256 | Cyclo-(THis(1-Me)LETTFTGDGTPKTIRVTQpG(Qα)) | |
| 257 | Cyclo-(THis(3-Me)LETTFTGDGTPKTIRVTQpG(Qα)) | |
| 258 | Cyclo-(TKLETTFTGDGTPKTIRVTQpG(Qα)) | |
| 259 | Cyclo-(THomoCitLETTFTGDGTPKTIRVTQpG(Qα)) | |
| 260 | Cyclo-(TOrnLETTFTGDGTPKTIRVTQpG(Qα)) | |
| 261 | Cyclo-(THomoLysLETTFTGDGTPKTIRVTQpG(Qα)) | |
| 262 | Cyclo-(TRLETTFTGDGTPKTIRVTQpG(Qα)) | |
| 263 | Cyclo-(TYLETTFTGDGTPKTIRVTQpG(Qα)) | |
| 265 | Cyclo-(TAgpLETTFTGDGTPKTIRVTQpG(Qα)) | |
| 266 | Cyclo-(THomoArgLETTFTGDGTPKTIRVTQpG(Qα)) | |
| 267 | Cyclo-(Phe-4-ClHLETTFTGDGTPKTIRVTQpG(Qα)) | |
| 268 | Cyclo-(Phe-3-ClHLETTFTGDGTPKTIRVTQpG(Qα)) | |
| 269 | Cyclo-(Phe-2-ClHLETTFTGDGTPKTIRVTQpG(Qα)) | |
| 270 | Cyclo-(D-Phe-4- | |
| | NH₂HLETTFTGDGTPKTIRVTQpG(Qα)) | |
| 271 | Cyclo-(HomoPheHLETTFTGDGTPKTIRVTQpG(Qα)) | |
| 272 | Cyclo-(KHLETTFTGDGTPKTIRVTQpG(Qα)) | |
| 273 | Cyclo-(hHLETTFTGDGTPKTIRVTQpG(Qα)) | |
| 274 | Cyclo-(Phe-4- | |
| | NH₂HLETTFTGDGTPKTIRVTQpG(Qα)) | |
| 275 | Cyclo-(AlloThrHLETTFTGDGTPKTIRVTQpG(Qα)) | |
| 276 | Cyclo-(tHLETTFTGDGTPKTIRVTQpG(Qα)) | |
| 277 | Cyclo-(RHLETTFTGDGTPKTIRVTQpG(Qα)) | |
| 278 | Cyclo-(FHLETTFTGDGTPKTIRVTQpG(Qα)) | |
| 279 | Cyclo-(YHLETTFTGDGTPKTIRVTQpG(Qα)) | |
| 280 | Cyclo-(THLETT-WGDGHomoSerPKTIRVTQpG(Qα)) | |
| 281 | Cyclo- | |
| | (THLETTFWGDGHomoGlnPKTIRVTQpG(Qα)) | |
| 282 | Cyclo-(THLETTFWGDGGlu(OAll)PKTIRVTQpG(Qα)) | |
| 284 | Cyclo-(THLETTFWGDGePKTIRVTQpG(Qα)) | |
| 285 | Cyclo-(THLETTFWGDGDPKTIRVTQpG(QG)) | |
| 286 | Cyclo-(THLETTFWGDGdPKTIRVTQpG(Qα)) | |
| 287 | Cyclo-(THLETTFWGDGCys.AcidPKTIRVTQpG(Qα)) | |
| 288 | Cyclo-(THLETTFWGDGApiPKTIRVTQpG(Qα)) | |
| 289 | Cyclo-(THLETTFWGDGAlloThrPKTIRVTQpG(Qα)) | |
| 290 | Cyclo-(THLETTFYGDGEPKTIRVTQpG(Qα)) | |
| 291 | Cyclo-(THLETTFyGDGEPKTIRVTQpG(Qα)) | |
| 293 | Cyclo-(THLETTFwGDGEPKTIRVTQpG(Qα)) | |
| 294 | Cyclo- | |
| | (THLETTFNapthoic.AcidGDGEPKTIRVTQpG(Qα))c | |
| 295 | Cyclo-(THLETTFNal2GDGEPKTIRVTQpG(Qα)) | |
| 296 | Cyclo-(THLETTFNal1GDGEPKTIRVTQpG(Qα)) | |
| 297 | Cyclo-(THLETTFIndaGGDGEPKTIRVTQpG(Qα)) | |
| 298 | Cyclo-(THLETTFHomoChaGDGEPKTIRVTQpG(Qα)) | |
| 299 | Cyclo-(THLETTFPhe-4- | |
| | NH₂GDGEPKTIRVTQpG(Qα)) | |
| 300 | Cyclo-(THLETTFDapGDGEPKTIRVTQpG(Qα)) | |
| 301 | Cyclo-(THLETTFHomoPheGDGEPKTIRVTQpG(Qα)) | |
| 302 | Cyclo-(THLETTFHGDGEPKTIRVTQpG(Qα)) | |
| 303 | Cyclo-(THLETTFFGDGEPKTIRVTQpG(Qα)) | |
| 304 | Cyclo-(THLETTFfGDGEPKTIRVTQpG(Qα)) | |
| 305 | Cyclo-(THLETTFD- | |
| | HomoChaGDGEPKTIRVTQpG(Qα)) | |
| 306 | Cyclo-(THLETTFD-DapGDGEPKTIRVTQpG(Qα)) | |
| 307 | Cyclo-(THLETTFD-CitGDGEPKTIRVTQpG(Qα)) | |
| 308 | Cyclo-(THLETTFD-ChaGDGEPKTIRVTQpG(Qα)) | |
| 309 | Cyclo-(THLETTFD-Tyr(Et)GDGEPKTIRVTQpG(Qα)) | |
| 310 | Cyclo-(THLETTFCitGDGEPKTIRVTQpG(Qα)) | |
| 311 | Cyclo-(THLETTFChaGDGEPKTIRVTQpG(Qα)) | |
| 312 | Cyclo-(THLETTFBIPGDGEPKTIRVTQpG(Qα)) | |
| 313 | Cyclo-(THLETTFAlloThrGDGEPKTIRVTQpG(Qα)) | |
| 314 | Cyclo-(THLETTFAicGDGEPKTIRVTQpG(Qα)) | |
| 315 | Cyclo-(THLETTFPhe-2-MeGDGEPKTIRVTQpG(Qα)) | |
| 316 | Cyclo-(THLETTFD-Phe-2- | |
| | (3,4Cl)GDGEPKTIRVTQpG(Qα)) | |
| 317 | Cyclo-(THLETTFTrp-2-MeGDGEPKTIRVTQpG(Qα)) | |
| 318 | Cyclo-(THLETtFWGDGEPKTIRVTQpG(Qα)) | |
| 319 | Cyclo-(THLETEFWGDGEPKTIRVTQpG(Qα)) | |
| 320 | Cyclo-(THLETeFWGDGEPKTIRVTQpG(Qα)) | |
| 321 | Cyclo-(THLETDFWGDGEPKTIRVTQpG(Qα)) | |
| 322 | Cyclo-(THLETdFWGDGEPKTIRVTQpG(Qα)) | |
| 323 | Cyclo-(THLETCys.AcidFWGDGEPKTIRVTQpG(Qα)) | |
| 324 | Cyclo-(THLETApiFWGDGEPKTIRVTQpG(Qα)) | |
| 325 | Cyclo-(THLETAlloThrFWGDGEPKTIRVTQpG(Qα)) | |
| 326 | Cyclo-(THLEtTFWGDGEPKTIRVTQpG(Qα)) | |
| 327 | Cyclo-(THLEAlloThrTFWGDGEPKTIRVTQpG(Qα)) | |
| 328 | Cyclo-(YHLETTFWGDGEPKTIRVTQPG(Qα)) | |
| 329 | Cyclo-(tHLETTFWGDGEPKTIRVTQPG(Qα)) | |
| 330 | Cyclo-(RHLETTFWGDGEPKTIRVTQPG(Qα)) | |
| 331 | Cyclo-(Phe-4-ClHLETTFWGDGEPKTIRVTQpG(Qα)) | |
| 332 | Cyclo-(Phe-3-ClHLETTFWGDGEPKTIRVTQpG(Qα)) | |
| 333 | Cyclo-(Phe-2-ClHLETTFWGDGEPKTIRVTQpG(Qα)) | |
| 334 | Cyclo-(Phe-4- | |
| | NH₂HLETTFWGDGEPKTIRVTQpG(Qα)) | |
| 335 | Cyclo-(KHLETTFWGDGEPKTIRVTQPG(Qα)) | |
| 336 | Cyclo- | |
| | (HomoPheHLETTFWGDGEPKTIRVTQpG(Qα)) | |
| 337 | Cyclo-(hHLETTFWGDGEPKTIRVTQPG(Qα)) | |
| 338 | Cyclo-(FHLETTFWGDGEPKTIRVTQPG(Qα)) | |
| 339 | Cyclo-(D-Phe-4- | |
| | NH₂HLETTFWGDGEPKTIRVTQpG(Qα)) | |
| 340 | Cyclo-(AlloThrHLETTFWGDGEPKTIRVTQpG(Qα)) | |
| 341 | Cyclo-(THVETTFWGDGEPKTIRVTQPG(Qα)) | |
| 342 | Cyclo-(THvETTFWGDGEPKTIRVTQpG(Qα)) | |
| 343 | Cyclo-(THTleETTFWGDGEPKTIRVTQpG(Qα)) | |
| 344 | Cyclo-(THNValETTFWGDGEPKTIRVTQPG(Qα)) | |
| 345 | Cyclo-(THNLeuETTFWGDGEPKTIRVTQPG(Qα)) | |
| 346 | Cyclo-(THIETTFWGDGEPKTIRVTQPG(Qα)) | |
| 347 | Cyclo- | |
| | (THHomoLeuETT-WGDGEPKTIRVTQPG(Qα)) | |
| 348 | Cyclo- | |
| | (THHomoChaETT-WGDGEPKTIRVTQPG(Qα)) | |
| 349 | Cyclo-(THD-OrnETTFWGDGEPKTIRVTQPG(Qα)) | |
| 350 | Cyclo-(THD- | |
| | HomoChaETTFWGDGEPKTIRVTQPG(Qα)) | |
| 351 | Cyclo-(THCycLeuETTFWGDGEPKTIRVTQPG(Qα)) | |
| 352 | Cyclo-(THChaETTFWGDGEPKTIRVTQPG(Qα)) | |
| 353 | Cyclo-(THAibETTFWGDGEPKTIRVTQPG(Qα)) | |
| 354 | Cyclo-(THAbuETTFWGDGEPKTIRVTQPG(QG)) | |
| 355 | Cyclo-(TYLETTFWGDGEPKTIRVTQPG(Qα)) | |
| 356 | Cyclo-(TThiLETTFWGDGEPKTIRVTQpG(Qα)) | |
| 357 | Cyclo-(TRLETTFWGDGEPKTIRVTQPG(Qα)) | |
| 358 | Cyclo-(TOrnLETTFWGDGEPKTIRVTQpG(Qα)) | |
| 359 | Cyclo-(TPhe-4- | |
| | NH₂LETFWGDGEPKTIRVTQpG(Qα)) | |
| 360 | Cyclo-(TKLETTFWGDGEPKTIRVTQPG(Qα)) | |
| 361 | Cyclo-(TKLETTFWGDGEPKTIRVTQPG(Qα)) | |
| 362 | Cyclo- | |
| | (THomoLysLETTFWGDGEPKTIRVTQpG(Qα)) | |
| 363 | Cyclo-(THomoCitLETTFWGDGEPKTIRVTQpG(Qα)) | |
| 364 | Cyclo- | |
| | (THomoArgLETTFWGDGEPKTIRVTQpG(Qα)) | |
| 365 | Cyclo-(THis(3-Me)LETTFWGDGEPKTIRVTQPG(Qα)) | |
| 366 | Cyclo-(THis(1-Me)LETTFWGDGEPKTIRVTQPG(Qα)) | |
| 367 | Cyclo-(TFurAlaLETTFWGDGEPKTIRVTQpG(Qα)) | |
| 368 | Cyclo-(TPyA-4LETTFWGDGEPKTIRVTQpG(Qα)) | |
| 369 | Cyclo-(TPhe-4-N3LETTFWGDGEPKTIRVTQPG(Qα)) | |
| 370 | Cyclo-(THLETTFWGDR(Me2)EPKTIRVTQpG(Qα)) | |
| 371 | Cyclo-(TPhe-4- | |
| | CH₂N₂LETTFWGDGEPKTIRVTQPG(Qα)) | |
| 372 | Cyclo-(THLETTFWGDR(Me)EPKTIRVTQpG(Qα)) | |
| 373 | Cyclo-(THLETTFWGDREPKTIRVTQpG(Qα)) | |
| 374 | Cyclo-(THLETTFWGDPEPKTIRVTQPG(Qα)) | |
| 375 | Cyclo-(THLETTFWGDpEPKTIRVTQpG(Qα)) | |
| 376 | Cyclo-(THLETTFWGDAEPKTIRVTQpG(Qα)) | |
| 377 | Cyclo-(THLETTFWGDaEPKTIRVTQpG(Qα)) | |
| 378 | Cyclo-(THLETTFWHomoGlnDGEPKTIRVTQPG(Qα)) | |
| 379 | Cyclo-(THLETTFWHomoCitDGEPKTIRVTQPG(Qα)) | |
| 380 | Cyclo-(THLETTFWD-CitDGEPKTIRVTOpG(Qα)) | |
| 381 | Cyclo-(THLETTFWCitDGEPKTIRVTQpG(Qα)) | |
| 382 | Cyclo-(THLETTStyAlaWGDGEPKTIRVTQpG(Qα)) | |
| 383 | Cyclo-(THLETTPhgWGDGEPKTIRVTQpG(Qα)) | |
| 384 | Cyclo-(THLETTPhe-4-FWGDGEPKTIRVTQpG(Qα)) | |
| 385 | Cyclo-(THLETTPhe-4-ClWGDGEPKTIRVTQpG(Qα)) | |
| 386 | Cyclo-(THLETTPhe-4-BrWGDGEPKTIRVTQpG(Qα)) | |
| 387 | Cyclo-(THLETTPhe-3-FWGDGEPKTIRVTQpG(Qα)) | |
| 388 | Cyclo-(THLETTPhe-3-ClWGDGEPKTIRVTQpG(Qα)) | |
| 389 | Cyclo-(THLETTPhe-3-BrWGDGEPKTIRVTQpG(Qα)) | |
| 390 | Cyclo-(THLETTPhe-2-FWGDGEPKTIRVTQpG(Qα)) | |
| 391 | Cyclo-(THLETTPhe-2-ClWGDGEPKTIRVTQpG(Qα)) | |
| 392 | Cyclo-(THLETTPhe-2-BrWGDGEPKTIRVTQpG(Qα)) | |
| 393 | Cyclo-(THLETTNal2WGDGEPKTIRVTQpG(Qα)) | |
| 394 | Cyclo-(THLETTNal1WGDGEPKTIRVTQpG(Qα)) | |
| 395 | Cyclo- | |
| | (THLETTHomoChaWGDGEPKTIRVTQpG(Qα)) | |
| 396 | Cyclo- | |
| | (THLETTHomoPheWGDGEPKTIRVTQpG(Qα)) | |
| 397 | Cyclo-(THLETTPhe- | |
| | 4 [symbol] Bu)WGDGEPKTIRVTQpG(Qα)) | |
| 398 | Cyclo-(THLETTfWGDGEPKTIRVTQpG(Qα)) | |
| 399 | Cyclo-(THLETTD- | |
| | HomoChaWGDGEPKTIRVTQpG(Qα)) | |
| 400 | Cyclo-(THLETTD-ChaWGDGEPKTIRVTQpG(Qα)) | |
| 401 | Cyclo-(THLETTChaWGDGEPKTIRVTQpG(Qα)) | |
| 402 | Cyclo-(THLETTBIPWGDGEPKTIRVTQpG(Qα)) | |
| 403 | Cyclo-(THLETTPhe-4- | |
| | MeWGDGEPKTIRVTQpG(Qα)) | |
| 404 | Cyclo-(THLETTPhe-3- | |
| | MeWGDGEPKTIRVTQpG(Qα)) | |
| 405 | Cyclo-(THLETTPhe-2- | |
| | MeWGDGEPKTIRVTQpG(Qα)) | |
| 406 | Cyclo-(THLRTTFWGDGEPKTIRVTQPG(Qα)) | |
| 407 | Cyclo-(THLQTTFWGDGEPKTIRVTQPG(Qα)) | |
| 408 | Cyclo-(THLOrnTTFWGDGEPKTIRVTOpG(Qα)) | |
| 409 | Cyclo-(THLHomoSerTTFWGDGEPKTIRVTQPG(Qα)) | |
| 410 | Cyclo- | |
| | (THLHomoGlnTTFWGDGEPKTIRVTQpG(Qα)) | |
| 411 | Cyclo-(THLGlu(OAll)TTFWGDGEPKTIRVTQPG(Qα)) | |
| 412 | Cyclo-(THLeTTFWGDGEPKTIRVTQpG(Qα)) | |
| 413 | Cyclo-(THLDTTFWGDGEPKTIRVTQPG(Qα)) | |
| 414 | Cyclo-(THLdTTFWGDGEPKTIRVTQpG(Qα)) | |
| 415 | Cyclo- | |
| | (THLCysteic.AcidTTFWGDGEPKTIRVTQpG(Qα)) | |
| 416 | Cyclo-(THLETTFWGEGEPKTIRVTQpG(Qα)) | |
| 417 | Cyclo-(THLdTTFWGeGEPKTIRVTQpG(Qα)) | |
| 418 | Cyclo-(THLdTTFWGdGEPKTIRVTQPG(Qα)) | |
| 419 | THLETTFWGDGEPKTIRVTQ | |
| 420 | THLETTFGDGEPKTIRVTQ | |
| 421 | TH(3-Me)LETTFWGDGEPKTIRVTQ | |
| 422 | TH(3-Me)LETTFGDGEPKTIRVTQ | |
| 423 | cyclo-(THLETTFWGDGEPKTIRVTQpG(Eα)) | |
| 424 | cyclo-(THLETTFGDGEPKTIRVTQpG(Eα)) | |
| 425 | cyclo-(TH(3-Me)LETTFWGDGEPKTIRVTQPG(Eα)) | |
| 426 | cyclo-(TH(3-Me)LETTFGDGEPKTIRVTQpG(Eα)) | |
| 427 | cyclo-(THLETTFTGDGTP(K-CA)TIRVTQpGQ) | K-CA = lysine |
| | | modified with |
| | | chloroalkane-tag |
| 428 | cyclo-(CTHLETTFTGDGTPKTIRVTQpG) | |
| 429 | THLETTFTGDGTPKTIRVTQ | |
| 430 | cyclo-(Ac¹⁰⁵THLETTFTGDGTPKTIRVTQ¹²⁴pGC) | |
| 431 | cyclo-(¹⁰⁵THLETTFTGDGTPKTIRVTQ¹²⁴pG(Eγ)) | |
| 432 | cyclo-(105THLETTFTGDGTPKTIRVTQ124pG(Dγ)) | |
| 433 | cyclo-(¹⁰⁵THLETTFTGDGTPKTIRVTQ¹²⁴pG(Eα))* | |
| 434 | LETTF | |
| 435 | KLSSIESDV | |
| 436 | LETX₂X₃X₄GX₅X₆X₇ | X₂ is T, S, D or E; |
| | | X₃ is F, F-2-Br, F-2-Cl |
| | | or F-3-F; |
| | | X₄ is W, Nal, or |
| | | absent; |
| | | X₅ is D or N-Me-D; |
| | | X₆ is G, A or P; and |
| | | X₇ is E or D; |
| 437 | CTGATCGCGGGCCCGAA | Primer |
| 438 | TTTGATTTCCATCACTTTTTCCGC | Primer |
| 439 | CCCGGCGGGCCTGGGC | Primer |
| 440 | CCTTTGATCAGTTTGATTTCC | Primer |
| 441 | GTGACCGCGATTATTGAAGGCG | Primer |
| 442 | ATAAATGCTGTTATCGCCCGG | Primer |
| 443 | GATGTGATGGCGGAAGATGC | Primer |
| 444 | TTCCAGGCCCACGCTGTTC | Primer |
| 445 | GCGCTGGCGAACACCTATGA | Primer |
| 446 | CGCCACCGCATCTTCATGCA | Primer |
| 447 | CTGGGCATTAGCATTGCGGG | Primer |
| 448 | GCCTTTCGGGCCTTTGATCAG | Primer |
| 449 | GCTTTGCGATTGCGGGCGGT | Primer |
| 450 | CCAGGCCTTTCGGGCCTTTGAT | Primer |
| 451 | CATTTATGTGGCGAAAATTATTGAAGG-CGGTGC | Primer |
| 452 | CTGTTATCGCCCGGAATATGCTGGTTG | Primer |
| 453 | CATGAAGATGCGGCAGCGGCG | Primer |
| 454 | CATCACATCTTCCAGGCCCACGC | Primer |
| 455 | GGTGTGGGCGCGCAGCATATTC | Primer |
| 456 | GCCCGCAATGCTAAAGCCCAGGC | Primer |
| 457 | GTGATGCATGCGGATGCGGTG | Primer |
| 458 | ATCTTCCAGGCCCACGCTGTTC | Primer |
| 459 | CTGGGCGCGAGCATTGCGGG | Primer |
| 460 | GCCTTTCGGGCCTTTGATCAG | Primer |

### REFERENCES

1. Faden, A., Demediuk, P., Panter, S. & Vink, R. The role of excitatory amino acids and NMDA receptors in traumatic brain injury. Science 244, 798-800 (1989).

2. Sattler, R. & Tymianski, M. Molecular mechanisms of glutamate receptor-mediated excitotoxic neuronal cell death. Mol. Neurobiol. 24, 107-129 (2001).

3. Mehta, S. L., Manhas, N. & Raghubir, R. Molecular targets in cerebral ischemia for developing novel therapeutics. Brain. Res. Rev. 54, 34-66 (2007).

4. Ji, H.; Li, H.; Martasek, P.; Roman, L. J.; Poulos, T. L.; Silverman, R. B., Discovery of highly potent and selective inhibitors of neuronal nitric oxide synthase by fragment hopping. J. Med. Chem. 2009, 52 (3), 779-797.

5. Ji, H.; Tan, S.; Igarashi, J.; Li, H.; Derrick, M.; Martasek, P.; Roman, L. J.; Vasquez-Vivar, J.; Poulos, T. L.; Silverman, R. B., Selective neuronal nitric oxide synthase inhibitors and the prevention of cerebral palsy. Ann. Neurol. 2009, 65 (2), 209-217.

6. Silverman, R. B., Design of selective neuronal nitric oxide synthase inhibitors for the prevention and treatment of neurodegenerative diseases. Acc. Chem. Res. 2009, 42 (3), 439-451.

7. Diener, H. C.; AlKhedr, A.; Busse, O.; Hacke, W.; Zingmark, P. H.; Jonsson, N.; Basun, H., Treatment of acute ischaemic stroke with the low-affinity, use-dependent NMDA antagonist AR-R15896AR. J. Neurol. 2002, 249 (5), 561-568.

8. Hardingham, G. E.; Bading, H., The Yin and Yang of NMDA receptor signalling. Trends Neurosci. 2003, 26 (2), 81-89.

9. Hoyte, L.; Barber, P. A.; Buchan, A. M.; Hill, M. D., The rise and fall of NMDA antagonists for ischemic stroke. Curr. Mol. Med. 2004, 4 (2), 131-136.

10. Milani, D.; Cross, J. L.; Anderton, R. S.; Blacker, D. J.; Knuckey, N. W.; Meloni, B. P., Neuroprotective efficacy of poly-arginine R18 and NA-1 (TAT-NR2B9c) peptides following transient middle cerebral artery occlusion in the rat. Neurosci Res 2017, 114, 9-15.

11. Hill, M. D.; Goyal, M.; Menon, B. K.; Nogueira, R. G.; McTaggart, R. A.; Demchuk, A. M.; Poppe, A. Y.; Buck, B. H.; Field, T. S.; Dowlatshahi, D., Efficacy and safety of nerinetide for the treatment of acute ischaemic stroke (ESCAPE-NA1): a multicentre, double-blind, randomised controlled trial. Lancet 2020, 395 (10227), 878-887.

12. Bach, A. et al. Design and synthesis of highly potent and plasma-stable dimeric inhibitors of the PSD-95-NMDA receptor interaction. Angew. Chem. Int. Ed. 2009, 48 (51), 9685-9689.

13. Bach, A. et al. A high-affinity, dimeric inhibitor of PSD-95 bivalently interacts with PDZ1-2 and protects against ischemic brain damage. Proc. Natl. Acad. Sci. U.S.A. 109, 3317-3322 (2012).

14. Aarts, M.; Liu, Y.; Liu, L.; Besshoh, S.; Arundine, M.; Gurd, J. W.; Wang, Y. T.; Salter, M. W.; Tymianski, M., Treatment of ischemic brain damage by perturbing NMDA receptor-PSD-95 protein interactions. Science 2002, 298 (5594), 846-850.

15. Qu, W.; Liu, N. K.; Wu, X.; Wang, Y.; Xia, Y.; Sun, Y.; Lai, Y.; Li, R.; Shekhar, A.; Xu, X. M., Disrupting nNOS-PSD95 interaction improves neurological and cognitive recoveries after traumatic brain injury. Cereb. Cortex. 2020.

16. Oschkinat, H., A new type of PDZ domain recognition. Nat. Struct. Biol. 1999, 6 (5), 408-410.

17. Songyang, Z.; Fanning, A. S.; Fu, C.; Xu, J.; Marfatia, S. M.; Chishti, A. H.; Crompton, A.; Chan, A. C.; Anderson, J. M.; Cantley, L. C., Recognition of unique carboxyl-terminal motifs by distinct PDZ domains. Science 1997, 275 (5296), 73-77.

18. Bezprozvanny, I.; Maximov, A., Classification of PDZ domains. FEBS Lett. 2001, 509 (3), 457-462.

19. Tonikian, R.; Zhang, Y.; Sazinsky, S. L.; Currell, B.; Yeh, J. H.; Reva, B.; Held, H. A.; Appleton, B. A.; Evangelista, M.; Wu, Y.; Xin, X.; Chan, A. C.; Seshagiri, S.; Lasky, L. A.; Sander, C.; Boone, C.; Bader, G. D.; Sidhu, S. S., A specificity map for the PDZ domain family. PLoS Biol 2008, 6 (9), e239.

20. Kalyoncu, S.; Keskin, O.; Gursoy, A., Interaction prediction and classification of PDZ domains. BMC Bioinform. 2010, 11, 357.

21. Lim, I. A.; Hall, D. D.; Hell, J. W., Selectivity and promiscuity of the first and second PDZ domains of PSD-95 and synapse-associated protein 102. J. Biol. Chem. 2002, 277 (24), 21697-21711.

22. Skelton, N. J.; Koehler, M. F.; Zobel, K.; Wong, W. L.; Yeh, S.; Pisabarro, M. T.; Yin, J. P.; Lasky, L. A.; Sidhu, S. S., Origins of PDZ domain ligand specificity.

Structure determination and mutagenesis of the Erbin PDZ domain. J. Biol. Chem. 2003, 278 (9), 7645-7654.

23. Johnson-Leger, C.; Power, C. A.; Shomade, G.; Shaw, J. P.; Proudfoot, A. E., Protein therapeutics-lessons learned and a view of the future. Expert. Opin. Biol. Ther. 2006, 6 (1), 1-7.

24. Antosova, Z.; Mackova, M.; Kral, V.; Macek, T., Therapeutic application of peptides and proteins: parenteral forever? Trends Biotechnol. 2009, 27 (11), 628-635.

25. Robinson, J. A., β-Hairpin peptidomimetics: design, structures and biological activities. Acc. Chem. Res. 2008, 41 (10), 1278-1288.

26. Jha, A.; Kumar, M. G.; Gopi, H. N.; Paknikar, K. M., Inhibition of beta-Amyloid Aggregation through a Designed beta-Hairpin Peptide. Langmuir 2018, 34 (4), 1591-1600.

27. Cho, P. Y.; Joshi, G.; Boersma, M. D.; Johnson, J. A.; Murphy, R. M., A Cyclic peptide mimic of the beta-amyloid binding domain on transthyretin. ACS Chem. Neurosci. 2015, 6 (5), 778-789.

28. Danelius, E.; Pettersson, M.; Bred, M.; Min, J.; Waddell, M. B.; Guy, R. K.; Grotli, M.; Erdelyi, M., Flexibility is important for inhibition of the MDM2/p53 protein-protein interaction by cyclic beta-hairpins. Org. Biomol. Chem. 2016, 14 (44), 10386-10393.

29. DeMarco, S. J.; Henze, H.; Lederer, A.; Moehle, K.; Mukherjee, R.; Romagnoli, B.; Robinson, J. A.; Brianza, F.; Gombert, F. O.; Lociuro, S., Discovery of novel, highly potent and selective β-hairpin mimetic CXCR4 inhibitors with excellent anti-HIV activity and pharmacokinetic profiles. Bioorg. Med. Chem. 2006, 14 (24), 8396-8404.

30. Lesniak, W. G.; Sikorska, E.; Shallal, H.; Behnam Azad, B.; Lisok, A.; Pullambhatla, M.; Pomper, M. G.; Nimmagadda, S., Structural characterization and in vivo evaluation of beta-hairpin peptidomimetics as specific CXCR4 imaging agents. Mol Pharm. 2015, 12 (3), 941-953.

31. Gee, S. H.; Sekely, S. A.; Lombardo, C.; Kurakin, A.; Froehner, S. C.; Kay, B. K., Cyclic peptides as non-carboxyl-terminal ligands of syntrophin PDZ domains. J. Biol. Chem. 1998, 273 (34), 21980-21987.

32. LeBlanc, B. W.; Iwata, M.; Mallon, A. P.; Rupasinghe, C. N.; Goebel, D. J.; Marshall, J.; Spaller, M. R.; Saab, C. Y., A cyclic peptide targeted against PSD-95 blocks central sensitization and attenuates thermal hyperalgesia. Neuroscience 2010, 167 (2), 490-500.

33. Piserchio, A.; Salinas, G. D.; Li, T.; Marshall, J.; Spaller, M. R.; Mierke, D. F., Targeting specific PDZ domains of PSD-95. ACS Chem. Biol. 2004, 11 (4), 469-473.

34. Udugamasooriya, G.; Saro, D.; Spaller, M. R., Bridged peptide macrocycles as ligands for PDZ domain proteins. Org. Lett. 2005, 7 (7), 1203-1206.

35. Hammond, M. C.; Harris, B. Z.; Lim, W. A.; Bartlett, P. A., Beta strand peptidomimetics as potent PDZ domain ligands. Chem. Biol. 2006, 13 (12), 1247-1251.

36. Hoppmann, C.; Seedorff, S.; Richter, A.; Fabian, H.; Schmieder, P.; Ruck-Braun, K.; Beyermann, M., Light-directed protein binding of a biologically relevant β-sheet. Angew. Chem. Int. Ed. 2009, 48 (36), 6636-6639.

37. Seedorff, S.; Appelt, C.; Beyermann, M.; Schmieder, P., Design, synthesis, structure and binding properties of PDZ binding, cyclic beta-finger peptides. Biochem. Biophys. Res. Commun. 2010, 395 (4), 535-539.

38. Pedersen, S. W.; Pedersen, S. B.; Anker, L.; Hultqvist, G.; Kristensen, A. S.; Jemth, P.; Strømgaard, K., Probing backbone hydrogen bonding in PDZ/ligand interactions by protein amide-to-ester mutations. Nat. Commun. 2014, 5, 3215

39. Pedersen, S. W.; Moran, G. E.; Sereikaite, V.; Haugaard-Kedström, L. M.; Strømgaard, K., mportance of a conserved Lys/Arg residue for ligand/PDZ domain interactions as examined by protein semisynthesis. Chembiochem 2016, 17 (20), 1936-1944.

40. Nikolovska-Coleska, Z.; Wang, R.; Fang, X.; Pan, H.; Tomita, Y.; Li, P.; Roller, P. P.; Krajewski, K.; Saito, N. G.; Stuckey, J. A., Development and optimization of a binding assay for the XIAP BIR3 domain using fluorescence polarization. Anal. Biochem. 2004, 332 (2), 261-273.

41. Nikolovska-Coleska, Z.; Meagher, J. L.; Jiang, S.; Kawamoto, S. A.; Gao, W.; Yi, H.; Qin, D.; Roller, P. P.; Stuckey, J. A.; Wang, S., Design and characterization of bivalent Smac-based peptides as antagonists of XIAP and development and validation of a fluorescence polarization assay for XIAP containing both BIR2 and BIR3 domains. Anal. Biochem. 2008, 374 (1), 87-98.

42. Winn, M.; Ballard, C.; Cowtan, K.; Dodson, E.; Emsley, P.; Evans, P.; Keegan, R.; Krissinel, E.; Leslie, A.; McCoy, A., Overview of the CCP4 suite and current developments. Acta Crystallogr. Sect. D, Biol. Crystallogr. 2011, 67 (4), 235-242.

43. McCoy, A. J.; Grosse-Kunstleve, R. W.; Adams, P. D.; Winn, M. D.; Storoni, L. C.; Read, R. J., Phaser crystallographic software. J. Appl. Crystallogr. 2007, 40 (4), 658-674.

44. Hillier, B. J., Unexpected modes of PDZ domain scaffolding revealed by structure of nNOS-Syntrophin complex. Science 1999, 284 (5415), 812-815.

45. Pettersen, E. F.; Goddard, T. D.; Huang, C. C.; Couch, G. S.; Greenblatt, D. M.; Meng, E. C.; Ferrin, T. E., UCSF Chimera-a visualization system for exploratory research and analysis. J. Comput. Chem. 2004, 25 (13), 1605-1612.

46. Sainlos, M.; Tigaret, C.; Poujol, C.; Olivier, N. B.; Bard, L.; Breillat, C.; Thiolon, K.; Choquet, D.; Imperiali, B., Biomimetic divalent ligands for the acute disruption of synaptic AM PAR stabilization. Nat. Chem. Biol 2011, 7 (2), 81.

47. Harris, B. Z.; Hillier, B. J.; Lim, W. A., Energetic determinants of internal motif recognition by PDZ domains. Biochem. J. 2001, 40 (20), 5921-5930.

48. Walma, T. et al. Structure, dynamics and binding characteristics of the second PDZ domain of PTP-BL. J. Mol. Biol. 316, 1101-1110 (2002).

49. Madsen, K. et al. Molecular determinants for the complex binding specificity of the PDZ domain in PICK1. J. Biol. Chem. 280, 20539-20548 (2005).

50. Velthuis, A., Sakalis, P., Fowler, D. & Bagowski, C. Genome-wide analysis of PDZ domain binding reveals inherent functional overlap within the PDZ interaction network. PLoS One 6, e16047 (2011).

51. Carson, M., Johnson, D., McDonald, H., Brouillette, C. & DeLucas, L. His-tag impact on structure. Acta Crystallogr. Sect. D, Biol. Crystallogr. 63, 295-301 (2007).

52. Zhang, J., Petit, C., King, D. & Lee, A. Phosphorylation of a PDZ domain extension modulates binding affinity and interdomain interactions in postsynaptic density-95 (PSD-95) protein, a membrane-associated guanylate kinase (MAGUK). J. Biol. Chem. 286, 41776-41785 (2011).

53. Pedersen, S. et al. Site-specific phosphorylation of PSD-95 PDZ domains reveals fine-tuned regulation of protein-protein interactions. ACS Chem. Biol. 12, 2313-2323 (2017).

54. Garcia, P. et al. Phosphorylation of synthetic peptides containing Tyr-Met-X-Met motifs by nonreceptor tyrosine kinases in vitro. J. Biol. Chem. 268, 25146-25151 (1993).

55. Blom, N.; Gammeltoft, S.; Brunak, S., Sequence and structure-based prediction of eukaryotic protein phosphorylation sites. J. Mol. Bio. 1999, 294 (5), 1351-1362.

56. Blom, N.; Sicheritz-Pontén, T.; Gupta, R.; Gammeltoft, S.; Brunak, S., Prediction of post-translational glycosylation and phosphorylation of proteins from the amino acid sequence. Proteomics 2004, 4 (6), 1633-1649.

57. Hornbeck, P. et al. PhosphoSitePlus: a comprehensive resource for investigating the structure and function of experimentally determined post-translational modifications in man and mouse. Nucleic Acids Res. 40, D261-D270 (2012).

58. Gnad, F., Gunawardena, J. & Mann, M. PHOSIDA 2011: the posttranslational modification database. Nucleic Acids Res. 39, D253-D260 (2010).

59. Nyman, T. et al. The role of MeH73 in actin polymerization and ATP hydrolysis. J. Mol. Biol. 317, 577-589 (2002).

60. Webb, K. J. et al. A novel 3-methylhistidine modification of yeast ribosomal protein Rpl3 is dependent upon the YIL110W methyltransferase. J. Biol. Chem. 285, 37598-37606 (2010).

61. Beuming, T., Farid, R. & Sherman, W. High-energy water sites determine peptide binding affinity and specificity of PDZ domains. Protein Sci. 18, 1609-1619 (2009).

62. Lindsay, M. et al. World Stroke Organization (WSO): Global stroke fact sheet 2019. Int. J. Stroke 14, 806-817 (2019).

63. Soriano, F. X. et al. Specific targeting of pro-death NMDA receptor signals with differing reliance on the NR2B PDZ ligand. J. Neurosci. Res. 28, 10696-10710 (2008).

1. A polypeptide comprising the amino acid sequence of
(SEQ ID NO: 1)
TX₁LETX₂X₃X₄GX₅X₆X₇PX₈TIRVX9Q
wherein
X₁ is H, H-3Me or PyA-4;
X₂ is T, S, D or E;
X₃ is F, F-2-Br, F-2-Cl or F-3-F;
X₄ is W, NaI, or absent;
X₅ is D or N-Me-D;
X₆ is G, A or P;
X₇ is E or D;
X₈ is K or N-Me-K; and
X₉ is T or N-Me-T;
or a pharmaceutically acceptable salt thereof.

2. The polypeptide according to claim 1, wherein the polypeptide is covalently linked to a cyclization moiety.

3. The polypeptide according to any one of the preceding claims, wherein the cyclization moiety comprises the amino acid sequence of pGX₁₀, wherein X₁₀ is C, Q or E.

4. The polypeptide according to any of the preceding claims, wherein the polypeptide comprises or consists of the amino acid sequence
(SEQ ID NO: 2)
TX₁LETX₂X₃X₄GX₅X₆X₇PX₈TIRVX₉QpGX₁₀
wherein
X₁ is H, H-3Me or PyA-4;
X₂ is T, S, D or E;
X₃ is F, F-2-Br, F-2-Cl or F-3-F;
X₄ is W, NaI, or absent;
X₅ is D or N-Me-D;
X₆ is G, A or P;
X₇ is E or D;
X₈ is K or N-Me-K;
X₉ is T or N-Me-T; and
X₁₀ is C, Q or E;
or a pharmaceutically acceptable salt thereof.

5. The polypeptide according to any of claims 1 to 3, wherein the polypeptide comprises or consists of the amino acid sequence
(SEQ ID NO: 3)
TX₁LETX₂X₃GX₅X₆X₇PX₈TIRVX₉Q
wherein
X₁ is H, H-3Me or PyA-4;
X₂ is T, S, D or E;
X₃ is F, F-2-Br, F-2-Cl or F-3-F;
X₅ is D or N-Me-D;
X₆ is G, A or P;
X₇ is E or D;
X₈ is K or N-Me-K; and
X₉ is T or N-Me-T;
or a pharmaceutically acceptable salt thereof.

6. The polypeptide according to claim 1, wherein the polypeptide comprises or consists of the amino acid sequence TX₁LETX₂X₃GX₅X₆X₇PX₈TIRVX₉QpGX₁₀ (SEQ ID NO: 4)
wherein
X₁ is H, H-3Me or PyA-4;
X₂ is T, S, D or E;
X₃ is F, F-2-Br, F-2-Cl or F-3-F;
X₅ is D or N-Me-D;
X₆ is G, A or P;
X₇ is E or D;
X₈ is K or N-Me-K;
X₉ is T or N-Me-T; and
X₁₀ is C, Q or E;
or a pharmaceutically acceptable salt thereof.

7. The polypeptide according to claim 1, wherein the polypeptide comprises or consists of the amino acid sequence TX₁LETTFX₄GX₅X₆X₇PX₈TIRVX₉QpGX₁₀ (SEQ ID NO: 5)
wherein
X₁ is H, H-3Me or PyA-4;
X₄ is W, NaI, or absent;
X₅ is D or N-Me-D;
X₆ is G, A or P;
X₇ is E or D;
X₈ is K or N-Me-K;
X₉ is T or N-Me-T; and
X₁₀ is C, Q or E;
or a pharmaceutically acceptable salt thereof.

8. The polypeptide according to claim 1, wherein the polypeptide comprises or consists of the amino acid sequence TX₁LETTFGX₅X₆X₇PX₈TIRVX₉QpGX₁₀ (SEQ ID NO: 6)
wherein
X₁ is H, H-3Me or PyA-4;
X₅ is D or N-Me-D;
X₆ is G, A or P;
X₇ is E or D;
X₈ is K or N-Me-K;
X₉ is T or N-Me-T; and
X₁₀ is C, Q or E;
or a pharmaceutically acceptable salt thereof.

9. The polypeptide according to claim 1, wherein the polypeptide comprises or consists of the amino acid sequence TX₁LETTFX₄GDGX₇PX₈TIRVX₉Q (SEQ ID NO: 7), wherein
X₁ is H, or PyA-4;
X₄ is W or NaI;
X₇ is E or D;
X₈ is K or N-Me-K; and
X₉ is T or N-Me-T;
or a pharmaceutically acceptable salt thereof.

10. The polypeptide according to any of the preceding claims, wherein said polypeptide is cyclic.

11. The polypeptide according to any of the preceding claims, wherein said polypeptide is back-bone cyclized.

12. The polypeptide according to any of the preceding claims, wherein X₁ is H.

13. The polypeptide according to any of the preceding claims, wherein X₁ is H-3Me.

14. The polypeptide according to any of the preceding claims, wherein X₁ is PyA-4.

15. The polypeptide according to any of the preceding claims, wherein X₂ is T.

16. The polypeptide according to any of the preceding claims, wherein X₂ is S.

17. The polypeptide according to any of the preceding claims, wherein X₂ is D.

18. The polypeptide according to any of the preceding claims, wherein X₂ is E.

19. The polypeptide according to any of the preceding claims, wherein X₃ is F.

20. The polypeptide according to any of the preceding claims, wherein X₃ is F-2-Br.

21. The polypeptide according to any of the preceding claims, wherein X₃ is F-2-Cl.

22. The polypeptide according to any of the preceding claims, wherein X₃ is F-3-F.

23. The polypeptide according to any of the preceding claims, wherein X₄ is W.

24. The polypeptide according to any of the preceding claims, wherein X₄ is NaI.

25. The polypeptide according to any of the preceding claims, wherein X₅ is D.

26. The polypeptide according to any of the preceding claims, wherein X₅ is N-Me-D.

27. The polypeptide according to any of the preceding claims, wherein X₆ is G.

28. The polypeptide according to any of the preceding claims, wherein X₆ is A.

29. The polypeptide according to any of the preceding claims, wherein X₆ is P.

30. The polypeptide according to any of the preceding claims, wherein X₇ is E.

31. The polypeptide according to any of the preceding claims, wherein X₇ is D.

32. The polypeptide according to any of the preceding claims, wherein X₈ is K.

33. The polypeptide according to any of the preceding claims, wherein X₈ is N-Me-K.

34. The polypeptide according to any of the preceding claims, wherein X₉ is T.

35. The polypeptide according to any of the preceding claims, wherein X₉ is N-Me-T.

36. The polypeptide according to any of the preceding claims, wherein X₁₀ is C.

37. The polypeptide according to any of the preceding claims, wherein X₁₀ is Q.

38. The polypeptide according to any of the preceding claims, wherein X₁₀ is E.

39. The polypeptide according to any of the preceding claims, wherein X₁ is H, X₂ is T, X₃ is F, X₄ is W, X₅ is D, and X₆ is G.

40. The polypeptide according to any of the preceding claims, wherein X₁ is H, X₂ is T, X₃ is F, X₄ is W, X₅ is D, X₆ is G, and X₇ is E.

41. The polypeptide according to any of the preceding claims, wherein X₁ is H, X₂ is T, X₃ is F, X₄ is W, X₅ is D, X₆ is G, and X₇ is D.

42. The polypeptide according to any of the preceding claims, wherein X₁ is H, X₂ is T, X₃ is F, X₄ is NaI, X₅ is D, X₆ is G, and X₇ is E.

43. The polypeptide according to any of the preceding claims, wherein X₁ is H, X₂ is PyA-4, X₃ is F, X₄ is W, X₅ is D, X₆ is G, and X₇ is E.

44. The polypeptide according to any of the preceding claims, wherein the polypeptide comprises an amino acid sequence selected from the group consisting of:
(SEQ ID NO: 8)
THLETTFWGDGE,
(SEQ ID NO: 9)
THLETTFWGDGD,
(SEQ ID NO: 10)
THLETTF(Nal)GDGE,
and
(SEQ ID NO: 11)
T(PyA-4)LETTFWGDGE.

45. The polypeptide according to any of the preceding claims, wherein the polypeptide comprises the amino acid sequence THLETTFWGDGE (SEQ ID NO: 8).

46. The polypeptide according to any of the preceding claims, wherein the polypeptide comprises the amino acid sequence THLETTFWGDGD (SEQ ID NO: 9).

47. The polypeptide according to any of the preceding claims, wherein the polypeptide comprises the amino acid sequence THLETTF(NaI)GDGE (SEQ ID NO: 10).

48. The polypeptide according to any of the preceding claims, wherein the polypeptide comprises the amino acid sequence T(PyA-4)LETTFWGDGE (SEQ ID NO: 11).

49. The polypeptide according to any one of the preceding claims, wherein the polypeptide is a cyclic polypeptide comprising or consisting of the amino acid sequence TX₁LETTFX₄GDGEPKTIRVTQpGX₁₀ (SEQ ID NO: 13)
wherein
X₁ is H or H-3Me;
X₄ is W or absent;
X₁₀ is C, Q or E;
or a pharmaceutically acceptable salt thereof.

50. The polypeptide according to any one of the preceding claims, wherein the polypeptide is a cyclic polypeptide comprising or consisting of the amino acid sequence THLETTFWGDGEPKTIRVTQ (SEQ ID NO: 419).

51. The polypeptide according to any one of the preceding claims, wherein the polypeptide is a cyclic polypeptide comprising or consisting of the amino acid sequence THLETTFGDGEPKTIRVTQ (SEQ ID NO: 420).

52. The polypeptide according to any one of the preceding claims, wherein the polypeptide is a cyclic polypeptide comprising or consisting of the amino acid sequence TH(3-Me)LETTFWGDGEPKTIRVTQ (SEQ ID NO: 421).

53. The polypeptide according to any one of the preceding claims, wherein the polypeptide is a cyclic polypeptide comprising or consisting of the amino acid sequence TH(3-Me)LETTFGDGEPKTIRVTQ (SEQ ID NO: 422).

54. The polypeptide according to any of the preceding claims, wherein the polypeptide comprises at least 20 amino acid residues, such as at least 21 amino acid residues, such as at least 22 amino acid residues, such as at least 23 amino acid residues, such as at least 24 amino acid residues, such as at least 25 amino acid residues, such as at least 26 amino acid residues, such as at least 27 amino acid residues, such as at least 28 amino acid residues, such as at least 29 amino acid residues, such as at least 30 amino acid residues, such as at least 31 amino acid residues, such as at least 32 amino acid residues, such as at least 33 amino acid residues, such as at least 34 amino acid residues, such as at least 35 amino acid residues, such as at least 36 amino acid residues, such as at least 37 amino acid residues.

55. The polypeptide according to any of the preceding claims, wherein the polypeptide comprises no more than 50 amino acid residues, such as no more than 45 amino acid residues, such as no more than 40 amino acid residues, such as no more than 35 amino acid residues, such as no more than 30 amino acid residues, such as no more than 29 amino acid residues, such as no more than 28 amino acid residues, such as no more than 27 amino acid residues, such as no more than 26 amino acid residues, such as no more than 25 amino acid residues, such as no more than 24 amino acid residues, such as no more than 23 amino acid residues, such as no more than 22 amino acid residues, such as no more than 21 amino acid residues, such as no more than 20 amino acid residues.

56. The polypeptide according to any of the preceding claims, wherein the polypeptide comprises in the range of 19 to 50 amino acid residues, such as in the range of 19 to 45 amino acid residues, such as in the range of 19 to 40 amino acid residues, such as in the range of 19 to 35 amino acid residues, such as in the range of 19 to 30 amino acid residues, such as in the range of 19 to 25 amino acid residues, such as in the range of 19 to 23 amino acid residues, such as in the range of 20 to 23 amino acid residues, such as in the range of 20 to 22 amino acid residues.

57. The polypeptide according to any one of the preceding claims, wherein the polypeptide is capable of binding to PSD-95.

58. The polypeptide according to any one of the preceding claims, wherein the polypeptide is capable of inhibiting binding of nNOS to the PDZ2 domain of PSD-95.

59. The polypeptide according to any one of the preceding claims, wherein the polypeptide binds to PSD-95-PDZ2 with a K_{d} of less than 100 μM, such as less than 75 μM, such as less than 50 μM, such as less than 25 μM, such as less than 20 μM, such as less than 15 μM, such as less than 10 μM, such as less than 5 μM, such as less than 4 μM, such as less than 3 μM, such as less than 2 μM, such as less than 1 μM.

60. The polypeptide according to any one of the preceding claims, wherein the compound has a K value for inhibiting binding of nNOS to PDZ2 domain of PSD-95 of less than 100 μM, such as less than 75 μM, such as less than 50 μM, such as less than 10 μM, such as less than 5 μM, such as less than 2.5 μM, such as less than 1 μM.

61. The polypeptide according to any one of the preceding claims, wherein the polypeptide is further conjugated to a moiety.

62. The polypeptide according to claim 61, wherein the moiety is selected from the group consisting of PEG, monosaccharides, fluorophores, chromophores, radioactive compounds, and cell-penetrating peptides.

63. The polypeptide according to claim 61, wherein the moiety is a detectable moiety.

64. The polypeptide according to any one of the preceding claims, wherein the polypeptide is further modified by glycosylation, PEGylation, amidation, esterification, acylation, acetylation and/or alkylation.

65. The polypeptide according to any one of the preceding claims, wherein one or more of the amino acid residues are alkylated, such as methylated.

66. The polypeptide according to any one of claims 1, 10, 11, or 54 to 65, wherein the polypeptide comprises or consist of an amino acid sequence selected from the group consisting of SEQ ID NO: 14 to 136 and SEQ ID NO: 139 to 433.

67. The polypeptide according to any of the preceding claims, wherein the polypeptide is cyclo-(THLETTFWGDGEPKTIRVTQpG(Eα)) (SEQ ID NO: 423).

68. The polypeptide according to any of the preceding claims, wherein the polypeptide is cyclo-(THLETTFGDGEPKTIRVTQpG(Eα)) (SEQ ID NO: 424).

69. The polypeptide according to any of the preceding claims, wherein the polypeptide is cyclo-(TH(3-Me)LETTFWGDGEPKTIRVTQpG(Eα)) (SEQ ID NO: 425).

70. The polypeptide according to any of the preceding claims, wherein the polypeptide is cyclo-(TH(3-Me)LETTFGDGEPKTIRVTQpG(Eα)) (SEQ ID NO: 426).

71. The polypeptide according to any of the preceding claims, wherein the polypeptide is cyclo-(THLETTFWGDGEPKTIRVTQpG(Qα)) (SEQ ID NO: 14).

72. The polypeptide according to any of the preceding claims, wherein the polypeptide is cyclo-(THLETTFGDGEPKTIRVTQpG(Qα)) (SEQ ID NO: 15).

73. The polypeptide according to any of the preceding claims, wherein the polypeptide is cyclo-(TH(3-Me)LETTFWGDGEPKTIRVTQpG(Qα)) (SEQ ID NO: 16).

74. The polypeptide according to any of the preceding claims, wherein the polypeptide is cyclo-(TH(3-Me)LETTFGDGEPKTIRVTQpG(Qα)) (SEQ ID NO: 17).

75. A composition comprising the polypeptide according to any of the preceding claims.

76. The composition according to claim 75, wherein the composition is a pharmaceutical composition.

77. A polynucleotide encoding the polypeptide as defined in any one of claims 1 to 74.

78. A vector comprising a polynucleotide as defined in claim 77.

79. A host cell comprising the polynucleotide according to claim 77 or the vector according to claim 78.

80. The host cell according to claim 79, wherein the host cell is a bacterial cell.

81. The host cell according to claim 79, wherein the host cell is a mammalian cell.

82. The host cell according to claim 79, wherein the host cell is a human cell.

83. The polypeptide according to any one of claims 1 to 74, the composition according to claim 75, the polynucleotide according to claim 77, the vector according to claim 78, or the host cell according to claim 79 for use as a medicament.

84. The polypeptide according to any one of claims 1 to 74, the composition according to claim 75, the polynucleotide according to claim 77, the vector according to claim 78, or the host cell according to claim 79 for use in prevention and/or treatment of an excitotoxic-related disease in a subject.

85. The polypeptide according to any one of claims 1 to 74, the composition according to claim 75, the polynucleotide according to claim 77, the vector according to claim 78, or the host cell according to claim 79 for use according to claim 84, wherein the excitotoxic-related disease is stroke, such as ischemic stroke.

86. The polypeptide according to any one of claims 1 to 74, the composition according to claim 75, the polynucleotide according to claim 77, the vector according to claim 78, or the host cell according to claim 79 for use according to claim 85, wherein the polypetide is administered in combination with reperfusion therapy, such as in combination with administration of a thrombolytic agent.

87. The polypeptide according to any one of claims 1 to 74, the composition according to claim 75, the polynucleotide according to claim 77, the vector according to claim 78, or the host cell according to claim 79 for use according to claim 84, wherein the excitotoxic-related disease is ischemic or traumatic injury of the CNS, such as spinal cord injury and traumatic brain injury.

88. The polypeptide according to any one of claims 1 to 74, the composition according to claim 75, the polynucleotide according to claim 77, the vector according to claim 78, or the host cell according to claim 79 for use according to claim 84, wherein the excitotoxic-related disease is epilepsy.

89. The polypeptide according to any one of claims 1 to 74, the composition according to claim 75, the polynucleotide according to claim 77, the vector according to claim 78, or the host cell according to claim 79 for use according to claim 84, wherein the excitotoxic-related disease is a neurodegenerative disease of the CNS.

90. The polypeptide according to any one of claims 1 to 74, the composition according to claim 75, the polynucleotide according to claim 77, the vector according to claim 78, or the host cell according to claim 79 for use according to claim 84, wherein the neurodegenerative disease of the CNS is selected from the group consisting of Alzheimer's disease, Huntington's disease and Parkinson's disease.

91. The polypeptide according to any one of claims 1 to 74, the composition according to claim 75, the polynucleotide according to claim 77, the vector according to claim 78, or the host cell according to claim 79 for use in prevention and/or treatment of neuropathic pain in a subject.

92. A method of preventing and/or treating an excitotoxicity-related disease and/or neuropathic pain, said method comprising administering a therapeutically effective amount of the polypeptide according to any one of claims 1 to 74, the composition according to claim 75, the polynucleotide according to claim 77, the vector according to claim 78, or the host cell according to claim 79, to a subject in need thereof.

93. The method according to claim 92, wherein the polypeptide according to any one of claims 1 to 74, the composition according to claim 75, the polynucleotide according to claim 77, the vector according to claim 78, or the host cell according to claim 79, is administered in combination with reperfusion therapy, such as in combination with administration of a thrombolytic agent.

94. Use of the polypeptide according to any one of claims 1 to 74, the composition according to claim 75, the polynucleotide according to claim 77, the vector according to claim 78, or the host cell according to claim 79, for the manufacture of a medicament for the treatment and/or prevention of an excitotoxicity-related disease and/or neuropathic pain in a subject.

95. A kit of parts comprising at least two separate unit dosage forms (A) and (B), wherein
(A) comprises the polypeptide according to any one of claims 1 to 74, the composition according to claim 75, the polynucleotide according to claim 77, the vector according to claim 78, or the host cell according to claim 79; and
(B) comprises a thrombolytic agent.

96. The kit of parts according to claim 95 for use in in treating, preventing, reducing and/or delaying development of an excitotoxic-related disease and/or pain, wherein (A) and (B) are administered simultaneously, sequentially or separately to the subject.

97. A method for manufacturing the polypeptide according to any of claims 1 to 74, said method comprising the steps of:
a) preparing a peptide using Fmoc/tBu-based solid-phase peptide synthesis (SPPS), and
b) cyclization of said peptide via native chemical ligation (NCL).

98. The method according to claim 97, wherein step b) involves oxidizing a C-terminal hydrazine group to an azide and reacting said azide with a thiol group of the N-terminal Cys, followed by transthioesterification to form an amide bond linkage.

99. The method according to claim 97, further comprising a step following step b), wherein a fluorophore is conjugated to the polypeptide.

100. A method for manufacturing the polypeptide according to any of claims 1 to 74, said method comprising the steps of:
a) Providing a cellulose membrane;
b) Coupling of PEG spacer and adding a mixture of Fmoc/Boc-Gly to the cellulose membrane provided in step a);
c) Capping the membrane prepared in step b) with acetic anhydride;
d) Adding quasi-orthogonal protected AA to the product of step c);
e) Preparing the remaining polypeptide using Fmoc/tBu-based solid-phase peptide synthesis (SPPS) on the AA of step d);
f) Removing the quasi-orthogonal protecting group from the polypeptide generated in step e) and cyclizing the polypeptide;
g) Cleaving side-chain protecting groups from the polypeptide generated in step f); and
h) Cleaving the polypeptide from the cellulose membrane.
